(19) United States
(12) Patent Application Publication
(10) Pub. No.: US 2007/0105900 A1
(43) Pub. Date: May 10, 2007

(54) Pharmaceutical compounds

(75) Inventors: Valerio Berdini, Cambridge (GB); Andrew James Woodhead, Cambridge (GB); Paul Graham Wyatt, Perth (GB); Michael Alistair O'Brien, Cambridge (GB); Eva Figueroa Navarro, Cambridge (GB)

Correspondence Address: HESLIN ROTHENBERG FARLEY & MESITI PC, 5 COLUMBIA CIRCLE, ALBANY, NY, 12203, US

(73) Assignee: Atex Therapeutics, Limited, Cambridge (GB)

(21) Appl. No.: 10/563,350

(22) PCT Filed: Jul. 4, 2004
(86) PCT No.: PCT/GB04/02913

§ 371 Date: Aug. 21, 2006

Related U.S. Application Data
(60) Provisional application No. 60/484,685, filed on Jul. 3, 2003.
(60) Provisional application No. 60/514,170, filed on Oct. 24, 2003.

(30) Foreign Application Priority Data
Jul. 3, 2003 (GB) 0315657.7

(51) Int. Cl. A61K 31/4709; A61K 31/4745; A61K 31/422; A61K 31/42; A61K 31/4439; A61K 31/4178; A61K 31/416; C07D 403/14; C07D 471/02

(52) U.S. Cl. 514/314; 514/397; 514/406; 548/311.1; 548/364.1; 514/341; 514/378; 514/374; 546/176; 548/215; 546/272.7; 514/300; 546/113; 514/303; 546/119

(57) ABSTRACT

The invention provides compounds of the formula (I): 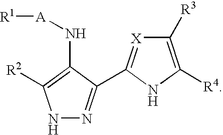 The compounds have activity against cyclin dependent kinases, glycogen synthase kinase and Auroa kinases and are therefore useful to treat cancer and viral diseases.

This invention relates to pyrazole compounds that inhibit or modulate the activity of Cyclin Dependent Kinases (CDK), Glycogen Synthase Kinases (GSK) and Aurora kinases to the use of the compounds in the treatment or prophylaxis of disease states or conditions mediated by the kinases, and to novel compounds having kinase inhibitory or modulating activity. Also provided are pharmaceutical compositions containing the compounds and novel chemical intermediates.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J., 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, disease and conditions of the immune system, disease and conditions of the central nervous system, and angiogenesis.

The process of eukaryotic cell division may be broadly divided into a series of sequential phases termed G1, S, G2 and M. Correct progression through the various phases of the cell cycle has been shown to be critically dependent upon the spatial and temporal regulation of a family of proteins known as cyclin dependent kinases (cdks) and a diverse set of their cognate protein partners termed cyclins. Cdks are cdc2 (also known as cdk1) homologous serine-threonine kinase proteins that are able to utilise ATP as a substrate in the phosphorylation of diverse polypeptides in a sequence dependent context. Cyclins are a family of proteins characterised by a homology region, containing approximately 100 amino acids, termed the “cyclin box” which is used in binding to, and defining selectivity for, specific cdk partner proteins.

Modulation of the expression levels, degradation rates, and activation levels of various cdks and cyclins throughout the cell cycle leads to the cyclical formation of a series of cdk/cyclin complexes, in which the cdks are enzymatically active. The formation of these complexes controls passage through discrete cell cycle checkpoints and thereby enables the process of cell division to continue. Failure to satisfy the pre-requisite biochemical criteria at a given cell cycle checkpoint, i.e. failure to form a required cdk/cyclin complex, can lead to cell cycle arrest and/or cellular apoptosis. Aberrant cellular proliferation, as manifested in cancer, can often be attributed to loss of correct cell cycle control. Inhibition of cdk enzymatic activity therefore provides a means by which abnormally dividing cells can have their division arrested and/or be killed. The diversity of cdks, and cdk complexes, and their critical roles in mediating the cell cycle, provides a broad spectrum of potential therapeutic targets selected on the basis of a defined biochemical rationale. Progression from the G1 phase to the S phase of the cell cycle is primarily regulated by cdk2, cdk3, cdk4 and cdk6 via association with members of the D and E type cyclins. The D-type cyclins appear instrumental in enabling passage beyond the G1 restriction point, where as the cdk2/cyclin E complex is key to the transition from the G1 to S phase. Subsequent progression through S phase and entry into G2 is thought to require the cdk2/cyclin A complex. Both mitosis, and the G2 to M phase transition which triggers it, are regulated by complexes of cdk1 and the A and B type cyclins.

During G1 phase Retinoblastoma protein (Rb), and related pocket proteins such as p130, are substrates for cdk(2, 4, & 6)/cyclin complexes. Progression through G1 is in part facilitated by hyperphosphorylation, and thus inactivation, of Rb and p130 by the cdk(4/6)/cyclin-D complexes. Hyperphosphorylation of Rb and p130causes the release of transcription factors, such as E2F, and thus the expression of genes necessary for progression through G1 and for entry into S-phase, such as the gene for cyclin E. Expression of cyclin E facilitates formation of the cdk2/cyclin E complex which amplifies, or maintains, E2F levels via further phosphorylation of Rb. The cdk2/cyclin E complex also phosphorylates other proteins necessary for DNA replication, such as NPAT, which has been implicated in histone biosynthesis. G1 progression and the G1/S transition are also regulated via the mitogen stimulated Myc pathway, which feeds into the cdk2/cyclin E pathway. Cdk2 is also connected to the p53 mediated DNA damage response pathway via p53 regulation of p2l levels. p21 is a protein inhibitor of cdk2/cyclin E and is thus capable of blocking, or delaying, the G1/S transition. The cdk2/cyclin E complex may thus represent a point at which biochemical stimuli from the Rb, Myc and p53 pathways are to some degree integrated. Cdk2 and/or the cdk2/cyclin E complex therefore represent good targets for therapeutics designed at arresting, or recovering control of, the cell cycle in aberrantly dividing cells.

The exact role of cdk3 in the cell cycle is not clear. As yet no cognate cyclin partner has been identified, but a dominant negative form of cdk3 delayed cells in G1, thereby suggesting that cdk3 has a role in regulating the G1/S transition.

Although most cdks have been implicated in regulation of the cell cycle there is evidence that certain members of the cdk family are involved in other biochemical processes. This is exemplified by cdk5 which is necessary for correct neuronal development and which has also been implicated in the phosphorylation of several neuronal proteins such as Tau, NUDE-1, synapsin1, DARPP32 and the Munc18/Syntaxin1A complex. Neuronal cdk5 is conventionally activated by binding to the p35/p39 proteins. Cdk5 activity can, however, be deregulated by the binding of p25, a truncated version of p35. Conversion of p35 to p25, and subsequent deregulation of cdk5 activity, can be induced by ischemia, excitotoxicity, and β-amyloid peptide. Consequently p25 has been implicated in the pathogenesis of neurodegenerative diseases, such as Alzheimer's, and is therefore of interest as a target for therapeutics directed against these diseases.

Cdk7 is a nuclear protein that has cdc2 CAK activity and binds to cyclin H. Cdk7 has been identified as component of the TFIIH transcriptional complex which has RNA polymerase II C-terminal domain (CTD) activity. This has been associated with the regulation of HIV-1 transcription via a Tat-mediated biochemical pathway. Cdk8 binds cyclin C and has been implicated in the phosphorylation of the CTD of RNA polymerase II. Similarly the cdk9/cyclin-T1 complex (P-TEFb complex) has been implicated in elongation control of RNA polymerase II. PTEF-b is also required for activation of transcription of the HIV-1 genome by the viral transactivator Tat through its interaction with cyclin T1. Cdk7, cdk8, cdk9 and the P-TEFb complex are therefore potential targets for anti-viral therapeutics. At a molecular level mediation of cdk/cyclin complex activity requires a series of stimulatory and inhibitory phosphorylation, or dephosphorylation, events. Cdk phosphorylation is performed by a group of cdk activating kinases (CAKs) and/or kinases such as weel, Myt1 and Mik1. Dephosphorylation is performed by phosphatases such as cdc25(a & c), pp2a, or KAP.

Cdk/cyclin complex activity may be further regulated by two families of endogenous cellular proteinaceous inhibitors: the Kip/Cip family, or the INK family. The INK proteins specifically bind cdk4 and cdk6. p16^{ink4} (also known as MTS1) is a potential tumour suppressor gene that is mutated, or deleted, in a large number of primary cancers. The Kip/Cip family contains proteins such as p21^{Cip1,Waf1}, p27^{Kip1} and p57^{kip2}. As discussed previously p21 is induced by p53 and is able to inactivate the cdk2/cyclin(E/A) and cdk4/cyclin(D1/D2/D3) complexes. Atypically low levels of p27 expression have been observed in breast, colon and prostate cancers. Conversely over expression of cyclin E in solid tumours has been shown to correlate with poor patient prognosis. Over expression of cyclin D1 has been associated with oesophageal, breast, squamous, and non-small cell lung carcinomas.

The pivotal roles of cdks, and their associated proteins, in co-ordinating and driving the cell cycle in proliferating cells have been outlined above. Some of the biochemical pathways in which cdks play a key role have also been described. The development of monotherapies for the treatment of proliferative disorders, such as cancers, using therapeutics targeted generically at cdks, or at specific cdks, is therefore potentially highly desirable. Cdk inhibitors could conceivably also be used to treat other conditions such as viral infections, autoimrnmune diseases and neuro-degenerative diseases, amongst others. Cdk targeted therapeutics may also provide clinical benefits in the treatment of the previously described diseases when used in combination therapy with either existing, or new, therapeutic agents. Cdk targeted anticancer therapies could potentially have advantages over many current antitumour agents as they would not directly interact with DNA and should therefore reduce the risk of secondary tumour development.

Aurora Kinases

Relatively recently, a new family of serine/threonine kinases known as the Aurora kinases has been discovered that are involved in the G2 and M phases of the cell cycle, and which are important regulators of mitosis.

The precise role of Aurora kinases has yet to be elucidated but that they play a part in mitotic checkpoint control, chromosome dynamics and cytokinesis (Adams et al., Trends Cell Biol., 11: 49-54 (2001). Aurora kinases are located at the centrosomes of interphase cells, at the poles of the bipolar spindle and in the mid-body of the mitotic apparatus.

Three members of the Aurora kinase family have been found in mammals so far (E. A. Nigg, Nat. Rev. Mol. Cell Biol. 2: 21-32, (2001)). These are:
Aurora A (also referred to in the literature as Aurora 2);
Aurora B (also referred to in the literature as Aurora 1); and
Aurora C (also referred to in the literature as Aurora 3).

The Aurora kinases have highly homologous catalytic domains but differ considerably in their N-terminal portions (Katayama H, Brinkley W R, Sen S.; The Aurora kinases: role in cell transformation and tumorigenesis; Cancer Metastasis Rev. 2003 December; 22(4):451-64).

The substrates of the Aurora kinases A and B have been identified as including a kinesin-like motor protein, spindle apparatus proteins, histone H3 protein, kinetochore protein and the tumour suppressor protein p53.

Aurora A kinases are believed to be involved in spindle formation and become localised on the centrosome during the early G2 phase where they phosphorylate spindle-associated proteins (Prigent et al., Cell, 114: 531-535 (2003). Hirota et al, Cell, 114:585-598, (2003) found that cells depleted of Aurora A protein kinase were unable to enter mitosis. Furthermore, it has been found (Adams, 2001) that mutation or disruption of the Aurora A gene in various species leads to mitotic abnormalities, including centrosome separation and maturation defects, spindle aberrations and chromosome segregation defects.

The Aurora kinases are generally expressed at a low level in the majority of normal tissues, the exceptions being tissues with a high proportion of dividing cells such as the thymus and testis. However, elevated levels of Aurora kinases have been found in many human cancers (Giet et al., J. Cell. Sci. 112: 3591-361, (1999) and Katayama (2003). Furthermore, Aurora A kinase maps to the chromosome 20q13 region that has frequently been found to be amplified in many human cancers.

Thus, for example, significant Aurora A over-expression has been detected in human breast, ovarian and pancreatic cancers (see Zhou et al., Nat. Genet. 20: 189-193, (1998), Tanaka et al., Cancer Res., 59: 2041-2044, (1999) and Han et al., cancer Res., 62: 2890-2896, (2002).

Moreover, Isola, American Journal of Pathology 147,905-911 (1995) has reported that amplification of the Aurora A locus (20q13) correlates with poor prognosis for patients with node-negative breast cancer.

Amplification and/or over-expression of Aurora-A is observed in human bladder cancers and amplification of Aurora-A is associated with aneuploidy and aggressive clinical behaviour, see Sen et al., J. Natl. Cancer Inst, 94: 1320-1329 (2002).

Elevated expression of Aurora-A has been detected in over 50% of colorectal cancers, (see Bischoff et al., EMBO J., 17: 3052-3065, (1998) and Takahashi et al., Jpn. J. Cancer Res. , 91: 1007-1014 (2000)) ovarian cancers (see Gritsko et al. Clin. Cancer Res., 9: 1420-1426 (2003), and gastric tumours Sakakura et al., British Journal of Cancer, 84: 824-831 (2001).

Tanaka et al. Cancer Research, 59: 2041-2044 (1999) found evidence of over-expression of Aurora A in 94% of invasive duct adenocarcinomas of the breast.

High levels of Aurora A kinase have also been found in renal, cervical, neuroblastoma, melanoma, lymphoma, pancreatic and prostate tumour cell lines Bischoff et al. (1998), EMBO J., 17: 3052-3065 (1998); Kimura et al. J. Biol. Chem., 274: 7334-7340 (1999); Zhou et al., Nature Genetics, 20: 189-193 (1998); Li et al., Clin Cancer Res. 9 (3): 991-7 (2003)].

Aurora-B is highly expressed in multiple human tumour cell lines, including leukemic cells [Katayama et al., Gene 244: 1-7)]. Levels of this enzyme increase as a function of Duke's stage in primary colorec-tal cancers [Katayama et al., J. Natl Cancer Inst., 91: 1160-1162 (1999)].

High levels of Aurora-3 (Aurora-C) have been detected in several tumour cell lines, even though this kinase tends to be restricted to germ cells in normal tissues (see Kimura et al. Journal of Biological Chemistry, 274: 7334-7340 (1999)). Over-expression of Aurora-3 in approximately 50% of colorectal cancers has also been reported in the article by Takahashi et al., Jpn J. Cancer Res. 91: 1007-1014 (2001)].

Other reports of the role of Aurora kinases in proliferative disorders may be found in Bischoff et al., Trends in Cell Biology 9: 454-459 (1999); Giet et al. Journal of Cell Science, 112: 3591-3601 (1999) and Dutertre, et al. Oncogene, 21: 6175-6183 (2002).

Royce et al report that the expression of the Aurora 2 gene (known as STK15 or BTAK) has been noted in approximately one-fourth of primary breast tumours. (Royce M E, xia W, Sahin A A, Katayama H, Johnston D A, Hortobagyi G, Sen S, Hung M C; STK15/Aurora-A expression in primary breast tumours is correlated with nuclear grade but not with prognosis; Cancer. 2004 Jan. 1;100(1):12-9).

Endometrial carcinoma (EC) comprises at least two types of cancer: endometrioid carcinomas (EECs) are estrogen-related tumours, which are frequently euploid and have a good prognosis. Nonendometrioid carcinomas (NEECs; serous and clear cell forms) are not estrogen related, are frequently aneuploid, and are clinically aggressive. It has also been found that Aurora was amplified in 55.5% of NEECs but not in any EECs (P< or =0.001) (Moreno-Bueno G, Sanchez-Estevez C, Cassia R, Rodriguez-Perales S, Diaz-Uriarte R, Dominguez O, Hardisson D, Andujar M, Prat J, Matias-Guiu X, Cigudosa J C, Palacios J. Cancer Res. 2003 Sep. 15;63(18):5697-702).

Reichardt et al (Oncol Rep. 2003 September-October;10(5):1275-9)have reported that quantitative DNA analysis by PCR to search for Aurora amplification in gliomas revealed that five out of 16 tumours (31%/u) of dilfferent WHO grade (1× grade II, 1× grade III, 3× grade IV) showed DNA amplification of the Aurora 2 gene. It was hypothesized that amplification of the Aurora 2 gene may be a non-random genetic alteration in human gliomas playing a role in the genetic pathways of tumourigenesis.

Results by Hamada et al (Br. J. Haematol. 2003 May;121(3):439-47) also suggest that Aurora 2 is an effective candidate to indicate not only disease activity but also tumourigenesis of non-Hodgkin's lymphoma. Retardation of tumour cell growth resulting from the restriction of this gene's functions could be a therapeutic approach for non-Hodgkin's lymphoma.

In a study by Gritsko et al (Clin Cancer Res. 2003 April; 9(4):1420-6)), the kinase activity and protein levels of Aurora A were examined in 92 patients with primary ovarian tumours. In vitro kinase analyses revealed elevated Aurora A kinase activity in 44 cases (48%). Increased Aurora A protein levels were detected in 52 (57%) specimens. High protein levels of Aurora A correlated well with elevated kinase activity.

Results obtained by Li et al (Clin. Cancer Res. 2003 March; 9(3):991-7) showed that the Aurora A gene is overexpressed in pancreatic tumours and carcinoma cell lines and suggest that overexpression of Aurora A may play a role in pancreatic carcinogenesis.

Similarly, it has been shown that Aurora A gene amplification and associated increased expression of the mitotic kinase it encodes are associated with aneuploidy and aggressive clinical behaviour in human bladder cancer. (J. Natl. Cancer Inst. 2002 Sep. 4; 94(17):1320-9).

Investigation by several groups (Dutertre S, Prigent C., Aurora-A overexpression leads to override of the microtubule-kinetochore attachment checkpoint; Mol. Interv. 2003 May; 3(3):127-30 and Anand S, Penrhyn-Lowe S, Venkitaraman A R., Aurora-A amplification overrides the mitotic spindle assembly checkpoint, inducing resistance to Taxol, Cancer Cell. 2003 Janurary;3(1):5 1-62) suggests that overexpression of Aurora kinase activity is associated with resistance to some current cancer therapies. For example overexpression of Aurora A in mouse embryo fibroblasts can reduce the sensitivity of these cells to the cytotoxic effects of taxane derivatives. Therefore Aurora kinase inhibitors may find particular use in patients who have developed reistance to existing therapies.

On the basis of work carried out to date, it is envisaged that inhibition of Aurora kinases, particularly Aurora kinase A and Aurora kinase B, will prove an effective means of arresting tumour development.

Harrington et al (Nat Med. 2004 March;10(3):262-7) have demonstrated that an inhibitor of the Aurora kinases suppresses tumour growth and induces tumour regression in vivo. In the study, the Aurora kinase inhibitor blocked cancer cell proliferation, and also triggered cell death in a range of cancer cell lines including leukaemic, colorectal and breast cell lines.

Cancers which may be particularly amenable to Aurora inhibitors include breast, bladder, colorectal, pancreatic, ovarian, non-Hodgkin's lymphoma, gliomas and nonendometrioid endometrial carcinomas.

Glycogen Synthase Kinase

Glycogen Synthase Kinase-3 (GSK3) is a serine-threonine kinase that occurs as two ubiquitously expressed isoforms in humans (GSK3α & beta GSK3β). GSK3 has been implicated as having roles in embryonic development, protein synthesis, cell proliferation, cell differentiation, microtubule dynamics, cell motility and cellular apoptosis. As such GSK3 has been implicated in the progression of disease states such as diabetes, cancer, Alzheimer's disease, stroke, epilepsy, motor neuron disease and/or head trauma. Phylogenetically GSK3 is most closely related to the cyclin dependent kinases (CDKs).

The consensus peptide substrate sequence recognised by GSK3 is (Ser/Thr)-X-X-X-(pSer/pThr), where X is any amino acid (at positions (n+1), (n+2), (n+3)) and pSer and pTbr are phospho-serine and phospho-th-eonine respectively (n+4). GSK3 phosphorylates the first serine, or threonine, at position (n). Phospho-serine, or phospho-threonine, at the (n+4) position appear necessary for priming GSK3 to give maximal substrate turnover. Phosphorylation of GSK3α at Ser21, or GSK3β at Ser9, leads to inhibition of GSK3. Mutagenesis and peptide competition studies have led to the model that the phosphorylated N-terminus of GSK3 is able to compete with phospho-peptide substrate (S/TXXXpS/pT) via an autoinhibitory mechanism. There are also data suggesting that GSK3α and GSKβ may be subtly regulated by phosphorylation of tyrosines 279 and 216 respectively. Mutation of these residues to a Phe caused a reduction in in vivo kinase activity. The X-ray crystallographic structure of GSK3β has helped to shed light on all aspects of GSK3 activation and regulation.

GSK3 forms part of the mamnmalian insulin response pathway and is able to phosphorylate, and thereby inactivate, glycogen synthase. Upregulation of glycogen synthase activity, and thereby glycogen synthesis, through inhibition of GSK3, has thus been considered a potential means of combating type II, or non-insulin-dependent diabetes mellitus NIDDM): a condition in which body tissues become resistant to insulin stimulation. The cellular insulin response in liver, adipose, or muscle tissues, is triggered by insulin binding to an extracellular insulin receptor. This causes the phosphorylation, and subsequent recruitment to the plasma membrane, of the insulin receptor substrate (IRS) proteins. Further phosphorylation of the IRS proteins initiates recruitment of phosphoinositide-3 kinase (PI3K) to the plasma membrane where it is able to liberate the second messenger phosphatidylinosityl 3,4,5-trisphosphate (PIP3). This facilitates co-localisation of 3-phosphoinositide-dedependent protein kinase 1 (PDK1) and protein kinase B (PKB or Akt) to the membrane, where PDK1 activates PKB. PKB is able to phosphorylate, and thereby inhibit, GSK3α and/or GSKβ through phosphorylation of Ser9, or ser21, respectively. The inhibition of GSK3 then triggers upregulation of glycogen synthase activity. Therapeutic agents able to inhibit GSK3 may thus be able to induce cellular responses akin to those seen on insulin stimulation. A fuiher in vivo substrate of GSK3 is the eukaryotic protein synthesis initiation factor 2B (eIF2B). eIF2B is inactivated via phosphorylation and is thus able to suppress protein biosynthesis. Inhibition of GSK3, e.g. by inactivation of the “mammalian target of rapamycin” protein (mTOR), can thus upregulate protein biosynthesis. Finally there is some evidence for regulation of GSK3 activity via the mitogen activated protein kinase (MAPK) pathway through phosphorylation of GSK3 by kinases such as mitogen activated protein kinase activated protein kinase 1 (MAPKAP-K1 or RSK). These data suggest that GSK3 activity may be modulated by mitogenic, insulin and/or amino acid stimulii.

It has also been shown that GSK3β is a key component in the vertebrate Wnt signalling pathway. This biochemical pathway has been shown to be critical for normal embryonic development and regulates cell proliferation in normal tissues. GSK3 becomes inhibited in response to Wnt stimulii. This can lead to the de-phosphorylation of GSK3 substrates such as Axin, the adenomatous polyposis coli (APC) gene product and β-catenin. Aberrant regulation of the Wnt pathway has been associated with many cancers. Mutations in APC, and/or β-catenin, are common in colorectal cancer and other tumours. β-catenin has also been shown to be of importance in cell adhesion. Thus GSK3 may also modulate cellular adhesion processes to some degree. Apart from the biochemical pathways already described there are also data implicating GSK3 in the regulation of cell division via phosphorylation of cyclin-D1, in the phosphorylation of transcription factors such as c-Jun, CCAAT/enhancer binding protein a (C/EBPa), c-Myc and/or other substrates such as Nuclear Factor of Activated T-cells (NFATc), Heat Shock Factor-1 (HSF-1) and the c-AMP response element binding protein (CREB). GSK3 also appears to play a role, albeit tissue specific, in regulating cellular apoptosis. The role of GSK3 in modulating cellular apoptosis, via a pro-apoptotic mechanism, may be of particular relevance to medical conditions in which neuronal apoptosis can occur. Examples of these are head trauma, stroke, epilepsy, Alzheimer's and motor neuron diseases, progressive supranuclear palsy, corticobasal degeneration, and Pick's disease. In vitro it has been shown that GSK3 is able to hyper-phosphorylate the microtub-ule associated protein Tau. Hyperphosphorylation of Tau disrupts its normal binding to microtubules and may also lead to the formation of intra-cellular Tau filaments. It is believed that the progressive accumulation of these filaments leads to eventual neuronal dysfunction and degeneration. Inhbition of Tau phosphorylation, through inhibition of GSK3, may thus provide a means of limiting and/or preventing neurodegenerative effects.

WO 02/34721 from Du Pont discloses a class of indeno [1,2-c]pyrazol-4-ones as inhibitors of cyclin dependent kinases.

WO 01/81348 from Bristol Myers Squibb describes the use of 5-thio-, sulfinyl- and sulfonylpyrazolo[3,4-b]-pyridines as cyclin dependent kinase inhibitors.

WO 00/62778 also from Bristol Myers Squibb discloses a class of protein tyrosine kinase inhibitors.

WO 01/72745A1 from Cyclacel describes 2-substituted 4-heteroaryl-pyrimidines and their preparation, pharmaceutical compositions containing them and their use as inhibitors of cyclin-dependant kinases (cdks) and hence their use in the treatment of proliferative disorders such as cancer, leukaemia, psoriasis and the like.

WO 99/21845 from Agouron describes 4-aminothiazole derivatives for inhibiting cyclin-dependent kinases (cdks), such as CDK1, CDK2, CDK4, and CDK6. The invention is also directed to the therapeutic or prophylactic use of pharmaceutical compositions containing such compounds and to methods of treating malignancies and other disorders by administering effective amounts of such compounds.

WO 01/53274 from Agouron discloses as CDK kinase inhibitors a class of compounds which can comprise an amide-substituted benzene ring linked to an N-containing heterocyclic group. Although indazole compounds are not mentioned generically, one of the exemplified compounds comprises an indazole 3-carboxylic acid anilide moiety linked via a methylsulfanyl group to a pyrazolopyrimidine.

WO 01/98290 (Pharmacia & Upjohn) discloses a class of 3-aminocarbonyl-2-carboxamido thiophene derivatives as protein kinase inhibitors. The compounds are stated to nave multiple protein kinase activity.

WO 01/53268 and WO 01/02369 from Agouron disclose compounds that mediate or inhibit cell proliferation through the inhibition of protein kinases such as cyclin dependent kinase or tyrosine kinase. The Agouron compounds have an aryl or heteroaryl ring attached directly or though a CH═CH or CH═N group to the 3-position of an indazole ring.

WO 00/39108 and WO 02/00651 (both to Du Pont Pharmaceuticals) describe broad classes of heterocyclic compounds that are inhibitors of trypsin-like serine protease enzymes, especially factor Xa and thrombin. The compounds are stated to be useful as anticoagulants or for the prevention of thromboembolic disorders.

Heterocyclic compounds that have activity against factor Xa are also disclosed in WO 01/1978 Cor Therapeutics) and US 2002/0091116 (Zhu et al.).

WO 03/035065 (Aventis) discloses a broad class of benzimidazole derivatives as protein kinase inhibitors but does not disclose activity against CDK kinases or GSK kinases.

WO 97/36585 and U.S. Pat. No. 5,874,452 (both to Merck) disclose biheteroaryl compounds that are inhibitors of farnesyl transferase.

WO 03/037274 (Icagen) discloses pyrazole amides as inhibitors of sodium channels.

WO 00/43384 (Boehringer Ingelheim) discloses aryl and heteroaryl ureas as anti-inflammatory agents.

WO 00/07996 (Chiron Corporation) discloses pyrazole compounds for use as oestrogen receptor modulators which may be useful in, for example, the treatment of breast and endometrial cancers.

WO 2004/000318 (Cellular Genomics) discloses amino-subsituted moncyclic compounds as kinase modulators that may be useful in the treatment of cancers.

WO 03/062392 (Ceretek LLC) discloses aryl imidazole amides as EDG receptor modulators that may be usefuil in the treatment of cancers.

WO 01/68585 (Fujisawa) discloses a class of amides for use as 5-HT anatagonists. WO 97/40017 (Novo Nordisk) discloses a broad class of heterocyclic compounds for use as protein tyrosine phosphatase modulators.

### SUMMARY OF THE INVENTION

The invention provides compounds that have cyclin dependent kinase inhibiting or modulating activity and glycogen synthase kinase-3 (GSK3) inhibiting or modulating activity, and/or Aurora kinase inhibiting or modulating activity, and which it is envisaged will be useful in preventing or treating disease states or conditions mediated by the kinases.

Thus, for example, it is envisaged that the compounds of the invention will be useful in alleviating or reducing the incidence of cancer.

Accordingly, the invention provides inter alia:
The use of a compound of the formula (I) as defined herein for the manufacture of a medicament for the prophylaxis or treatment of a disease state or condition mediated by a cyclin dependent kinase or glycogen synthase kinase-3.
A method for the prophylaxis or treatment of a disease state or condition mediated by a cyclin dependent kinase or glycogen synthase kinase-3, which method comprises administering to a subject in need thereof a compound of the formula (I) as defined herein.
A method for alleviating or reducing the incidence of a disease state or condition mediated by a cyclin dependent kinase or glycogen synthase kinase-3, which method comprises administering to a subject in need thereof a compound of the formula (I) as defined herein.
A method for treating a disease or condition comprising or arising from abnormal cell growth in a mammal, which method comprises administering to the mammal a compound of the formula (I) as defined herein in an amount effective in inhibiting abnormal cell growth.
A method for alleviating or reducing the incidence of a disease or condition comprising or arising from abnormal cell growth in a mammal, which method comprises administering to the mammal a compound of the formula (I) as defined herein in an amount effective in inhibiting abnormal cell growth.
A method for treating a disease or condition comprising or arising from abnormal cell growth in a mammal, the method comprising administering to the mammal a compound of the formula (I) as defined herein in an amount effective to inhibit a cdk kinase (such as cdk1 or cdk2) or glycogen synthase kinase-3 activity.
A method for alleviating or reducing the incidence of a disease or condition comprising or arising from abnormal cell growth in a mammal, the method comprising administering to the mammal a compound of the formula (I) as defined herein in an amount effective to inhibit a cdk kinase (such as cdk1 or cdk2) or glycogen synthase kinase-3 activity.

A method of inhibiting a cyclin dependent kinase or glycogen synthase kinase-3, which method comprises contacting the kinase with a kinase-inhibiting compound of the formula (I) as defined herein.
A method of modulating a cellular process (for example cell division) by inhibiting the activity of a cyclin dependent kinase or glycogen synthase kinase-3 using a compound of the formula (I) as defined herein.
The use of a compound of the formula (I) as defined herein for the manufacture of a medicament for prophylaxis or treatment of a disease or condition characterised by up-regulation of an Aurora kinase (e.g. Aurora A kinase or Aurora B kinase).
The use of a compound of the formula (I) as defined herein for the manufacture of a medicament for the prophylaxis or treatment of a cancer, the cancer being one which is characterised by up-regulation of an Aurora kinase (e.g. Aurora A kinase or Aurora B kinase).
The use of a compound of the formula (I) as defmed herein for the manufacture of a medicament for the prophylaxis or treatment of cancer in a patient selected from a sub-population possessing the Ile31 variant of the Aurora A gene.
The use of a compound of the formula (I) as defined herein for the manufacture of a medicament for the prophylaxis or treatment of cancer in a patient who has been diagnosed as forming part of a sub-population possessing the Ile31 variant of the Aurora A gene.
A method for the prophylaxis or treatment of a disease or condition characterised by up-regulation of an Aurora kinase (e.g. Aurora A kinase or Aurora B kinase), the method comprising administering a compound of the formula (I) as defined herein.
A method for alleviating or reducing the incidence of a disease or condition characterised by up-regulation of an Aurora kinase (e.g. Aurora A kinase or Aurora B kinase), the method comprising administering a compound of the formula (I) as defined herein.
A method for the prophylaxis or treatment of (or alleviating or reducing the incidence of) cancer in a patient suffering from or suspected of suffering from cancer; which method comprises (i) subjecting a patient to a diagnostic test to determine whether the patient possesses the Ile31 variant of the Aurora A gene; and (ii) where the patient does possess the said variant, thereafter administering to the patient a compound of the formula (I) as defined herein having Aurora kinase inhibiting activity.
A method for the prophylaxis or treatment of (or alleviating or reducing the incidence of) a disease state or condition characterised by up-regulation of an Aurora kinase (e.g. Aurora A kinase or Aurora B kinase); which method comprises (i) subjecting a patient to a diagnostic test to detect a marker characteristic of up-regulation of the Aurora kinase and (ii) where the diagnostic test is indicative of up-regulation of Aurora kinase, thereafter administering to the patient a compound of the formula (I) as defined herein having Aurora kinase inhibiting activity.

The invention further provides:
A pharmaceutical composition comprising a novel compound of the formula (I) as hereinbefore defined and a pharmaceutically acceptable carrier.
A compound of the formula (I) for use in medicine.

The compounds of the invention are represented by the general formula (I): 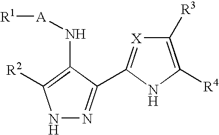
wherein
X is CR⁵ or N;
A is a bond or —(CH₂)ₘ—(B)ₙ—;
B is C═O, NR^{g}(C═O) or O(C═O) wherein R^{g} is hydrogen or C₁₋₄ hydrocarbyl optionally substituted by hydroxy or C₁₋₄ alkoxy;
m is 0, 1 or 2;
n is 0 or 1;
R¹ is hydrogen, a carbocyclic or heterocyclic group having from 3 to 12 ring members, or an optionally substituted C₁₋₈ hydrocarbyl group;
R² is hydrogen, halogen, methoxy, or a C₁₋₄ hydrocarbyl group optionally substituted by halogen, hydroxyl or methoxy;
R³ and R⁴ are the same or different and each is selected from hydrogen, CN, C(O)R⁸, optionally substituted C₁₋₈ hydrocarbyl and carbocyclic or heterocyclic groups having from 3 to 12 ring members; and
R⁵ is hydrogen, a group R² or a group R¹⁰ wherein R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
RC is selected from hydrogen and C₁₋₄ hydrocarbyl;
X¹ is O, S or NR^{c} and X² is ═O, ═S or ═NR^{c}; and
R⁸ is selected from OR¹¹, SR¹¹ and NR¹²R¹³;
R¹¹ is selected from optionally substituted C₁₋₈ hydrocarbyl and carbocyclic or
heterocyclic groups having from 3 to 12 ring members; and
one of R¹² and R¹³ is a group R¹¹ and the other of R¹² and R¹³ is hydrogen or C₁₋₄ alkyl; or R¹² and R¹³ and the nitrogen atom to which they are attached together form a saturated heterocyclic group having from 4 to 7 ring members and containing 1, 2 or 3 heteroatom ring members selected from N, O and S.
   General Preferences and Definitions

The aforementioned methods and uses, and any other therapeutic and diagnostic methods and uses, and methods of treating animals and plants defined herein, may also employ any sub-group, sub-genus, preference or example falling within formula (I), for example the compounds of formulae (II) to (IX) and any sub-groups thereof, uless the context indicates otherwise.

General Preferences and Definitions

The following general preferences and definitions shall apply to each of the moieties R¹ to R¹⁰, and their various sub-groups, sub-defmitions, examples and embodiments unless the context indicates otherwise. In this specification, a superscript letter following the number of an R group indicates that the R group is a sub-group of the R group designated solely by the number. Thus, for example R^{1a}, R^{1b} and R^{1c} are all sub groups of R¹, and, analogously, R^{9a} and R^{9b} are subgroups of R⁹. Thus, unless indicated otherwise, the general preferences, definitions and examples set out for, e.g. R¹ apply also to its sub-groups R^{1a}, R^{1b} R^{1c} etcetera, and similarly with the other R groups.

Any references to formula (I) herein shall also be taken to refer to formulae (II) to (VIII) and any other sub-group of compounds within formula (I) unless the context requires otherwise.

The term upregulation of Aurora kinase as used herein is defined as including elevated expression or over-expression of Aurora kinase, including gene amplification (i.e. multiple gene copies) and increased expression by a transcriptional effect, and hyperactivity and activation of Aurora kinase, including activation by mutations.

The term “substituent” as used herein refers to a moiety other than hydrogen, unless the context indicates otherwise.

References to “carbocyclic” and “heterocyclic” groups as used herein shall, unless the context indicates otherwise, include both aromatic and non-aromatic ring systems. Thus, for example, the term “carbocyclic and heterocyclic groups” includes within its scope aromatic, non-aromatic, unsaturated, partially saturated and fully saturated carbocyclic and heterocyclic ring systems. In general, such groups may be monocyclic or bicyclic and may contain, for example, 3 to 12 ring members, more usually 5 to 10 ring members. Examples of monocyclic groups are groups containing 3, 4, 5, 6, 7, and 8 ring members, more usually 3 to 7, and preferably 5 or 6 ring members. Examples of bicyclic groups are those containing 8, 9, 10, 11 and 12 ring members, and more usually 9 or 10 ring members.

The carbocyclic or heterocyclic groups can be aryl or heteroaryl groups having from 5 to 12 ring members, more usually from 5 to 10 ring members. The term “aryl” as used herein refers to a carbocyclic group having aromatic character and the term “heteroaryl” is used herein to denote a heterocyclic group having aromatic character. The terms “aryl” and “heteroaryl” embrace polycyclic (e.g. bicyclic) ring systems wherein one or more rings are non-aromatic, provided that at least one ring is aromatic. In such polycyclic systems, the group may be attached by the aromatic ring, or by a non-aromatic ring. The aryl or heteroaryl groups can be monocyclic or bicyclic groups and can be unsubstituted or 'substituted with one or more substituents, for example one or more groups R¹⁰ as defined herein.

The term “non-aromatic group” embraces unsaturated ring systems without aromatic character, partially saturated and fully saturated carbocyclic and heterocyclic ring systems. The terms “unsaturated” and “partially saturated” refer to rings wherein the ring structure(s) contains atoms sharing more than one valence bond i.e. the ring contains at least one multiple bond e.g. a C═C, C≡C or N═C bond. The term “fully saturated” refers to rings where there are no multiple bonds between ring atoms. Saturated carbocyclic groups include cycloalkyl groups as defined below. Partially saturated carbocyclic groups include cycloalkenyl groups as defined below, for example cyclopentenyl, cyclohexenyl, cycloheptenyl and cyclooctenyl.

Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings, or two fused five membered rings. Each ring may contain up to about four heteroatoms typically selected from nitrogen, sulphur and oxygen. Typically the heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a a 5- or 6-membered ring containing 1, 2 or 3 ring heteroatoms;
e) a pyrazole ring fused to a a 5- or 6-membered ring containing 1 or 2 ring heteroatoms;
f) an imidazole ring fused to a 5- or 6-membered ring containing 1 or 2 ring heteroatoms;
g) an oxazole ring fused to a 5- or 6-membered ring containing 1 or 2 ring heteroatoms;
h) an isoxazole ring fused to a 5- or 6-membered ring containing 1 or 2 ring heteroatoms;
i) a thiazole ring fused to a 5- or 6-membered ring containing 1 or 2 ring heteroatoms;
j) an isothiazole ring fused to a 5- or 6-membered ring containing 1 or 2 ring heteroatoms;
k) a thiophene ring fused to a 5- or 6-membered ring containing 1, 2 or 3 ring heteroatoms;
l) a furan ring fused to a 5- or 6-membered ring containing 1, 2 or 3 ring heteroatoms;
m) an oxazole ring fused to a 5- or 6-membered ring containing 1 or 2 ring heteroatoms;
n) an isoxazole ring fused to a 5- or 6-membered ring containing 1 or 2 ring heteroatoms;
o) a cyclohexyl ring fused to a 5- or 6-membered ring containing 1, 2 or 3 ring heteroatoms; and
p) a cyclopentyl ring fused to a 5- or 6-membered ring containing 1, 2 or 3 ring heteroatoms.

Particular examples of bicyclic heteroaryl groups containing a five membered ring fused to another five membered ring include but are not limited to iinidazothiazole (e.g. imidazo[2,1-b]thiazole) and imidazoimidazole (e.g. imidazo[1,2-a]imidazoie).

Particular examples of bicyclic heteroaryl groups containing a six membered ring fused to a five membered ring include but are not limited to benzfuran, benzthiophene, benzimidazole, benzoxazole, isobenzoxazole, benzisoxazole, benzthiazole, benzisothiazole, isobenzofuran, indole, isoindole, indolizine, indoline, isoindoline, purine (e.g., adenine, guanine), indazole, pyrazolopyrimidine (e.g. pyrazolo[1,5-a]pyrimidine), triazolopyrimidine (e.g. [1,2,4]triazolo[1,5-a]pyrimidine), benzodioxole and pyrazolopyridine (e.g. pyrazolo[15-a]pyridine) groups.

Particular examples of bicyclic heteroaryl groups containing two fused six membered rings include but are not limited to quinoline, isoquinoline, cbroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups.

Examples of polycyclic aryl and heteroaryl groups containing an aromatic ring and a non-aromatic ring include tetrahydronaphthalene, tetrahydroisoquinoline, tetrahydroquinoline, dihydrobenzthiene, dihydrobenzfuran, 2,3-dihydro-benzo[1,4]dioxine, benzo[1,3]dioxole, 4,5,6,7-tetrahydrobenzofuran, indoline and indane groups.

Examples of carbocyclic aryl groups include phenyl, naphthyl, indenyl, and tetrahydronaphthyl groups.

Examples of non-aromatic heterocyclic groups are groups having from 3 to 12 ring members, more usually 5 to 10 ring members. Such groups can be monocyclic or bicyclic, for example, and typically have from 1 to 5 heteroatom ring members (more usually 1, 2, 3 or 4 heteroatom ring members), usually selected from nitrogen, oxygen and sulphur. The heterocylic groups can contain, for example, cyclic ether moieties (e.g. as in tetrahydrofuran and dioxane), cyclic thioether moieties (e.g. as in tetrahydrothiophene and dithiane), cyclic amine moieties (e.g. as in pyrrolidine), cyclic amide moieties (e.g. as in pyrrolidone), cyclic thioamides, cyclic thioesters, cyclic ureas (e.g. as in imidazolidin-2-one) cyclic ester moieties (e.g. as in butyrolactone), cyclic sulphones (e.g. as in sulpholane and sulpholene), cyclic sulphoxides, cyclic sulphonamides and combinations thereof (e.g. thiomorpholine).

Particular examples include morpholine, piperidine (e.g. 1-piperidinyl, 2-piperidinyl, 3-piperidinyl and 4-piperidinyl), piperidone, pyrrolidine (e.g. 1-pyrrolidinyl, 2-pyrrolidinyl and 3-pyrrolidinyl), pyrrolidone, azetidine, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazone, piperazine, and N-alkyl piperazines such as N-methyl piperazine. In general, preferred non-aromatic heterocyclic groups include saturated groups such as piperidine, pyrrolidine, azetidine, morpholine, piperazine and N-alkyl piperazines.

Examples of non-aromatic carbocyclic groups include cycloalkane groups such as cyclohexyl and cyclopentyl, cycloalkenyl groups such as cyclopentenyl, cyclohexenyl, cycloheptenyl and cyclooctenyl, as well as cyclohexadienyl, cyclooctatetraene, tetrahydronaphthenyl and decalinyl.

Where reference is made herein to carbocyclic and heterocyclic groups, the carbocyclic or heterocyclic ring can, unless the context indicates otherwise, be unsubstituted or substituted by one or more substituent groups R¹⁰ selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}—R^{b} wherein Ra is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; or two adjacent groups R¹⁰, together with the carbon atoms or heteroatoms to which they are attached may form a 5-membered heteroaryl ring or a 5- or 6-membered non-aromatic carbocyclic or heterocyclic ring, wherein the said heteroaryl and heterocyclic groups contain up to 3 heteroatom ring members selected from N, O and S;
R^{c} is selected from hydrogen and C₁₋₄ hydrocarbyl; and

X¹ is O, S or NR^{c} and X² is ═O, ═S or ═NR^{c}.

Where the substituent group R¹⁰ comprises or includes a carbocyclic or heterocyclic group, the said carbocyclic or heterocyclic group may be unsubstituted or may itself be substituted with one or more furer substituent groups R¹⁰. In one sub-group of compounds of the formula (I), such further substituent groups R¹⁰ may include carbocyclic or heterocyclic groups, which are typically not themselves further substituted. In another sub-group of compounds of the formula (I), the said further substituents do not include carbocyclic or heterocyclic groups but are otherwise selected from the groups listed above in the defmition of R¹⁰.

The substituents R¹⁰ may be selected such that they contain no more than 20 non-hydrogen atoms, for example, no more than 15 non-hydrogen atoms, e.g. no more than 12, or 11, or 10, or 9, or 8, or 7, or 6, or 5 non-hydrogen atoms.

Where the carbocyclic and heterocyclic groups have a pair of substituents on adjacent ring atoms, the two substituents may be linked so as to form a cyclic group. For example, an adjacent pair of substituents on adjacent carbon atoms of a ring may be linked via one or more heteroatoms and optionally substituted alkylene groups to form a fused oxa-, dioxa-, aza-, diaza- or oxa-aza-cycloalkyl group. Examples of such linked substituent groups include: 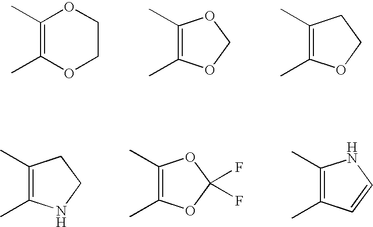
Examples of halogen substituents include fluorine, chlorine, bromine and iodine. Fluorine and chlorine are particularly preferred.

In the definition of the compounds of the formula (I) above and as used hereinafter, the term “hydrocarbyl” is a generic term encompassing aliphatic, alicyclic and aromatic groups having an all-carbon backbone, except where otherwise stated. In certain cases, as defined herein, one or more of the carbon atoms making up the carbon backbone may be replaced by a specified atom or group of atoms. Examples of hydrocarbyl groups include alkyl, cycloalkyl, cycloalkenyl, carbocyclic aryl, alkenyl, alkynyl, cycloalkylalkyl, cycloalkenylalkyl, and carbocyclic aralkyl, aralkenyl and aralkynyl groups. Such groups can be unsubstituted or, where stated, substituted by one or more substituents as defined herein. The examples and preferences expressed below apply to each of the hydrocarbyl substituent groups or hydrocarbyl-containing substituent groups referred to in the various definitions of substituents for compounds of the formula (I) unless the context indicates otherwise.

Preferred non-aromatic hydrocarbyl groups are saturated groups such as alkyl and cycloalkyl groups.

Generally by way of example, the hydrocarbyl groups can have up to eight carbon atoms, unless the context requires otherwise. Within the sub-set of hydrocarbyl groups having 1 to 8 carbon atoms, particular examples are C₁₋₆ hydrocarbyl groups, such as C₁₋₄ hydrocarbyl groups (e.g. C₁₋₃ hydrocarbyl groups or C₁₋₂ hydrocarbyl groups), specific examples being any individual value or combination of values selected from C₁, C₂, C₃, C₄, C₅, C₆, C₇ and C₈ hydrocarbyl groups.

The term “alkyl” covers both straight chain and branched chain alkyl groups. Examples of alkyl groups include methyl, ethyl, propyl, isopropyl, n-butyl, isobutyl, tert-butyl, n-pentyi, 2-pentyl, 3-pentyl, 2-methyl butyl, 3-methyl butyl, and n-hexyl and its isomers. Within the sub-set of alkyl groups having 1 to 8 carbon atoms, particular examples are C₁₋₆ alkyl groups, such as C₁₋₄ alkyl groups (e.g. C₁₋₃ alkyl groups or C₁₋₂ alkyl groups).

Examples of cycloalkyl groups are those derived from cyclopropane, cyclobutane, cyclopentane, cyclohexane and cycloheptane. Within the sub-set of cycloalkyl groups the cycloalkyl group will have from 3 to 8 carbon atoms, particular examples being C₃₋₆ cycloalkyl groups.

Examples of alkenyl groups include, but are not limited to, ethenyl (vinyl), 1-propenyl, 2-propenyl (allyl), isopropenyl, butenyl, buta-1,4-dienyl, pentenyl, and hexenyl. Within the sub-set of alkenyl groups the alkenyl group will have 2 to 8 carbon atoms, particular examples being C₂₋₆ alkenyl groups, such as C₂₋₄ alkenyl groups.

Examples of cycloalkenyl groups include, but are not limited to, cyclopropenyl, cyclobutenyl, cyclopentenyl, cyclopentadienyl and cyclohexenyl. Within the sub-set of cycloalkenyl groups the cycloalkenyl groups have from 3 to 8 carbon atoms, and particular examples are C₃₋₆ cycloalkenyl groups.

Examples of alkynyl groups include, but are not limited to, ethynyl and 2-propynyl (propargyl) groups. Within the sub-set of alkynyl groups having 2 to 8 carbon atoms, particular examples are C₂₋₆ alkynyl groups, such as C₂₋₄ alkynyl groups.

Examples of carbocyclic aryl groups include substituted and unsubstituted phenyl groups.

xamples of cycloalkylalkyl, cycloalkenylalkyl, carbocyclic aralkyl, aralkenyl and aralkynyl groups include phenethyl, benzyl, styryl, phenylethynyl, cyclohexylmethyl, cyclopentylmethyl, cyclobutylmethyl, cyclopropylmethyl and cyclopentenylmethyl groups.

When present, and where stated, a hydrocarbyl group can be optionally substituted by one or more substituents selected from hydroxy, oxo, alkoxy, carboxy, halogen, cyano, nitro, amino, mono- or di-C₁₋₄ hydrocarbylamino, and monocyclic or bicyclic carbocyclic and heterocyclic groups having from 3 to 12 (typically 3 to 10 and more usually 5 to 10) ring members. Preferred substituents include halogen such as fluorine. Thus, for example, the substituted hydrocarbyl group can be a partially fluorinated or perfluorinated group such as difluoromethyl or trifluoromethyl. In one embodiment preferred substituents include monocyclic carbocyclic and heterocyclic groups having 3-7 ring members, more usually 3, 4, 5 or 6 ring members.

Where stated, one or more carbon atoms of a hydrocarbyl group may optionally be replaced by O, S, SO, SO₂, NR^{c}, X¹C(X²), C(X²)X¹ or X¹C(X²)X¹ wherein X¹ and X² are as hereinbefore defined, provided that at least one carbon atom of the hydrocarbyl group remains. For example, 1, 2, 3 or 4 carbon atoms of the hydrocarbyl group may be replaced by one of the atoms or groups listed, and the replacing atoms or groups may be the same or different. In general, the number of linear or backbone carbon atoms replaced will correspond to the number of linear or backbone atoms in the group replacing them. Examples of groups in which one or more carbon atom of the hydrocarbyl group have been replaced by a replacement atom or group as defined above include ethers and thioethers (C replaced by O or S), amides, esters, thioamides and thioesters (C—C replaced by X¹C(X²) or C(X²)X¹), sulphones and sulphoxides (C replaced by SO or SO₂), amines (C replaced by NR^{c}), and ureas, carbonates and carbamates (C—C—C replaced by X¹C(X²)X¹).

Where an amino group has two hydrocarbyl substituents, they may, together with the nitrogen atom to which they are attached, and optionally with another heteroatom such as nitrogen, sulphur, or oxygen, link to form a ring structure of 4 to 7 ring members.

The definition “R^{a}—R^{b}” as used herein, either with regard to substituents present on a carbocyclic or heterocyclic moiety, or with regard to other substituents present at other locations on the compounds of the formula (I), includes inter alia compounds wherein R^{a} is selected from a bond, O, CO, OC(O), SC(O), NR^{c}C(O), OC(S), SC(S), NR^{c}C(S), OC(NR^{c}), SC(NR^{c})NR^{c}C(NR^{C}) C(O)O, C(O)S, C(O)NR^{c}, C(S)O, C(S)S, C(S)NR^{c}, C(NR^{c})O, C(NR^{c})S, C(NR^{c})NR^{c}, OC(O)O, SC(O)O, NR^{c}C(O)O, OC(S)O, SC(S)O, NR^{c}C(S)O, OC(NR^{c})O, SC(NR^{c})O, NR^{c}(NR^{c})O, OC(O)S, SC(O)S, NR^{c}C(O)S, OC(S)S, SC(S)S, NR^{c}C(S)S, OC(NR^{c})S, SC(NR^{c})S, NR^{c}C(NR^{c})S, OC(O)NR^{c}, SC(O)NR^{c}, NR^{c}C(O)NR^{c}, OC(S)NR^{c}, SC(S)NR^{c}, NR^{c}C(S)NR^{c}, OC(NR^{c})NR^{c}, SC(NR^{c})NR^{c}, NR^{c}C(NR^{c}NR^{c}, S, SO, SO₂, NR^{c}, SO₂NR^{c} and NR^{c}SO₂ wherein R^{c} is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined. Examples of hydrocarbyl, carbocyclic and heterocyclic groups are as set out above.

When R^{a} is O and R^{b} is a C₁₋₈ hydrocarbyl group, R^{a} and R^{b} together form a hydrocarbyloxy group. Preferred hydrocarbyloxy groups include saturated hydrocarbyloxy such as alkoxy (e.g. C₁₋₆ alkoxy, more usually C₁₋₄ alkoxy such as ethoxy and methoxy, particularly methoxy), cycloalkoxy (e.g. C₃₋₆ cycloalkoxy such as cyclopropyloxy, cyclobutyloxy, cyclopentyloxy and cyclohexyloxy) and cycloalkyalkoxy (e.g. C₃₋₆ cycloalkyl-C₁₋₂ alkoxy such as cyclopropylmethoxy).

The hydrocarbyloxy groups can be substituted by various substituents as defined herein. For example, the alkoxy groups can be substituted by halogen (e.g. as in difluoromethoxy and trifluoromethoxy), hydroxy (e.g. as in hydroxyethoxy), C₁₋₂ alkoxy (e.g. as in methoxyethoxy), hydroxy-C₁₋₂ alkyl (as in hydroxyethoxyethoxy) or a cyclic group (e.g. a cycloalkyl group or non-aromatic heterocyclic group as hereinbefore defined). Examples of alkoxy groups bearing a non-aromatic heterocyclic group as a substituent are those in which the heterocyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkoxy group is a C₁₋₄ alkoxy group, more typically a C₁₋₃ alkoxy group such as methoxy, ethoxy or n-propoxy.

Alkoxy groups substituted by a monocyclic group such as pyrrolidine, piperidine, morpholine and piperazine and N-substituted derivatives thereof such as N-benzyl, N—C₁₋₄ acyl and N—C₁₄ alkoxycarbonyl. Particular examples include pyrrolidinoethoxy, piperidinoethoxy and piperazinoethoxy.

When R^{a} is a bond and R^{b} is a C₁₋₈ hydrocarbyl group, examples of hydrocarbyl groups R^{a}—R^{b} are as hereinbefore defined. The hydrocarbyl groups may be saturated groups such as cycloalkyl and alkyl and particular examples of such groups include methyl, ethyl and cyclopropyl. The hydrocarbyl (e.g. alkyl) groups can be substituted by various groups and atoms as defined herein. Examples of substituted alkyl groups include alkyl groups substituted by one or more halogen atoms such as fluorine and chlorine particular examples including bromoethyl, chloroethyl and trifluoromethyl), or hydroxy (e.g. hydroxymethyl and hydroxyethyl), C₁₋₈ acyloxy (e.g. acetoxymethyl and benzyloxymethyl), amino and mono- and dialkylamino (e.g. aminoethyl, methylaminoethyl, dimethylaminomethyl, dimethylaminoethyl and tert-butylaminomethyl), alkoxy (e.g. C₁₋₂ alkoxy such as methoxy—as in methoxyethyl), and cyclic groups such as cycloalkyl groups, aryl groups, heteroaryl groups and non-aromatic heterocyclic groups as hereinbefore defined).

Particular examples of alkyl groups substituted by a cyclic group are those wherein the cyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkyl group is a C₁₋₄ alkyl group, more typically a C₁₋₃ alkyl group such as methyl, ethyl or n-propyl. Specific examples of alkyl groups substituted by a cyclic group include pyrrolidinomethyl, pyrrolidinopropyl, morpholinomethyl, morpholinoethyl, morpholinopropyl, piperidinylmethyl, piperazinomethyl and N-substituted forms thereof as defmed herein.

Particular examples of alkyl groups substituted by aryl groups and heteroaryl groups include benzyl and pyridylmethyl groups.

When R^{a} is SO₂NR^{c}, R^{b} can be, for example, hydrogen or an optionally substituted C₁₋₈ hydrocarbyl group, or a carbocyclic or heterocyclic group. Examples of R^{a}—R^{b}where R^{a} is SO₂NR^{c} include aminosulphonyl, C₁₋₄ alkylaminosulphonyl and di-C₁₋₄ alkylaminosulphonyl groups, and sulphonamides formed from a cyclic amino group such as piperidine, morpholine, pyrrolidine, or an optionally N-substituted piperazine such as N-methyl piperazine.

Examples of groups R^{a}—R^{b} where R^{a} is SO₂ include alkylsulphonyl, heteroarylsulphonyl and arylsulphonyl groups, particularly monocyclic aryl and heteroaryl sulphonyl groups. Particular examples include methylsulphonyl, phenylsulphonyl and toluenesulphonyl.

When R^{a} is NR^{c}, R^{b} can be, for example, hydrogen or an optionally substituted C₁₋₈ hydrocarbyl group, or a carbocyclic or heterocyclic group. Examples of R^{a}—R^{b} where R^{a} is NR^{c} include amino, C₁₋₄ alkylamino (e.g. methylamino, ethylamino, propylamino, isopropylamino, tert-butylamino), di-C₁₋₄ alkylamino (e.g. dimethylamino and diethylamino) and cycloalkylamino (e.g. cyclopropylamino, cyclopentylamino and cyclohexylamino).

Specific Embodiments of and Preferences for X and R¹ to R¹³

In formula (I), X can be CR⁵ or N. In one particular embodiment, X is N. In another particular embodiment, X is CH. Preferably X is N.

Where R⁵ is other than hydrogen, it is preferably a small substituent containing no more than 14 atoms, for example a C₁₋₄ alkyl or cycloalkyl group such as methyl, ethyl, propyl and butyl, or cyclopropyl and cyclobutyl.

A is a bond or —(CH₂)ₘ—(B)ₙ— wherein B is C═O, NR^{g}(C═O) or O(C═O), m is 0, 1 or 2; and n is 0 or 1. In one preferred group of compounds of the invention, m is 0 or 1, n is 1 and B is C═O or NR^{g}(C═O), preferably C═O. More preferably, m is 0, n is 1 and B is C═O. It is presently preferred that when B is NR^{g}(C═O), R^{g} is hydrogen.

It will be appreciated that the moiety R¹-A-NH linked to the 4-position of the pyrazole ring can take the form of an amine R¹—(CH₂)ₘ—NH, an amide R¹—(CH₂)ₘ—C(═O)NH, a urea R¹—(CH₂)ₘ—NHC(═O)NH or a carbamate R¹—(CH₂)ₘ—OC(═O)NH wherein in each case m is 0, 1 or 2, preferably 0 or 1 and most preferably 0.

R¹ is hydrogen, a carbocyclic or heterocyclic group having from 3 to 12 ring members, or an optionally substituted C₁₋₈ hydrocarbyl group. Examples of carbocyclic or heterocyclic groups, and optionally substituted hydrocarbyl groups are as set out above.

For example, R¹ can be a monocyclic or bicyclic group having from 3 to 10 ring members.

Where R¹ is a monocyclic group, typically it has 3 to 7 ring members, more usually 3 to 6 ring members, for example, 3, 4, 5 or 6.

When the monocyclic group R¹ is an aryl group, it will have 6 ring members and will be an unsubstituted or subituted phenyl ring.

When the monocyclic group R¹ is a non-aromatic carbocyclic group, it can have from 3 to 7 ring members, more usually 3 to 6 ring members, for example, 3, or 4, or 5, or 6 ring members. The non-aromatic carbocyclic group may be saturated or partially unsaturated but preferably it is saturated, i.e. R¹ is a cycloalkyl group.

When the monocyclic group R¹ is a heteroaryl group, it will have 5 or 6 ring members. Examples of heteroaryl groups having 5 and 6 ring members are set out above, and particular examples are described below.

In one sub-group of compounds, the heteroaryl group has 5 ring members.

In another sub-group of compounds, the heteroaryl group has 6 ring members. The monocyclic heteroaryl groups R¹ typically have up to 4 ring heteroatoms selected from N, O and S, and more typically up to 3 ring heteroatoms, for example 1, or 2, or 3 ring heteroatoms.

When R¹ is a non-aromatic monocyclic heterocyclic group, it may be any one of the groups listed hereinabove or hereinafter. Such groups typically have from 4 to 7 ring members and more preferably 5 or 6 ring members. The non-aromatic monocyclic heterocyclic groups typically contain up to 3 ring heteroatoms, more usually 1 or 2 ring heteroatoms, selected from N, S and O. The heterocyclic group may be saturated or partially unsaturated, but preferably it is saturated. Particular examples of non-aromatic monocyclic heterocyclic groups are the particular and preferred examples defmed in the “General Preferences and Definitions” section above, and as set out in the tables and examples below.

Where R¹ is a bicyclic group, typically it has 8 to 10 ring members, for example 8, or 9, or 10 ring members. The bicyclic group can be an aryl or heteroaryl group and examples of such groups include groups comprising a 5-membered ring fused to another 5-membered ring; a 5-membered ring fused to a 6-membered ring; and a 6-membered ring fused to another 6-membered ring. Examples of groups in each of these categories are set out above in the “General Preferences and Definitions” section.

A bicyclic aryl or heteroaryl group can comprise two aromatic or unsaturated rings, or one aromatic and one non-aromatic (e.g. partially saturated) ring. Bicyclic heteroaryl groups typically contain up to 4 heteroatom ring members selected from N, S and O. Thus, for example, they may contain 1, or 2, or 3, or 4 heteroatom ring members.

In one general embodiment, R¹ may other than an indazole.

In the monocyclic and bicyclic heterocyclic groups R¹, examples of combinations of heteroatom ring members include N; NN; NNN; NNNN; NO; NNO; NS, NNS, O, S, OO and SS.

Particular examples of R¹ include aryl and heteroaryl groups selected from phenyl, pyrazolo[1,5-a]pyridinyl (e.g. pyrazolo[1,5-a]pyridin-3-yl), furanyl (e.g. 2-furanyl and 3-furanyl), indolyl (e.g. 3-indolyl, 4-indolyl and 7-indolyl), oxazolyl, thiazolyl (e.g. thiazol-2-yl and thiazol-5-yl), isoxazolyl (e.g. isoxazol-3-yl and isoxazol-4-yl), pyrrolyl (e.g. 3-pyrrolyl), pyridyl (e.g. 2-pyridyl), quinolinyl (e.g. quinolin-8-yl), 2,3-dihydro-benzo[1,4]dioxine (e.g. 2,3-dihydro-benzo[1,4]dioxin-5-yl), benzo[1,3]dioxole (e.g. benzo[1,3]dioxol-4-yl), 2,3-dihydrobenzofuranyl (e.g. 2,3-dihydrobenzofuran-7-yl), imidazolyl and thienyl (e.g. 3-thienyl).

Preferred R¹ aryl and heteroaryl groups include phenyl, pyrazolo[1,5-a]pyridinyl, furanyl, 2,3-dihydrobenzofuranyl, thienyl, indolyl, thiazolyl, isoxazolyl and 2,3-dihydro-benzo[1,4]dioxine groups.

Preferred non-aromatic groups R¹ include monocyclic cycloalkyl and azacycloalkyl groups such as cyclohexyl, cyclopentyl and piperidinyl, particularly cyclohexyl and 4-piperidinyl groups.

Preferred substituted and unsubstituted C₁₋₈ hydrocarbyl groups include trifluoromethyl and tertiary butyl groups.

The group R¹ can be an unsubstituted or substituted carbocyclic or heterocyclic group in which one or more substituents can be selected from the group R¹⁰ as hereinbefore defined. In one embodiment, the substituents on R¹ may be selected from the group R^{10a} consisting of halogen, hydroxy, trifluoromethyl, cyano, nitro, carboxy, heterocyclic groups having 5 or 6 ring members and up to 2 heteroatoms selected from O, N and S, a group R^{a}—R^{b} wherein R^{a} is a bond, O, CO, X³C(X⁴), C(X⁴)X³, X³C(X⁴)X³, S, SO, or SO₂, and R^{b} is selected from hydrogen, heterocyclic groups having 5 or 6 ring members and up to 2 heteroatoms selected from O, N and S, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having 5 or 6 ring members and up to 2 heteroatoms selected from O, N and S; wherein one or more carbon atoms of the C₁₋₈ hydrocarbyl group may optionally be replaced by O, S, SO, SO₂, X³C(⁴), (X⁴)X³ or X³C(X⁴)X³; X³ is O or S; and X⁴ is ═O or ═S.

In a further embodiment, the substituents on R¹ may be selected from the group R^{10b} consisting of halogen, hydroxy, trifluoromethyl, cyano, nitro, carboxy, a group R^{a}—R^{b} wherein R^{a} is a bond, O, CO, X³C(X⁴), C(X⁴)X³, X³C(X⁴)X³, S, SO, or SO₂, and R^{b} is selected from hydrogen and a C₁₋₈ hydrocarbyl group optionally substituted by one or more substituents selected from hydroxy, oxo, halogen, cyano, nitro, carboxy; wherein one or more carbon atoms of the C₁₋₈ hydrocarbyl group may optionally be replaced by O, S, SO, SO₂, X³C(X⁴), C(X⁴)X³ or X³C(X⁴)X³; X³ is O or S; and X⁴is ═O or ═S.

In another embodiment, the substituents on R¹ may be selected from halogen, hydroxy, trifluoromethyl, a group R^{a}—R^{b} wherein R^{a} is a bond or O, and R^{b} is selected from hydrogen and a C₁₋₄ hydrocarbyl group optionally substituted by one or more substituents selected from hydroxyl and halogen.

Particular examples of substituents that may be present on a group R¹ (e.g. an aryl or heteroaryl group R¹) include fluorine, chlorine, methoxy, methyl, oxazolyl, morpholino, trifluoromethyl, pyrrolidino, pyrrolidinylethoxy, pyrrolidinylmethyl, difluoromethoxy and morpholinomethyl.

In one general embodiment, a carbocyclic or heterocyclic group R¹ may bear one or more acyclic substituents and no cyclic substituents.

In another general embodiment, a carbocyclic or heterocyclic group R¹ may bear one or more acyclic substituents and no cyclic substituents attached directly to the said carbocyclic or heterocyclic group.

The moiety R¹ may be substituted by more than one substituent. Thus, for example, there may be 1 or 2 or 3 or 4 substituents, and preferably up to 3 substituents. In one embodiment, where R¹ is a six membered ring (e.g. a carbocyclic ring such as a phenyl ring), there may be a single substituent which may be located at the 2-, 3- or 4-positions of the ring. In another embodiment, there may be two or three substituents and these may be located at the 2-, 3-, 4- or 6-positions around the ring. By way of example, a phenyl group R¹ may be 2,6-disubstituted, 2,3-disubstituted, 2,4-disubstituted 2,5-dis-ubstituted, 2,3,6-tisubstituted or 2,4,6-trisubstituted. More particularly, a phenyl group R¹ may be disubstituted at positions 2- and 6- with 5 substituents selected from fluorine, chlorine and R^{a}—R^{b}, where R¹ is a bond or O and R^{b} is C₁₋₄ alkyl, with fluorine being a particular substituent.

Particular examples of groups R¹ include the groups A1 to A60 set out in Table 1 below.

TABLE 1

| | |
|---|---|
| | A1 |
| 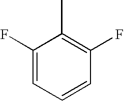 | |
| 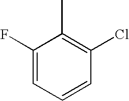 | A2 |
| 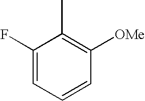 | A3 |
| 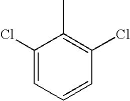 | A4 |
| 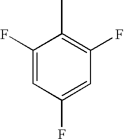 | A5 |
| 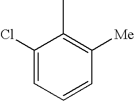 | A6 |
| 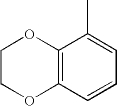 | A7 |
| 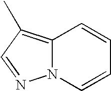 | A8 |
| 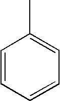 | A9 |

| | |
|---|---|
| | A10 |
| 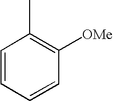 | |
| 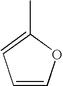 | A11 |
| 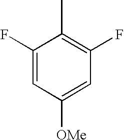 | A12 |
| 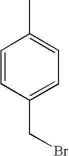 | A13 |
| 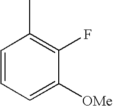 | A14 |
| 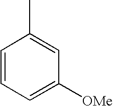 | A15 |
| 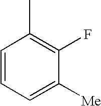 | A16 |
| 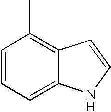 | A17 |
| 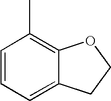 | A18 |
| 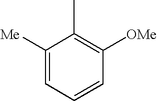 | A19 |
| 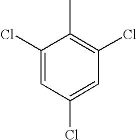 | A20 |
| 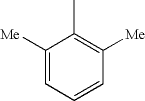 | A21 |
| 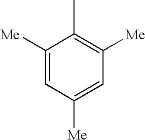 | A22 |
| 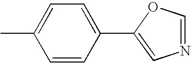 | A23 |
| 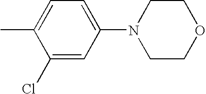 | A24 |
| 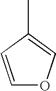 | A25 |
| 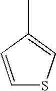 | A26 |
| 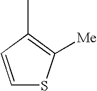 | A27 |
| 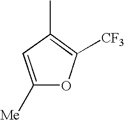 | A28 |
| 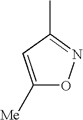 | A29 |
| 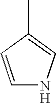 | A30 |
| 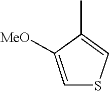 | A31 |
| 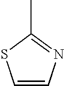 | A32 |
| 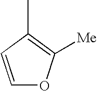 | A33 |
| 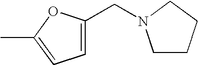 | A34 |
| 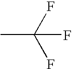 | A35 |
| 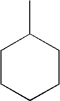 | A36 |
| 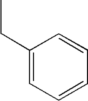 | A37 |
| 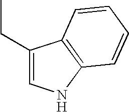 | A38 |
| 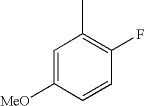 | A39 |
| 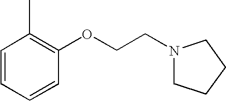 | A40 |
| 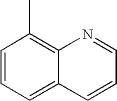 | A41 |
| 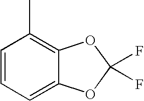 | A42 |
| 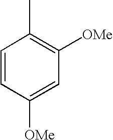 | A43 |
| 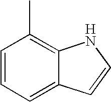 | A44 |
| 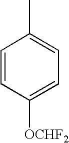 | A45 |
| 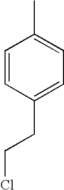 | A46 |
| 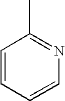 | A47 |
| 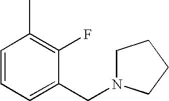 | A48 |
| 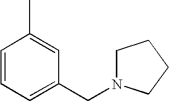 | A49 |
| 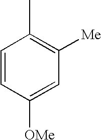 | A50 |
| 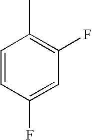 | A51 |
| 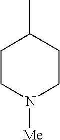 | A52 |
| 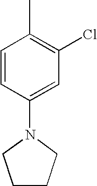 | A53 |
| 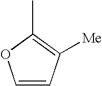 | A54 |
| 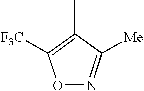 | A55 |
| 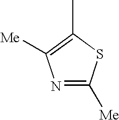 | A56 |
| 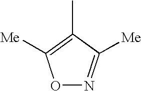 | A57 |
| 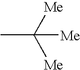 | A58 |
| 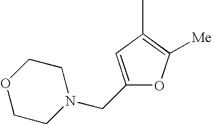 | A59 |
| 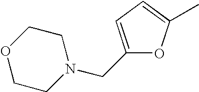 | A60 |
| 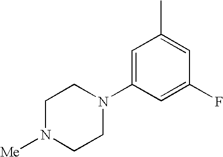 | A61 |
| 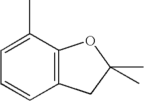 | A62 |
| 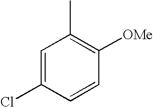 | A63 |

Preferred groups R¹ include groups A1 to A12 and A14 to A34.

A further preferred group is A61.

Particularly preferred groups are A1, A3, A61, A62 and A63.

Particularly preferred groups R¹ include 2,6-difluorophenyl, 2-chloro-6-fluorophenyl, 2-fluoro-6-methoxyphenyl, 2,6-dichlorophenyl, 2,4,6-trifluorophenyl, 2-chloro-6-methyl, 2,3-dihydro-benzo[1,4]dioxin-5-yl and pyrazolo[1,5-a]pyridin-3-yl.

A currently most preferred group R¹ is 2,6-difluorophenyl.

R² is hydrogen, halogen, methoxy, or a C₁₋₄ hydrocarbyl group optionally substituted by halogen, hydroxyl or methoxy. Preferably R² is hydrogen, chlorine or methyl, and most preferably R² is hydrogen.

In formula (I), R³ and R⁴ are the same or different and each is selected from hydrogen, CN, C(O)R⁸, optionally substituted C₁₋₈ hydrocarbyl and carbocyclic or heterocyclic groups having from 3 to 12 ring members. Examples of carbocyclic or heterocyclic groups, and optionally substituted hydrocarbyl groups are set out above.

In one embodiment, R³ and R⁴ are the same or different and each is selected from hydrogen, optionally substituted C₁₋₈ hydrocarbyl and carbocyclic or heterocyclic groups having from 3 to 12 ring members.

Where either one of R³ or R⁴ is a heterocyclic group directly attached to the imidazole ring, typically it is attached to the imidazole ring via a carbon atom of the heterocyclic group.

In one embodiment, R³ and R⁴ are the same or different and each is selected from hydrogen, optionally substituted C₁₋₈ hydrocarbyl and carbocyclic or heterocyclic groups having from 3 to 12 ring members.

The group R⁸ is selected from OR¹¹, SR¹¹ and NR¹²R¹³ and hence the moiety C(O)R⁸ can be an ester, thioester or amide. The group R¹¹ is selected from optionally substituted C₁₋₈ hydrocarbyl and carbocyclic or heterocyclic groups having from 3 to 12 ring members, whrein the hydrocarbyl, carbocyclic and heterocyclic groups can be as set out in the “General Preferences and Definitions” section above. Preferred groups R¹¹ include C₁₋₆ alkyl groups optionally substituted by one or more substituents selected from halogen, hydroxy, amino, mono- or di-C₁₋₄ alkylamino and C₁₋₄ alkoxy.

One of the groups R¹² and R¹³ is a group R¹¹ and the other of R¹² and R¹³ is hydrogen or C₁₋₄ alkyl; or R¹² and R¹³ and the nitrogen atom to which they are attached together form a saturated heterocyclic group having from 4 to 7 ring members and containing 1, 2 or 3 heteroatom ring members selected from N, O and S.

In one preferred embodiment, R¹² and R¹³ and the nitrogen atom to which they are attached together form a saturated heterocyclic group having from 4 to 7 ring members (more preferably 5 or 6 ring members) and containing 1 or 2 heteroatom ring members selected from N, O and S. Examples of saturated heterocyclic groups are set out above. Preferred heterocyclic groups include piperidino, piperazino, N-C₁₋₄alkylpiperazino (e.g. N-methyl piperazino) and morpholino.

In another preferred embodiment, R³ and R⁴ groups include optionally substituted C₁₋₈ hydrocarbyl, phenyl, naphthyl, thienyl, isoxazolyl, pyridyl, 2,3-dihydro-benzo[1,4]dioxine, cyano and CONR¹²R¹³, where NR¹²R¹³ is a saturated heterocyclic group as defmed herein.

In another embodiment, preferred R³ and R⁴ groups include optionally substituted C₁₋₈ hydrocarbyl, phenyl, naphthyl, thienyl, isoxazolyl, pyridyl, 2,3-dihydro-benzo[1,4]dioxine, with phenyl and pyridyl being particularly preferred.

It is preferred that at least one of R³ and R⁴ is other than hydrogen. In one sub-group of compounds of the formula (I), one of R³ and R⁴ is hydrogen, and the other is selected from optionally substituted C₁₋₈ hydrocarbyl and carbocyclic or heterocyclic groups having from 3 to 12 ring members.

In another sub-group of compounds, each of R³ and R⁴ is an optionally substituted group selected from C₁₋₈ hydrocarbyl, phenyl, naphthyl, thienyl, isoxazolyl, pyridyl, 2,3-dihydro-benzo[1,4]dioxine.

For example, in one embodiment, one of R³ and R⁴ is an optionally substituted group selected from phenyl, naphthyl, thienyl, isoxazolyl, pyridyl, 2,3-dihydro-benzo[1,4]dioxine, and the other one of R³ and R⁴ is an optionally substituted C₁₋₈ hydrocarbyl group.

In another embodiment, one of R³ and R⁴ is an optionally substituted group selected from phenyl, naphthyl, thienyl, isoxazolyl, pyridyl, 2,3-dihydro-benzo[1,4]dioxine and the other one of R³ and R⁴ is a group CONR¹²R¹³.

Where either one of R³ and R⁴ is an optionally substituted C₁₋₈ hydrocarbyl group, it can be, for example, optionally substituted by a substituent selected from optionally substituted monocyclic carbocyclic and heterocyclic groups, NR ²R ³, C₁₋₄ alkoxy, halogen, hydroxy, C₁₋₄ alkylsulphonylamino, amino, mono- and di-C₁₋₄ alkylamino, wherein the alkyl residues of the C₁₋₄ alkoxy, mono- and di-C₁₋₄ alkylamino groups may themselves be further substituted by a substituent selected from NR¹²R¹³, C₁₋₄ alkoxy, hydroxy, C₁₋₄ alkylsulphonylamino, amino, and mono- and di-C₁₋₄ alkylamino, wherein R¹² and R¹³ are as defined herein, and wherein the optional substituents for the carbocyclic and heterocyclic groups are selected from the group R¹⁰ and sub-groups thereof as defined herein.

In one embodiment, when either one of R³ and R⁴ is an optionally substituted C₁₋₈ hydrocarbyl group, it can be, for example, selected from a C₁₋₄ alkyl, hydroxy-C₁₋₄ alkyl or C₂₋₄ alkenyl group.

In another embodiment, when either one of R³ and R⁴ is an optionally substituted C₁₋₈ hydrocarbyl group, the C₁₋₈ hydrocarbyl group can be a C₁₋₄ alkyl group optionally substituted by a substituent selected from CONR¹²R¹³, C₁₋₄ alkoxy, halogen, hydroxy, C₁₋₄ alkylsulphonylamino, amino, mono- and di-C₁₋₄ alkylamino, wherein the alkyl residues of the C₁₋₄ alkoxy, mono- and di-C₁₋₄ alkylamino groups may themselves be further substituted by a substituent selected from CONR²R¹³, C₁₋₄ alkoxy, hydroxy, C₁₋₄ alkylsulphonylamino, amino, and mono- and di-C₁₋₄ alkylamino.

When R³ and R⁴ groups are carbocyclic or heterocyclic groups having from 3 to 12 ring members, they may be unsubstituted or substituted by one or more substituents selected from the groups R¹⁰, R^{10a} and R^{10b} and sub-groups thereof as hereinbefore defined.

In one embodiment, one of R³ and R⁴ is an unsubstituted pyridyl group.

In another embodiment, one of R³ and R⁴ is an unsubstituted phenyl group or a phenyl group substituted with up to 3 fluorine atoms.

In another embodiment, one of R³ and R⁴ is a morpholinomethyl group.

In a further embodiment, one of R³ and R⁴ is a morpholinocarbonyl group.

In another embodiment, one of R³ and R⁴ is a C₁₋₄alkyl group bearing a substituent selected from mono- and di-C₁₋₄alkylamino, hydroxy, C₁₋₄ alkylsulphonylamino, C₁₋₄ alkoxy, C₁₋₂ alkoxy-C₁₋₄alkylamino, and mono- and di-C₁₋₂alkylamino-C₁₋₄alkylamino.

In a further embodiment, R³ and R⁴ are the same or different and are selected from C₁₋₄alkyl groups optionally substituted by halogen, hydroxy or methoxy, preferably a halogen such as fluorine. A preferred halogen-substituted alkyl group is trifluoromethyl. More preferably, one of R³ and R⁴ is trifluoromethyl, methyl, ethyl, isopropyl or tert- butyl and the other is methyl.

Particular examples of the imidazole ring and substituents R³ and R⁴ are shown in Table 2 below.

TABLE 2
Examples of the Imidazole Group

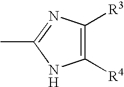

| | |
|---|---|
| | B1 |
| 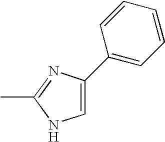 | |
| 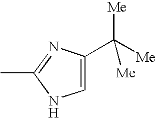 | B2 |
| 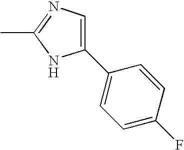 | B3 |
| 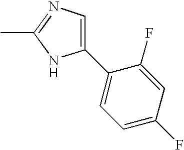 | B4 |
| 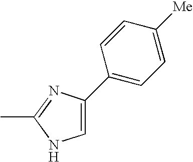 | B5 |
| 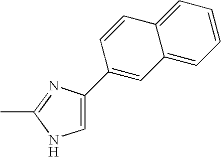 | B6 |
| 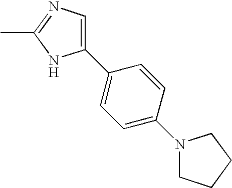 | B7 |
| 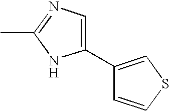 | B8 |
| 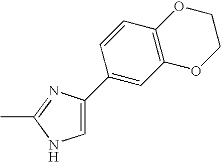 | B9 |

| | |
|---|---|
| | B10 |
| 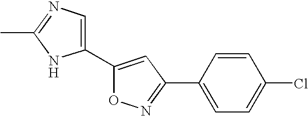 | |
| 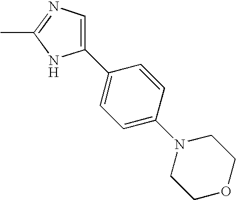 | B11 |
| 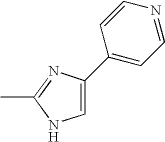 | B12 |
| 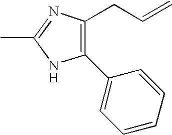 | B13 |
| 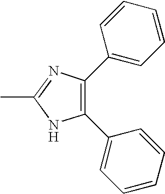 | B14 |
| 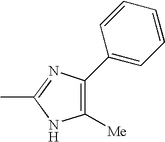 | B15 |
| 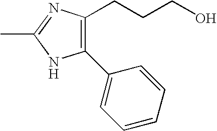 | B16 |
| 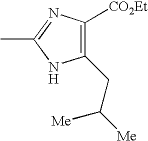 | B17 |
| 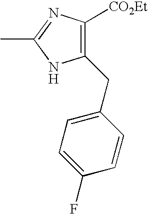 | B18 |
| 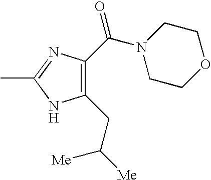 | B19 |
| 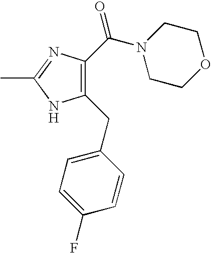 | B20 |
| 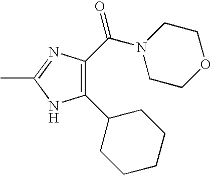 | B21 |
| 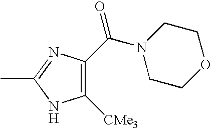 | B22 |
| 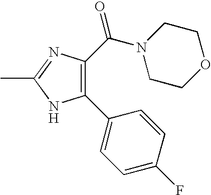 | B23 |
| 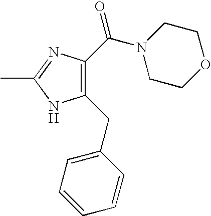 | B24 |
| 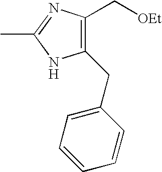 | B25 |
| 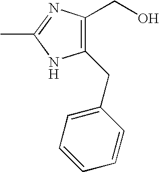 | B26 |
| 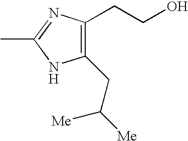 | B27 |
| 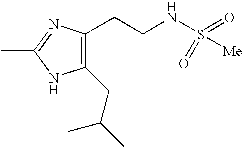 | B28 |
| 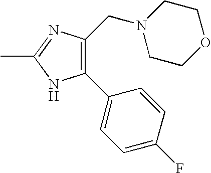 | B29 |
| 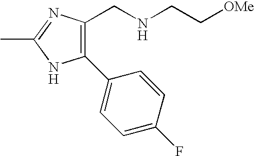 | B30 |
| 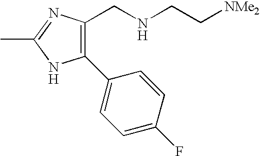 | B31 |
| 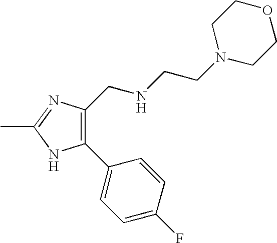 | B32 |
| 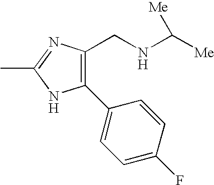 | B33 |
| 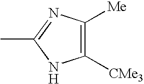 | B34 |
| 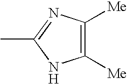 | B35 |
| 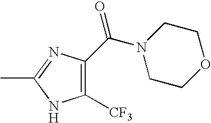 | B36 |
| 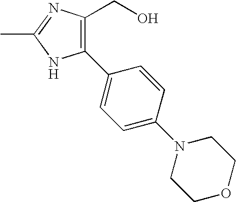 | B37 |
| 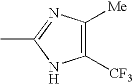 | B38 |
| 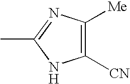 | B39 |
| 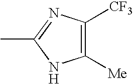 | B40 |
| 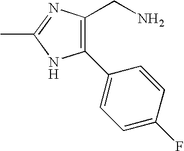 | B41 |

Preferred examples of the imidazole group are (i) the groups B1 to B6, B8, B9 and B11 to B16, and (ii) the groups B18, B19, B20, B22, B24, B25, B26, B27, B28, B29, B31, B34, B35, B37, B38 and B39.

In one preferred embodiment, the imidazole group is selected from groups B1 to B6, B8, B9, B11 to B13, B15 and B16.

In one preferred embodiment, the imidazole group is selected from groups B2, B4, B12, B15 and B16.

In a further preferred embodiment, the imidazole group is selected from B34, B35, B38 and B39.

For the avoidance of doubt, it is to be understood that each general and specific preference, embodiment and example of the groups R¹ may be combined with each general and specific preference, embodiment and example of the groups R² and/or R³ and/or R⁴ and/or R⁵ and/or R¹⁰ and that all such combinations are embraced by this application.

The various functional groups and substituents making up the compounds of the formula (I) are typically chosen such that the molecular weight of the compound of the formula (I) does-not exceed 1000. More usually, the molecular weight of the compound will be less than 750, for example less than 700, or less than 650, or less than 600, or less than 550. More preferably, the molecular weight is less than 525 and, for example, is 500 or less.

Particular compounds of the invention are as illustrated in the examples below.

Unless otherwise specified, a reference to a particular compound also includes ionic, salt, solvate, and protected forms thereof, for example, as discussed below.

Many compounds of the formula (I) can exist in the form of salts, for example acid addition salts or, in certain cases salts of organic and inorganic bases such as carboxylate, sulphonate and phosphate salts. All such salts are within the scope of this invention, and references to compounds of the formula (I) include the salt forms of the compounds.

Acid addition salts may be formed with a wide variety of acids, both inorganic and organic. Examples of acid addition salts include salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth metal cations such as Ca²⁺ and Mg²⁺, and other cations such as Al³+. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium salts are within the scope of formula (I).

The salt forms of the compounds of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

Compounds of the formula (I) containing an amine finction may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistiy, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formula may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

For example, in compounds of the formula (I) where X is N, the imidazole group may take either of the following two tautomeric forms A and B. For simplicity, the general formula (I) illustrates form A but the formula is to be taken as embracing both tautomeric forms.

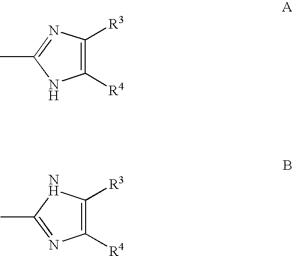

The pyrazole ring may also exhibit tautomerism and can exist in the two tautomeric forms C and D below.

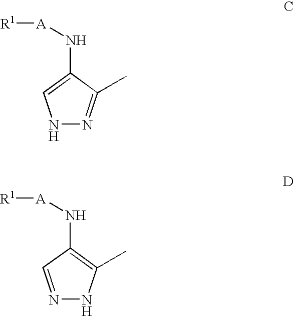

The general formula (I) illustrates form C but the formula is to be taken as embracing both form C and form D.

Other examples of tautomeric forms include, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

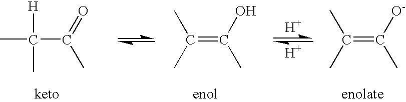

Where compounds of the formula (I) contain one or more chiral centres, and can exist in the form of two or more optical isomers, references to compounds of the formula (I) include all optical isomeric forms thereof (e.g. enantiomers, epimers and diastereoisomers), either as individual optical isomers, or mixtures or two or more optical isomers, unless the context requires otherwise.

For example, the group A can include one or more chiral centres. Thus, when E and R¹ are both attached to the same carbon atom on the linker group A, the said carbon atom is typically chiral and hence the compound of the formula (I) will exist as a pair of enantiomers (or more than one pair of enantiomers where more than one chiral centre is present in the compound).

The optical isomers may be characterised and identified by their optical activity (i.e. as + and − isomers, or d and l isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastereoisomer). In one general embodiment, 99% or more (e.g. substantially all) of the total amount of the compound of the formula (I) may be present as a single optical isomer (e.g. enantiomer or diastereoisomer).

The compounds of the invention include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

The isotopes may be radioactive or non-radioactive. In one embodiment of the invention, the compounds contain no radioactive isotopes. Such compounds are preferred for therapeutic use. In another embodiment, however, the compound may contain one or more radioisotopes. Compounds containing such radioisotopes may be useful in a diagnostic context.

Esters such as carboxylic acid esters and acyloxy esters of the compounds of formula (I) bearing a carboxylic acid group or a hydroxyl group are also embraced by Formula (I). Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula—C(═O)OR wherein R is:
C₁₋₇alkyl
(e.g., -Me, -Et, -nPr, -iPr, -nBu, -sBu, -iBu, -tBu);
C₁₋₇aminoalkyl (e.g., aminoethyl; 2-(,N-diethylamino)ethyl; 2-(4-morpholino)ethyl); and
acyloxy-C₁₋₇alkyl (e.g., acyloxymethyl;
acyloxyethyl;
pivaloyloxymethyl;
acetoxymethyl;
1-acetoxyethyl;
1-(1-methoxy-l-methyl)ethyl-carbonxyloxyethyl;
1-(benzoyloxy)ethyl; isopropoxy-carbonyloxymethyl;
1-isopropoxy-carbonyloxyethyl; cyclohexyl-carbonyloxymethyl;
1-cyclohexyl-carbonyloxyethyl;
cyclohexyloxy-carbonyloxymethyl;
1-cyclohexyloxy-carbonyloxyethyl;
(4-tetrahydropyranyloxy)carbonyloxymethyl;
1-(4-tetrahydropyranyloxy)carbonyloxyethyl;
(4-tetrahydropyrayl)carbonyloxymethyl; and
1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Biological Activity

The compounds of the formula (I) are inhibitors of cyclin dependent kinases. For example, compounds of the invention have activity against CDK1, CDK2, CDK3, CDK5, CDK6 and CDK7 kinases.

In addition, CDK4, CDK8 and/or CDK9 may be of interest.

Compounds of the invention also have activity against glycogen synthase kinase-3 (GSK-3).

Compounds of the invention also have activity against Aurora kinases.

As a consequence of their activity in modulating or inhibiting CDK and Aurora kinases and glycogen synthase kinase, they are expected to be useful in providing a means of arresting, or recovering control of, the cell cycle in abnormally dividing cells. It is therefore anticipated that the compounds will prove useful in treating or preventing proliferative disorders such as cancers. It is also envisaged that the compounds of the invention will be useful in treating conditions such as viral infections, autoimmune diseases and neurodegenerative diseases for example.

CDKs play a role in the regulation of the cell cycle, apoptosis, transcription, differentiation and CNS function. Therefore, CDK inhibitors could be useful in the treatment of diseases in which there is a disorder of proliferation, apoptosis or differentiation such as cancer. In particular RB+ve tumours may be particularly sensitive to CDK inhibitors. RB-ve tumours may also be sensitive to CDK inhibitors.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermis, liver, lung, for example adenocarcinoma, small cell lung cancer and non-small cell lung carcinomas, oesophagus, gall bladder, ovary, pancreas e.g. exocrine pancreatic carcinoma, stomach, cervix, thyroid, prostate, or skin, for example squamous cell carcinoma; a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma; a hematopoietic tumour of myeloid lineage, for example acute and chronic myelogenous leukemias, myelodysplastic syndrome, or promyelocytic leukemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xenoderoma pigmentoum; keratoctanthoma; thyroid follicular cancer; or Kaposi's sarcoma.

CDKs are also known to play a role in apoptosis, proliferation, differentiation and transcription and therefore CDK inhibitors could also be useful in the treatment of the following diseases other than cancer; viral infections, for example herpes virus, pox virus, Epstein-Barr virus, Sindbis virus, adenovirus, HIV, HPV, HCV and HCMV; prevention of AIDS development in HIV-infected individuals; chronic inflammatory diseases, for example systemic lupus erythematosus, autoimmune mediated glomerulonephritis, rheumatoid arthritis, psoriasis, inflammatory bowel disease, and autoimmune diabetes mellitus; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atropy and cerebellar degeneration; glomerulonephritis; myelodysplastic syndromes, ischemic injury associated myocardial infarctions, stroke and reperfusion injury, arrhythmia, atherosclerosis, toxin-induced or alcohol related lver diseases, haematological diseases, for example, chronic anemia and aplastic anemia; degenerative diseases of the musculoskeletal system, for example, osteoporosis and arthritis, aspirin-senstive rhinosinusitis, cystic fibrosis, multiple sclerosis, kidney diseases and cancer panm.

It has also been discovered that some cyclin-dependent kinase inhibitors can be used in combination with other anticancer agents. For example, the cytotoxic activity of cyclin-dependent kinase inhibitor flavopiridol, has been used with other anticancer agents in combination therapy.

Thus, in the pharmaceutical compositions, uses or methods of this invention for treating a disease or condition comprising abnormal cell growth, the disease or condition comprising abnormal cell growth in one embodiment is a cancer.

Particular subsets of cancers include breast cancer, ovarian cancer, colon cancer, prostate cancer, oesophageal cancer, squamous cancer and non-small cell lung carcinomas.

In the case of compounds having activity against Aurora kinase, particular examples of cancers where it is envisaged that the Aurora kinase inhibiting compounds of the invnention will be useful include:
human breast cancers (e.g. primary breast tumours, node-negative breast cancer, invasive duct adenocarcinomas of the breast, non-endometrioid breast cancers);
ovarian cancers (e.g. primary ovarian tumours);
pancreatic cancers;
human bladder cancers;
colorectal cancers (e.g. primary colorectal cancers);
gastric tumours;
renal cancers;
cervical cancers:
neuroblastomas:
melanomas;
lymphomas;
prostate cancers;
leukemia;
non-endometrioid endometrial carcinomas;
gliomas;
non-Hodgkin's lymphoma;

Cancers which may be particularly amenable to Aurora inhibitors include breast, bladder, colorectal, pancreatic, ovarian, non-Hodgkin's lymphoma, gliomas and nonendometrioid endometrial carcinomas.

In one general embodiment, the cancer may be a cancer other than an oestrogen-receptor mediated cancer, or an oestrogen dependent cancer.

The activity of the compounds of the invention as inhibitors of cyclin dependent kinases, Aurora kinases and glycogen synthase kinase-3 can be measured using the assays set forth in the examples below and the level of activity exhibited by a given compound can be defined in terms of the IC₅₀ value. Preferred compounds of the present invention are compounds having an IC₅₀ value of less than 1 micromole, more preferably less than 0.1 micromole.

Methods for the Preparation of Compounds of the Formula (I)

Compounds of the formula (I) can be prepared in accordance with synthetic methods well known to the skilled person and as described herein.

In this section, unless stated otherwise R¹, R², R³, R⁴ and R⁵ are as herein defined. A synthetic route for the preparation of compounds of the formula (I) in which one of R³ and R⁴ is hydrogen and X is N is illustrated in Scheme 1 below.

As shown in Scheme 1, a substituted or unsubstituted 4-nitro-3-pyrazole carboxylic acid (X¹) is esterified by reaction with thionyl chloride to give the acid chloride intermediate followed by reaction with ethanol to form the ethyl ester (XI). Alternatively, the esterification can be carried out by reacting the alcohol and carboxylic acid in the presence of an acidic catalyst, one example of which is thionyl chloride. The reaction is typically carried out at room temperature using the esterifying alcohol (e.g. ethanol) as the solvent.

The pyrazole 1-nitrogen atom is then protected by means of a suitable protecting group PG, for example an optionally substituted benzyl group such as a para-methoxybenzyl group. The benzyl group (e.g. para-methoxybenzyl group) can be introduced by reacting the nitro ester (XI) with the appropriate benzyl halide in the presence of a base such as sodium carbonate or potassium carbonate. The reaction is typically carried out in a polar solvent such as acetonitrile at ambient temperature.

The N-protected nitro ester (XII) can then be reduced to the corresponding amino compound (XIII) according to standard methods. The reduction may be effected, for example by catalytic hydrogenation in the presence of a catalyst such as palladium on carbon in a polar solvent such as ethanol or dimethylformamide at room temperature.

The amine (AIII) is coupled with an appropriate carboxylic acid R¹—CO₂H under standard amide formation conditions to give the amide (XIV). Thus, for example, the coupling reaction between the carboxylic acid and the amine (XIII) can be carried out in the presence of a reagent of the type commonly used in the formation of peptide linkages. Examples of such reagents include 1,3-dicyclohexylcarbodiimide (DCC) (Sheehan et al, J. Amer. Chem Soc. 1955, 77, 1067), 1-ethyl-3-(3′-dimethylaminopropyl)-carbodiimide (EDC) (Sheehan et al, J. Org. Chem., 1961, 26, 2525), uronium-based coupling agents such as O-(7-azabenzotriazol-1-yl)-N,N,N′,N′-tetramethyluronium hexafluorophosphate (HATU) (L. A. Carpino, J. Amer. Chem. Soc., 1993, 115, 4397) and phosphonium-based coupling agents such as 1-benzo-triazolyloxytris(pyrrolidino)phosphoniua hexafluorophosphate (PyBOP) (Castro et al, Tetrahedron Letters, 1990, 31, 205). Carbodiimide-based coupling agents are advantageously used in combination with 1-hydroxyazobenzotriazole (HOAt) or 1-hydroxybenzotriazole (HOBT) (Konig et al, Chem. Ber., 103, 708, 2024-2034). Preferred coupling reagents include EDC and DCC in combination with HOBt.

The coupling reaction is typically carried out in a non-aqueous, non-protic solvent such as acetonitrile, dioxan, dimethylsulphoxide, dichloromethane, dimethylformamide or N-methylpyrrolidine, or in an aqueous solvent optionally together with one or more miscible co-solvents. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,N-diisopropylethylamine.

As an alternative, a reactive derivative of the carboxylic acid, e.g. an anhydride or acid chloride, may be used. Reaction with a reactive derivative such an anhydride is typically accomplished by stirring the amine and anhydride at room temperature in the presence of a base such as pyridine.

The ester group of the amide (XIV) can then be hydrolysed to give the carboxylic acid (XV) using an alkali metal hydroxide such as lithium hydroxide in a polar aqueous solvent such as a water/tetrahydrofuran mixture, typically at room temperature.

The acid (XV) is reacted with an a-bromoketone (XVI) in the presence of a base such as a carbonate (e.g. caesium carbonate) in an aqueous solvent such as aqueous ethanol, typically with heating to a temperature in the range 50° C. to 100° C., for example approximately 80° C. to give an acyl-methyl ester (XVII) which is then cyclised to the imidazole (XVIII) by heating with ammonium acetate at an elevated temperature (e.g. up to 200° C.) in a high boiling organic solvent such as a xylene (e.g. para-xylene).

As the final step in the synthetic route, the protecting group is removed from the irnidazolyl-pyrazole by standard methods to give a compound of the formula (I) in which R⁴ is hydrogen. For example, where the protecting group is a para-methoxybenzyl group, the protecting group can be removed by treatment with trifluoroacetic acid in the presence of an acid scavenger such as anisole.

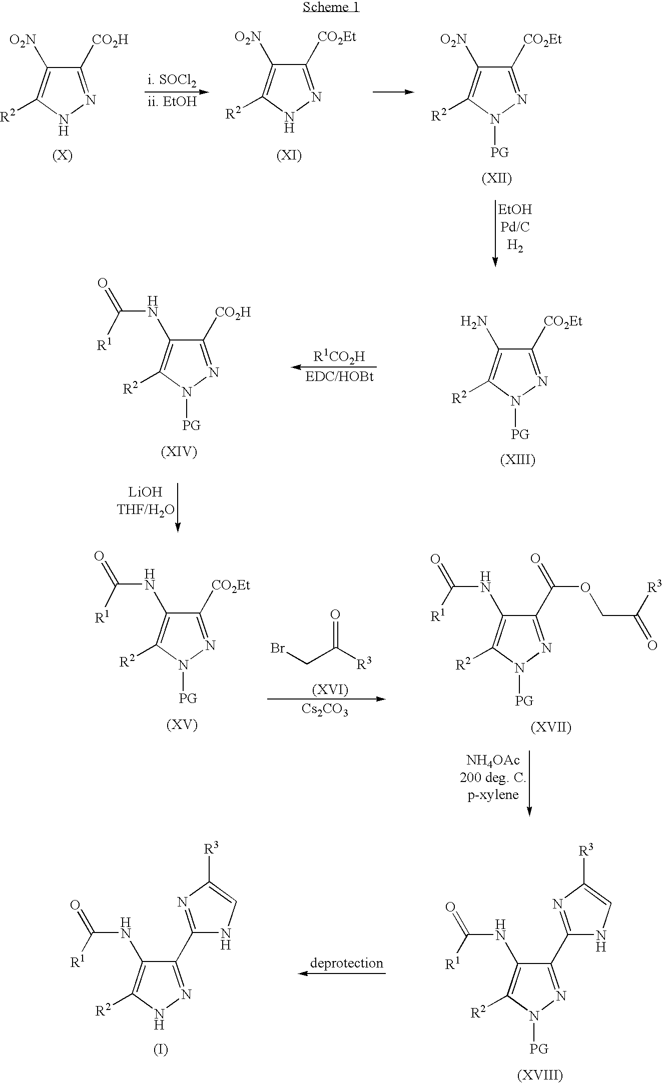

In an alternative route, a 4 amino-pyrazolyl-imidazole unit is constructed and the group R¹-A- is then introduced. Illustrative of this approach are the reaction sequences shown in Schemes 2, 3 and 4, each of which may be used to prepare disubstituted imidazoles.

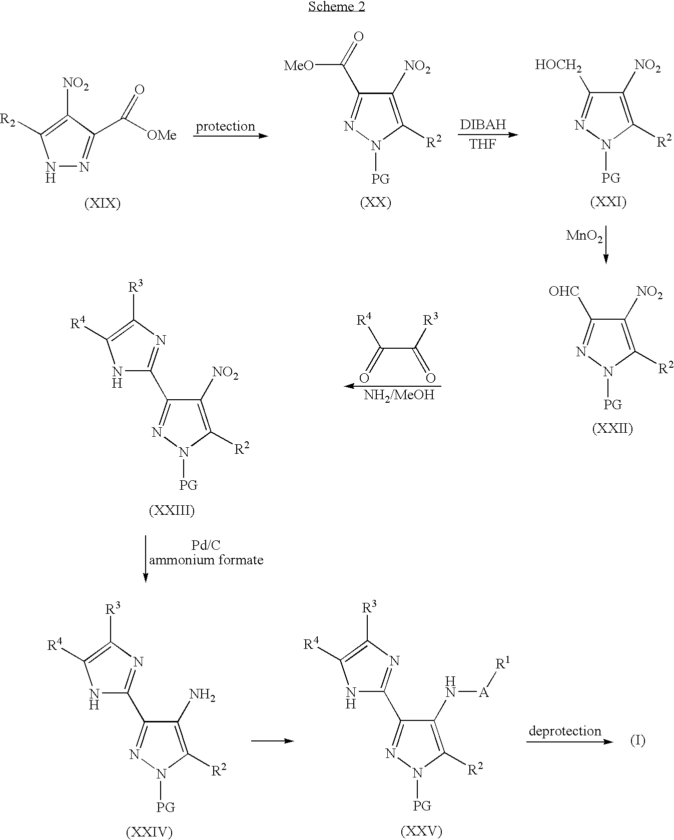

The starting material for the synthetic sequence shown in Scheme 2 is a 4-nitropyrazole methyl ester (XIX) which can be prepared under conditions analogous to those used to prepare the ethyl ester (XI) in Scheme 1. The ester (XIX) is firstly protected by the introduction of a suitable protecting group at the 1-position of the pyrazole ring. One such protecting group is the tetrahydropyranyl (THP) group which can be introduced by reacting the ester (IX) with 3,4-dihydropyran in the presence of an acid such as p-toluene sulphonic acid. The reaction is typically carried out in a solvent such as a chlorinated hydrocarbon, e.g. chloroform, at a temperature from about 0° C. up to about ambient temperature.

The N-protected ester (XX) is reduced to the corresponding alcohol (XXI) using a suitably selective reducing agent such as diisobutyl aluminium hydride (DIBAH) in a polar aprotic solvent such as tetrahydrofuran. The reduction reaction may be carried out at a low temperature, for example from about −78° C. up to ambient temperature. The resulting alcohol (XXI) is then oxidized to the aldehyde (XXII) using an oxidizing agent such as manganese dioxide in a polar solvent such as acetone. The oxidation may be carried out at a mildly elevated temperature, for example a temperature up to the boiling point of the solvent.

The aldehyde (XXII) can be reacted with a 1,2-dione (for example a diaryl 1,2-dione such as benzil or an aryl-alkyl-1,2-dione such as phenyl-propyl-dione) in the presence of ammonia to give the imidazolyl-pyrazole (XXIII). The imidazole ring-forming reaction is typically carried out using an alcoholic solution of ammonia such as methanolic ammonia at room temperature. The nitro-group on the pyrazole ring is then reduced to the corresponding amino group using ammonium formate and palladium on carbon in an aqueous solvent such as aqueous ethanol, typically with heating to a mildly elevated temperature such as 60° C.

The resulting amine (XXIV) can either be firstly de-protected, and then reacted with a reagent suitable for introducing the group R¹-A, or reacted firstly with a reagent suitable for introducing the group R¹-A and then de-protected. The group R¹-A can be introduced for example by reacting the amine with a carboxylic acid R¹—CO₂H or an activated derivative thereof under the amide forming conditions described above. Alternatively, the amine can be reacted with an isocyanate of the formula R¹—N═C═O to give a urea compound of the formula (I) in which A is a group NH(C═O), or with a chloroformate ester of the formula R¹—O—C(═O)—C1 to give a compound of the formula (I) in which A is a group O(C═O).

Deprotection of the protected amine (XIV), or the protected form of a compound of the formula (I), can be achieved using standard methods well known to those skilled in the art. For example, when the protecting group is a TBP group, this car. be removed by warming the compound in a solution containing an acid such as p-toluenesulphonic acid.

The reaction sequence shown in Scheme 2 is of particular use in preparing compounds in which R³ and R⁴ are both substituents.

Scheme 3 illustrates a sequence of reactions that can be used to prepare compounds of the formula (I) in which one or two of R³ and R⁴ are substituents.

The starting point for the sequence of reactions in Scheme 3 is the nitro-pyrazole carboxylic acid (X) which is reacted with an amine of the formula R⁴COCH₂NH₂ under standard amide forming conditions of the type described above to give amide (XXVI). The amide is then protected by the introduction of a protecting group at the pyrazole 1-position:—this can be achieved using the TRP protecting group under conditions analogous to those described above in relation to Scheme 2.

At this stage, the protected nitro-pyrazole compound (XXVII) may be cyclised by reacting with ammonium acetate in acetic acid with heating to give to an imidazole of the formula (XXIX) wherein R³ is hydrogen. Alternatively, the protected nitro-pyrazole compound (XXVII) can be reacted with a compound R³-L where L is a leaving group or atom such as a halogen (e.g. bromine) in the presence of a strong base such as a metal hydride (e.g. sodium hydride) to give a compound of the formula (XXVIII) in which R³ is a substituent such as an alkyl, aralkyl, heteroarylalkyl or allyl group.

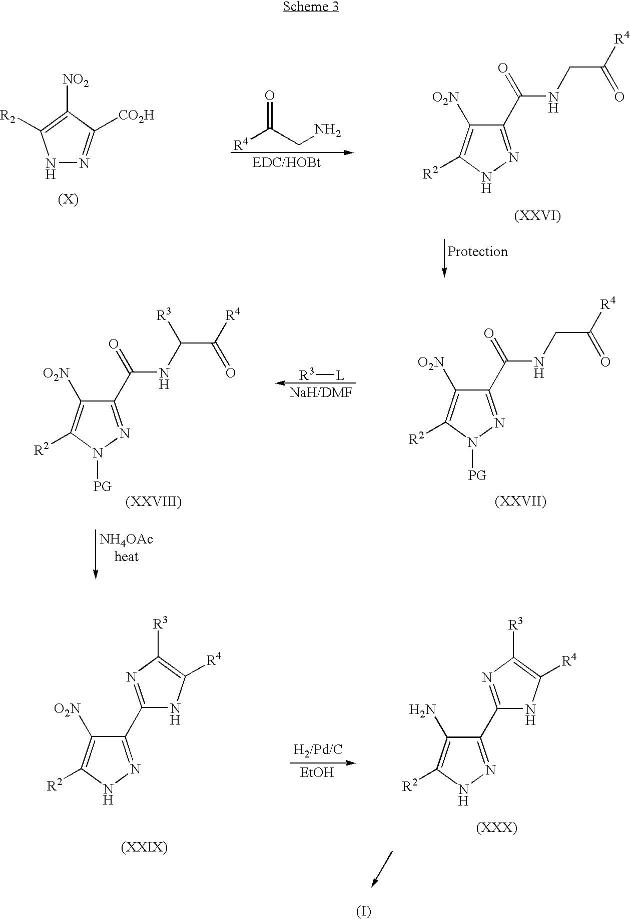

The reaction with the compound of formula R³-L is typically carried out in a non-protic polar solvent such as dimethylformamide (DMF), usually at a reduced temperature, e.g. a temperature less than 0° C.

The compound of the formula (XXVIII) may then be subjected to cyclisation by reaction with ammonium acetate in the presence of acetic acid. The cyclisation reaction is typically carried out with heating, for example to a temperature in excess of 100° C. (e.g. up to about 120° C.), a preferred source of heat being a microwave heater. By appropriate selection of the reaction conditions, the protecting group PG may be removed during the cyclisation step. Alternatively, if the protecting group survives the cyclisation step, it can subsequently be removed by standard methods.

The resulting nitro-pyrazolyl-imidazole compound (XXIX) can be reduced to the corresponding amine (XX) by standard methods, for example using hydrogen in the presence of a palladium on carbon catalyst. The amine (XXX) can be converted into a compound of the formula (I) by treatment with a carboxylic acid, or activated derivative thereof, or with an isocyanate or chloroformate ester according to the methods described above.

A further synthetic route to the compounds of the formula (I) is illustrated in Scheme 4.

In Scheme 4, the starting point is the N-protected pyrazolyl carboxylic acid (XV) (see Scheme 1) which can be reacted with amine (XXXI) under standard amide forming conditions to form amide (XXXII). At this stage, a substituent group R³ can be introduced by reaction with a compound R³-L (where L is a leaving group or atom such as bromine) in the presence of a base such as sodium hydride under conditions analogous to those described above in relation to Scheme 3 to give a compound of the formula (XXXIII). The amides (XXXII) and (XXXIII) can each be cyclised to form a compound of the formula (I) in which A is CO and R³ is hydrogen or a substituent group respectively, cyclisation being achieved by reaction with ammonium acetate in acetic acid at an elevated temperature (e.g. up to about 150°).

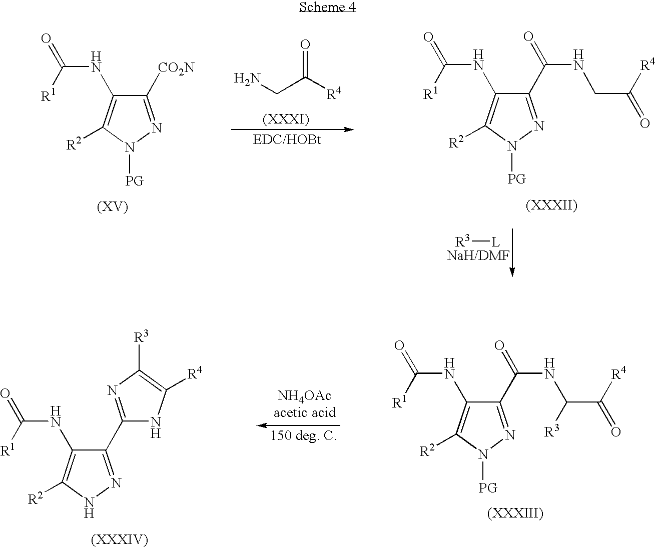

A variation on the synthetic routes shown in Schemes 1 and 2 is illustrated in Scheme 5. In Scheme 5, the N-protected pyrazole carboxylic acid ester (XIV) (see Scheme 1) is firstly treated with a reducing agent such as diisobutyl aluminium hydride (DIBAL) to reduce the carboxylic acid ester to an alcohol group CH₂OH (not shown) which is then oxidised to give an aldehyde (XXXIV) using an oxidising agent such as manganese dioxide. The hydride reduction with DIBAL is conveniently carried out in a polar aprotic solvent such as THF, usually at a temperature below ambient temperature, for example at −78° C. The oxidation step can be carried out at ambient temperature in a polar solvent such as acetone.

The 3-formyl pyrazole (XXXIV) is then cyclised to form an imidazole ring by reaction with ammonium acetate and a compound of the R³C(O)C(O)R⁴ at an elevated temperature, for example a temperature between 80° C. and 120° C. The protecting group PG on the resulting compound (XVIII) is then removed by standard procedures as described above to give the compound of the formula (I).

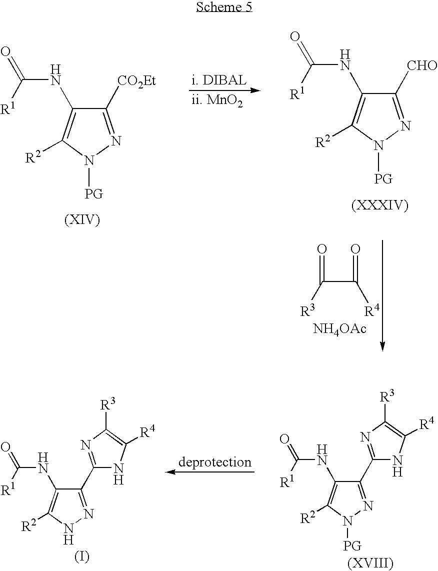

In a variation of the synthetic route shown in Scheme 5, the 3-formyl pyrazole compound (XXXIV) is reacted with a substituted dinethoxydione compound of the formula (XXXV) in the presence of ammonia to form a a 4-formyl substituted imidazole (XXXVI) or its diethyl acetal (not shown). Where the diethylacetal compound is formed, this can be decomposed to give the 4-formyl compound (XXXVI) by treatment with an acid such as p-toluenesulphonic acid. The cyclisation reaction with ammonia can be carried out at room temperature in a polar solvent such as an alcohol, e.g. methanol.

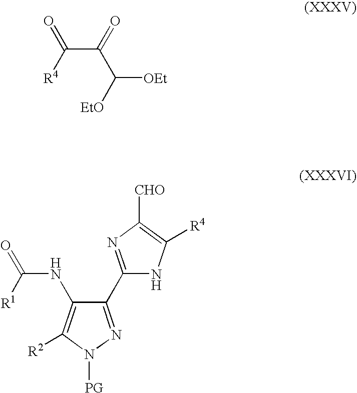

The 4-formyl compound (ANTI) can then be converted into a range of compounds of the formula (I). For example, it can be reduced to give a compound wherein R³ is hydroxymethyl, or it can be reductively aminated using an amine such as morpholine or methoxyethylamine in the presence of sodium triacetoxy borohydride.

In a further variation on the synthetic route shown in Scheme 5, an oxime R⁴—C(O)—C(NOH)—R³ can be used in place of the dione R³C(O)C(O)R⁴. The oxime is reacted with the 3-formyl pyrazole (XXIV) to give the N-hydroxy imidazole compound (XXXVII):

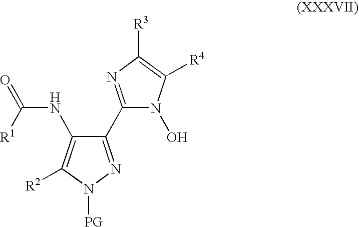

The N-hydroxy group can then be removed using TiCl₃ in aqueous acidic methanol at a reduced temperature, for example at around 0° C. to five the compound of the formula (I).

A ftrther synthetic route to compounds of the formula (I) is shown in Scheme 6 below.

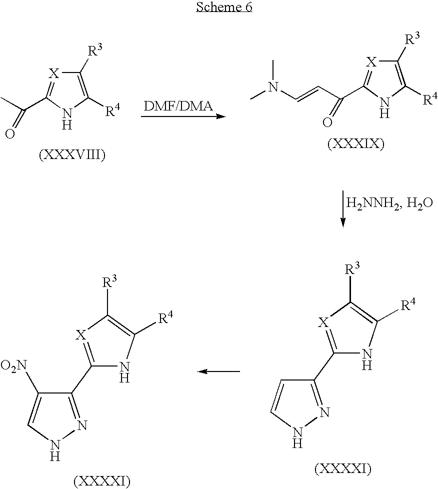

As illustrated in Scheme 3, the ketone (XXXVIII) can be reacted with dimethylformamide-dimethylacetal at elevated temperature gives an α,β-unsaturated ketone (XXXIX) (Jachak et al, Montash. Chem., 1993,124(2), 199-207), which upon heating with hydrazine hydrate gives a pyrazole of formula (XXX). This can then be nitrated as discussed herein to give the nitropyrazole

The procedure illustrated in Scheme 3 is of particular utility in the preparation of compounds when X is a group CR⁵.

Compounds of the formula (I) in which A is NH(CO) can be prepared using standard methods for the synthesis of ureas. For example, such compounds can be prepared by reacting an aminopyrazole compound of the formula (XX) with a suitably substituted phenylisocyanate in a polar solvent such as DMF. The reaction is conveniently carried out at room temperature.

Compounds of the formula (I) in which A is bond can be prepared using the Schemes described previously using standard methods for the synthesis of secondary and tertiary amines. For example, such compounds can be prepared by reacting an aminopyrazole compound of the formula (XXX) with a suitably substituted alkylating agent in a polar solvent such as DMF. The reaction is conveniently carried out at room temperature.

The starting materials for the synthetic routes shown in the Schemes above, the pyrazoles of Formula (X), can either be obtained commercially or can be prepared by methods known to those skilled in the art. They can be obtained using known methods e.g. from ketones, such as in a process described in EP308020 (Merck), or the methods discussed by Schmidt in Helv. Chim. Acta., 1956, 39, 986-991 and Helv. Chim. Acta., 1958, 41, 306-309. Alternatively they can be obtained by conversion of a commercially available pyrazole, for example those containing halogen, nitro, ester, or amide functionalities, to pyrazoles containing the desired functionality by standard methods known to a person skilled in the art. For example, in 3-carboxy-4-nitropyrazole, the nitro group can be reduced to an amine by standard methods. 4-Nitro-pyrazole-3-carboxylic acid (X) can either be obtained commercially or can be prepared by nitration of the corresponding 4-unsubstituted pyrazole carboxy compound, and pyrazoles containing a halogen, may be utilized in coupling reactions with tin or palladium chemistry. A substituted or unsubstituted 4-nitro-3-pyrazole carboxylic acid can be esterified by reaction with thionyl chloride to give the acid chloride intermediate followed by reaction with an alcohol to form the ester of formula (XI). Alternatively, the esterification can be carried out by reacting the alcohol and carboxylic acid in the presence of an acidic catalyst, one example of which is thionyl chloride. The reaction is typically carried out at room temperature using the esterifying alcohol (e.g. ethanol) as the solvent.

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999). A hydroxy group may be protected, for example, as an ether (—OR) or an ester (—OC(═O)R), for example, as: a t-butyl ether; a benzyl, benzhydryl (diphenylmethyl), or trityl (triphenylmethyl) ether; a trimethLylsilyl or t-butyldimethiylsilyl ether; or an acetyl ester (—OC(═O)CH₃, —OAc). An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. An amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH—Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH—Bpoc), as a 9-fluorenyimethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2(-phenylsulphonyl)ethyloxy amide (—NH-Psec). Other protecting groups for amines, such as cyclic amines and heterocyclic N-H groups, include toluenesulfonyl (tosyl) and methanesulfonyl (mesyl) groups and benzyl groups such as a para-methoxybenzyl (PMB) group, or a tetrahydropyranyl (THP) group. A carboxylic acid group may be protected as an ester for example, as: an C₁₋₇ alkyl ester (e.g., a methyl ester; a t-butyl ester); a C₁₋₇ haloalkyl ester (e.g., a C₁₋₇ tnhaloalkyl ester); a triC₁₋₇ alkylsilyl-C₁₋₇alkyl ester; or a C₅₋₂₀ aryl-C₁₋₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(═O)CH₃).

Methods of Purification

The compounds may be isolated and purified by a number of methods well known to those skilled in the art and examples of such methods include chromatographic techniques such as column chromatography (e.g. flash chromatography) and HPLC. Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

One such system for purifying compounds via preparative LC-MS is described in the experimental section below although a person skilled in the art will appreciate that alternative systems and methods to those described could be used. In particular, normal phase preparative LC based methods might be used in place of the reverse phase methods described here. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc as outlined in the analytical methods described above could alternatively be used to purify the compounds.

Pharmaceutical Formulations

While it is possible for the active compound to be administered alone, it is preferable to present it as a pharmaceutical composition (e.g. formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defmed above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions containing compounds of the formula (I) can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and grarnlating agents such as polyiinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastrointestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a furher alternative, the active compound can be formulated in a delivery system that provides osmotic control of the release of the compound. Osmotic release and other delayed release or sustained release formulations may be prepared in accordance with methods well known to those skilled in the art.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the inventions will generally be presented in unit dosage form and, as such, will typically contain sufficient compound to provide a desired level of biological activity. For example, a formulation intended for oral administration may contain from 0.1 milligrams to 2 grams of active ingredient, more usually from 10 milligrams to 1 gram, for example, 50 milligrams to 500 milligrams. The active compound will be administered to a patient in need thereof (for example a human or animal patient) in an amount sufficient to achieve the desired therapeutic effect.

Methods of Diagnosis and Treatment

It is envisaged that the compounds of the formula (I) will useful in the prophylaxis or treatment of a range of disease states or conditions mediated by cyclin dependent kinases, glycogen synthase kinase-3 and Aurora kinases. Examples of such disease states and conditions are set out above.

Compounds of the formula (I) are generally administered to a subject in need of such administration, for example a human or animal patient, preferably a human. The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity. The compounds may be administered over a prolonged term to maintain beneficial therapeutic effects or may be administered for a short period only. Alternatively they may be administered in a pulsatile or continuous manner.

A typical daily dose of the compound can be in the range from 100 picograms to 100 milligrams per kilogram of body weight, more typically 10 nanograms to 10 milligrams per kilogram of bodyweight although higher or lower doses may be administered where required. Ultimately, the quantity of compound administered and the type of composition used will be commensurate with the nature of the disease or physiological condition being treated and will be at the discretion of the physician.

The compounds of the formula (I) can be administered as the sole therapeutic agent or they can be administered in combination therapy with one of more other compounds for treatment of a particular disease state, for example a neoplastic disease such as a cancer as hereinbefore defined. Examples of other therapeutic agents that may be administered together (whether concurrently or at different time intervals) with the compounds of the formula (I) include but are not limited to topoisomerase inhibitors, alkylating agents, antimetabolites, DNA binders and microtubule inhibitors (tubulin targeting agents), such as cisplatin, cyclophosphamide, doxorubicin, irinotecan, fludarabine, 5FU, taxanes, mitomycin C, or radiotherapy. For the case of CDK or Aurora inhibitors combined with other therapies, the two or more treatments may be given in individually varying dose schedules and via different routes.

Where the compound of the formula (I) is administered in combination therapy with one, two, three, four or more other therapeutic agents (preferably one or two, preferably one), the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

For use in combination therapy with another chemotherapeutic agent, the compound of the formula (I) and one, two, three, four or more other therapeutic agents can be, for example, formulated together in a dosage form containing two, three, four or more therapeutic agents. In an alternative, the individual therapeutic agents may be formulated separately and presented together in the form of a kit, optionally with instructions for their use.

A person skilled in the art would know through their common general knowledge the dosing regimes and combination therapies to use.

Prior to administration of a compound of the formula (I), a patient may be screened to determine whether a disease or condition from which the patient is or may be suffering is one which would be susceptible to treatment with a compound having activity against Aurora kinases. For example, a biological sample taken from a patient may be analysed to determine whether a condition or disease, such as cancer, that the patient is or may be suffering from is one which is characterised by upregulation of Aurora kinase, this includes elevated expression or over-expression of Aurora kinase, including gene amplification (i.e. multiple gene copies) and increased expression by a transcriptional effect, and hyperactivity and activation of Aurora kinase, including activation by mutations. Thus, the patient may be subjected to a diagnostic test to detect a marker characteristic of over-expression, up-regulation or activation of Aurora kinase. The term diagnosis includes screening. By marker we include genetic markers including, for example, the measurement of DNA composition to identify mutations of Aurora or CDC4. The term marker also includes markers which are characteristic of up regulation of Aurora or cyclin E, including enzyme activity, enzyme levels, enzyme state (e.g. phosphorylated or not) and MRNA levels of the aforementioned proteins.

The diagnostic tests are typically conducted on a biological sample selected from tumour biopsy samples, blood samples (isolation and enrichment of shed tumour cells), stool biopsies, sputum, chromosome analysis, pleural fluid, peritoneal fluid, or urne.

It has been found, see Ewart-Toland et al., (Nat Genet. 2003 August;34(4):403-12), that individuals forming part of the sub-population possessing the Ile31 variant of the STK gene (the gene for Aurora kinase A) may have an increased susceptibility to certain forms of cancer. It is envisaged therefore that such individuals suffering from cancer will benefit from the administration of compounds having Aurora kinase inhibiting activity. A patient suffering from, or suspected of suffering from, a cancer may therefore be screened to determine whether he or she forms part of the Ile31 variant sub-population. The screening process will typically involve direct sequencing, oligonucleotide microarray analysis, or a mutant specific antibody.

Tumours with activating mutants of Aurora or up-regulation of Aurora including any of the isoforms thereof, may be particularly sensitive to Aurora inhibitors. Tumours may preferentially be screened for up-regulation of Aurora or for Aurora possessing the Ile31 variant prior to treatment (Ewart-Toland et al., Nat Genet. 2003 August; 34(4):403-12). Ewart-Toland et al identified a common genetic variant in STK15 (resulting in the amino acid substitution F31I) that is preferentially amplified and associated with the degree of aneuploidy in human colon tumors. These results are consistent with an important role for the Ile3 1 variant of STK15 in human cancer susceptibility.

The aurora A gene maps to the chromosome 20q13 region that is frequently amplified in many cancers e.g. breast, bladder, colon, ovarian, pancreatic. Patients with a tumour that has this gene amplification might be particularly sensitive to reatments targeting aurora kinase inhibition

Methods of identification and analysis of Aurora mutations and up-regulation of Aurora isoforms and chromosome 20q13 amplification are known to a person skilled in the art. Screening methods could include, but are not limited to, standard methods such as reverse-transcriptase polymerase chain reaction (RT-PCR) or in-situ hybridisation.

In screening by RT-PCR, the level of Aurora mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd}Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing Aurora mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtiter plates, Western blotting, 2-dimensional SDS-polyacrylamide gel electrophoresis, ELISA, flow cytometry and other methods known in the art for detection of specific proteins. Detection methods would include the use of site specific antibodies. The skilled artisan will recognize that all such well-known techniques for detection of Aurora up-regulation and mutants of Aurora could be applicable in the present case.

In addition, all of these techniques could also be used to identify tumours particularly suitable for treatment with CDK inhibitors. Tumours with mutants of CDC4 or up-regulation, in particular over-expression, of cyclin E or loss of p21 or p27 may be particularly sensitive to CDK inhibitors. Tumours may preferentially be screened for up-regulation, in particular over-expression, of cyclin E (Harwell R M, Mull B B, Porter D C, Keyornarsi K.; J Biol Chem. 2004 Mar. 26; 279(13):12695-705) or loss of p21 or p27 or for CDC4 variants prior to treatment (Rajagopalan H, Jallepalli P V, Rago C, Velculescu V E, Kinzler K W, Vogelstein B, Lengauer C.; Nature. 2004 Mar. 4; 428(6978):77-81).

Antifungal Use

In a further aspect, the invention provides the use of the compounds of the formula (I) as hereinbefore defined as antifungal agents.

The compounds of the formula (I) may be used in animal medicine (for example in the treatment of mammals such as humans), or in the treatment of plants (e.g. in agriculture and horticulture), or as general antifingal agents, for example as preservatives and disinfectants.

In one embodiment, the invention provides a compound of the formula (I) as hereinbefore defined for use in the prophylaxis or treatment of a fingal infection in a mammal such as a human.

Also provided is the use of a compound of the formula (I) for the manufacture of a medicament for use in the prophylaxis or treatment of a fungal infection in a mammal such as a human.

For example, compounds of the invention may be administered to human patients suffering from, or at risk of infection by, topical fungal infections caused by among other organisms, species of Candida, Trichophyton, Microsporum or Epidermophyton, or in mucosal infections caused by Candida albicans (e.g. thrush and vaginal candidiasis). The compounds of the invention can also be administered for the treatment or prophylaxis of systemic fungal infections caused by, for example, Candida albicans, Cryptococcus neoformans, Aspergillus flavus, Aspergillus fumigatus, Coccidiodies, Paracoccidioides, Histoplasma or Blastomyces.

In another aspect, the invention provides an antifungal composition for agricultural (including horticultural) use, comprising a compound of the formula (I) together with an agriculturally acceptable diluen.t or carier.

The invention further provides a method of treating an animal (including a mammal such as a human), plant or seed having a fingal infection, which comprises treating said animal, plant or seed, or the locus of said plant or seed, with an effective amount of a compound of the formula (I).

The invention also provides a method of treating a fungal infection in a plant or seed which comprises treating the plant or seed with an antifungally effective amount of a fungicidal composition containing a compound of the formula (I) as hereinbefore defined.

Differential screening assays may be used to select for those compounds of the present invention with specificity for non-human CDK enzymes. Compounds which act specifically on the CDK enzymes of eukaryotic pathogens can be used as anti-fungal or anti-parasitic agents. Inhibitors of the Candida CDK kinase, CKSI, can be used in the treatment of candidiasis. Antifungal agents can be used against infections of the type hereinbefore defined, or opportunistic infections that commonly occur in debilitated and immunosuppressed patients such as patients with leukemias and lymphomas, people who are receiving immunosuppressive therapy, and patients with predisposing conditions such as diabetes mellitus or AIDS, as well as for non-immunosuppressed patients.

Assays described in the art can be used to screen for agents which may be useful for inhibiting at least one fungus implicated in mycosis such as candidiasis, aspergillosis, mucormycosis, blastomycosis, geotrichosis, cryptococcosis, chromoblastomycosis, coccidiodomycosis, conidiosporosis, histoplasmosis, maduromycosis, rhinosporidosis, nocaidiosis, para-actinomycosis, penicilliosis, monoliasis, or sporotrichosis. The differential screening assays can be used to identify anti-fungal agents which may have therapeutic value in the treatment of aspergillosis by making use of the CDK genes cloned from yeast such as Aspergillus fumigatus, Aspergillus flavus, Aspergillus niger, Aspergillus nidulans, or Aspergillus terreus, or where the mycotic infection is mucon-nycosis, the CDK assay can be derived from yeast such as Rhizopus arrilzias, Rhizopus oryzae, Absidia corymbifera, Absidia ramosa, or Mucorpusillus. Sources of other CDK enzymes include the pathogen Pneumocystis carinii.

By way of example, in vitro evaluation of the antifungal activity of the compounds can be performed by determining the minimum inhibitory concentration (M.I.C.) which is the lowest concentration of the test compounds, in a suitable medium, at which growth of the particular microorganism fails to occur. In practice, a series of agar plates, each having the test compound incorporated at a particular concentration is inoculated with a standard culture of, for example, Candida albicans and each plate is then incubated for an appropriate period at 37° C. The plates are then examined for the presence or absence of growth of the fimgus and the appropriate M.I.C. value is noted. Alternatively, a turbidity assay in liquid cultures can be performed and a protocol outlining an example of this assay can be found in Example 51.

The in vivo evaluation of the compounds can be carried out at a series of dose levels by intraperitoneal or intravenous injection or by oral administration, to mice that have been inoculated with a fungus, e.g., a strain of Candida albicans or Aspergillus flavus. The activity of the compounds can be assessed by monitoring the growth of the fungal infection in groups of treated and untreated mice (by histology or by retrieving fungi from the infection). The activity may be measured in terms of the dose level at which the compound provides 50% protection against the lethal effect of the infection (PD₅₀).

For human antifungal use, the compounds of the formula (I) can be administered alone or in admixture with a pharmaceutical carrier selected in accordance with the intended route of administration and standard pharmaceutical practice. Thus, for example, they may be administered orally, parenterally, intravenously, intramuscularly or subcutaneously by means of the formulations described above in the section headed “Pharmaceutical Formulations”.

For oral and parenteral administration to human patients, the daily dosage level of the antifungal compounds of the formula (I) can be from 0.01 to 10 mg/kg (in divided doses), depending on inter alia the potency of the compounds when administered by either the oral or parenteral route. Tablets or capsules of the compounds may contain, for example, from 5 mg to 0.5 g of active compound for administration singly or two or more at a time as appropriate. The physician in any event will determine the actual dosage (effective amount) which will be most suitable for an individual patient and it will vary with the age, weight and response of the particular patient.

Alternatively, the antifungal compounds of formula (I) can be administered in the form of a suppository or pessary, or they may be applied topically in the form of a lotion, solution, cream, ointment or dusting powder. For example, they can be incorporated into a cream consisting of an aqueous emulsion of polyethylene glycols or liquid paraffin; or they can be incorporated, at a concentration between 1 and 10%, into an ointment consisting of a white wax or white soft paraffin base together with such stabilizers and preservatives as may be required.

In addition to the therapeutic uses described above, anti-fungal agents developed with such differential screening assays can be used, for example, as preservatives in foodstuff, feed supplement for promoting weight gain in livestock, or in disinfectant formulations for treatment of non-living matter, e.g., for decontaminating hospital equipment and rooms. In similar fashion, side by side comparison of inhibition of a mammalian CDK and an insect CDK, such as the Drosophilia CDK5 gene (Hellmich et al. (1994) FEBS Lett 356:317-21), will permit selection amongst the compounds herein of inhibitors which discriminate between the human/mammalian and insect enzymes. Accordingly, the present invention expressly contemplates the use and formulation of the compounds of the invention in insecticides, such as for use in management of insects like the fruit fly.

In yet another embodiment, certain of the subject CDK inhibitors can be selected on the basis of inhibitory specificity for plant CDK's relative to the mammalian enzyme. For example, a plant CDK can be disposed in a differential screen with one or more of the human enzymes to select those compounds of greatest selectivity for inhibiting the plant enzyme. Thus, the present invention specifically contemplates foimulations of the subject CDK inhibitors for agricultural applications, such as in the form of a defoliant or the like.

For agricultural and horticultural purposes the compounds of the invention may be used in the form of a composition formulated as appropriate to the particular use and intended purpose. Thus the compounds may be applied in the form of dusting powders, or granules, seed dressings, aqueous solutions, dispersions or emulsions, dips, sprays, aerosols or smokes. Compositions may also be supplied in the form of dispersible powders, granules or grains, or concentrates for dilution prior to use. Such compositions may contain such conventional carriers, diluents or adjuvants as are known and acceptable in agriculture and horticulture and they can be manufactured in accordance with conventional procedures. The compositions may also incorporate other active ingredients, for example, compounds having herbicidal or insecticidal activity or a further fungicide. The compounds and compositions can be applied in a number of ways, for example they can be applied directly to the plant foliage, stems, branches, seeds or roots or to the soil or other growing medium, and they may be used not only to eradicate disease, but also prophylactically to protect the plants or seeds from attack. By way of example, the compositions may contain from 0.01 to 1 wt. % of the active ingredient. For field use, likely application rates of the active ingredient may be from 50 to 5000 g/hectare.

The invention also contemplates the use of the compounds of the formula (I) in the control of wood decaying fungi and in the treatment of soil where plants grow, paddy fields for seedlings, or water for perfusion. Also contemplated by the invention is the use of the compounds of the formula (I) to protect stored grain and other non-plant loci from fingal infestation.

### EXAMPLES

The invention will now be illustrated, but not limited, by reference to the specific embodiments described in the following examples.

In the examples, the compounds prepared were characterised by liquid chromatography and mass spectroscopy using the systems and operating conditions set out below. Where chlorine is present, the mass quoted for the compound is for ³⁵C1. Several systems were used, as described below, and these were equipped with were set up to run under closely similar operating conditions. The operating conditions used are also described below.

Platform System 1

System: Waters 2790/Platform LC

Mass Spec Detector: Micromass Platform LC

PDA Detector: Waters 996 PDA

Analytical Conditions:

Eluent A: 5% CH₃CN in 95% H₂O (0.1% Formic Acid)

Eluent B: CH₃CN (0.1% Formic Acid)

Gradient: 10-95% eluent B

Flow: 1.2 ml/min

Column: Synergi 4 μm Max-RP C₁₂, 80A, 50×4.6 mm (Phenomenex)

MS Conditions:

Capillary voltage: 3.5 kV

Cone voltage: 30 V

Source Temperature: 120° C.

FractionLynx System 1

System: Waters FractionLynx (dual analytical/prep)

Mass Spec Detector: Waters-Micromass ZQ

PDA Detector: Waters 2996 PDA

Analytical Conditions:

Eluent A: H₂O(0.1% Formic Acid)

Eluent B: CH₃CN (0.1% Formic Acid)

Gradient: 5-95% eluent B

Flow: 1.5 ml/min

Column: Synergi 4 μm Max-RP C₁₂, 80A, 50×4.6 mm (Phenomenex)

MS Conditions:

Capillary voltage: 3.5 kV

Cone voltage: 30 V

Source Temperature: 120° C.

Desolvation Temperature: 300° C.

Platform System 2

HPLC System: Waters 2795

Mass Spec Detector: Micromass Platform LC

PDA Detector: Waters 2996 PDA

Acidic Analytical Conditions:

Eluent A: H₂O (0.1% Formic Acid)

Eluent B: CH₃CN (0.1% Formic Acid)

Gradient: 5-95% eluent B over 3.5 minutes

Flow: 1.5 ml/min

Column: Phenomenex Synergi 4μ Max-RP 80A, 50×4.6 mm

Basic Analvtical Conditions:

Eluent A: H₂O (10 mM NH₄HCO₃ buffer adjusted to pH=9.5 with NH₄OH)

Eluent B: CH₃CN

Gradient: 05-95% eluent B over 3.5 minutes

Flow: 1.5 ml/min

Column: Waters XTerra MS C₁₈ 5μ 4.6×50 mm

Polar Analytical Conditions:

Eluent A: H₂O (0.1% Formic Acid)

Fluent B: CH₃CN (0.1% Formic Acid)

Gradient: 00-50% eluent B over 3 minutes

Flow: 1.5 ml/min

Column: Phenomenex Synergi 4μ Hydro 80A, 50×4.6 mm

MS Conditions:

Capillary voltage: 3.5 kV

Cone voltage: 30 V

Source Temperature: 120° C.

Scan Range: 165-700 amu

Ionisation Mode: ElectroSpray Negative, Positive or Positive & Negative

FractionLynx System 2

System: Waters FractionLynx (dual analytical/prep)

HPLC Pump: Waters 2525

Injector-Autosampler: Waters 2767

Mass Spec Detector: Waters-Micromass ZQ

PDA Detector: Waters 2996 PDA

Analytical Conditions:

Eluent A: H₂O (0.1% Formic Acid)

Eluent B: CH₃CN (0.1% Formic Acid)

Gradient: 5-95% eluent B over 5 minutes

Flow: 2.0 ml/min

Column: Phenomenex Synergi 4μ Max-RP 80A, 50×4.6 mm

Polar Analvtical Conditions:

Eluent A: H₂O (0.1% Formic Acid)

Eluent B: CH₃CN (0.1% Formic Acid)

Gradient: 00-50% eluent B over 5 minutes

Flow: 2.0 ml/min

Column: Phenomenex Synergi 4μ Max-RP 80A, 50×4.6 mm

MS conditions:

Capillary voltage: 3.5 kV

Cone voltage: 25 V

Source Temperature: 120° C.

Scan Range: 125-800 amu

Ionisation Mode: ElectroSpray Positive or ElectroSpray Positive & Negative

Mass Directed Purification LC-MS System

The following preparative chromatography systems can be used to purify the compounds of the invention.

Hardware:

Waters Fractionlynx system:

2767 Dual Autosampler/Fraction Collector

2525 preparative pump

CFO (column fluidic organiser) for column selection

RMA (Waters reagent manager) as make up pump

Waters ZQ Mass Spectrometer

Waters 2996 Photo Diode Array detector

Software: Masslynx 4.0

Columns:

1. Low pH chromatography: Phenomenex Synergy MAX-RP, 10l, 150×15 mm (alternatively used same column type with 100×21.2 mm dimensions).

2. High pH chromatography: Phenomenex Luna C18 (2), 10μ, 100×21.2 mm (alternatively used Thermo Hypersil Keystone BetaBasic C18, 5μ, 100×21.2 mm)

Eluents:

1. Low pH chromatography:

Solvent A: H₂O+0.1% Formic Acid, pH 1.5

Solvent B: CH₃CN+0.1% Formic Acid

2. High pH chromatography:

Solvent A: H₂O+10 mM NH₄HCO₃+NH₄OH, pH 9.5

Solvent B: CH₃CN

3. Make up solvent: MeOH+0.1% formic acid (for both chromatography type)

Methods:

Prior to using preparative chromatography to isolate and purify the product compounds, analytical LC-MS can first be used to determine the most appropriate conditions for preparative chromatography. A typical routine is to run an analytical LC-MS using the type of chromatography (low or high pH) most suited for compound structure. Once the analytical trace shows good chromatography, a suitable preparative method of the same type can be chosen. Typical running condition for both low and high pH chromatography methods are:

Flow rate: 24 ml/min

Gradient: Generally all gradients have an initial 0.4 min step with 95% A+5% B. Then according to analytical trace a 3.6 min gradient is chosen in order to achieve good separation (e.g. from 5% to 50% B for early retaining compounds; from 35% to 80% B for middle retaining compounds and so on)

Wash: 1 minute wash step is performed at the end of the gradient

Re-equilibration: A 2.1 minute re-equilibration step is carried out to prepare the system for the next run

Make Up flow rate: 1 ml/min

Solvent:

All compounds were usually dissolved in 100% MeOH or 100% DMSO

MS running Conditions:

Capillary voltage: 3.2 kV

Cone voltage: 25 V

Source Temperature: 120° C.

Multiplier: 500 V

Scan Range: 125-800 amu

Ionisation Mode: ElectroSpray Positive

Analytical LC-MS System

HPLC System: Waters 2795

Mass Spec Detector: Micromass Platform LC

PDA Detector: Waters 2996 PDA

Acidic Analytical Conditions:

Eluent A: H₂O (0.1% Formic Acid)

Eluent B: CH₃CN (0.1% Formic Acid)

Gradient: 5-95% eluent B over 3.5 minutes

Flow: 0.8 ml/min

Column: Phenomenex Synergi 4μ MAX-RP 80A, 2.0×50 mm

Basic Analytical Conditions:

Eluent A: H₂O (10 mM NH₄HCO₃ buffer adjusted to pH═9.5 with NH₄OH)

Eluent B: CH₃CN

Gradient: 05-95% eluent B over 3.5 minutes

Flow: 0.8 ml/min

Column: Thermo Hypersil-Keystone BetaBasic-18 5 μm 2.1×50 mm or

Column: Phenomenex Luna C18(2) 5 μm 2.0×50 mm

Polar Analytical Conditions:

Eluent A: H₂O (0.1% Formic Acid)

Eluent B: CH₃CN (0.1% Formic Acid)

Gradient: 00-50% eluent B over 3 minutes

Flow: 0.8 ml/min

Column: Thermo Hypersil-Keystone HyPurity Aquastar, 5μ, 2.1×50 mm or

Column: Phenomenex Synergi 4μ MAX-RP 80A, 2.0×50 mm or

Longer Analytical Conditions:

Eluent A: H₂O (0.1% Formic Acid)

Eluent B: CH₃CN (0.1% Formic Acid)

Gradient: 05-95% eluent B over 15 minutes

Flow: 0.4 m/min

Column: Phenomenex Synergi 4μ MAX-RP 80A, 2.0×150 mm

MS Conditions:

Capillary voltage: 3.6 kV

Cone voltage: 30 V

Source Temperature: 120° C.

Scan Range: 165-700 amu

Ionisation Mode: ElectroSpray Positive or
ElectroSpray Negative or
ElectroSpray Positive & Negative

The starting materials for each of the Examples are commercially available unless otherwise specified.

### Example 1

Synthesis of N-[3-(4-Phenyl-1H-imidazol-2-yl)-1H-pyrazol-4-yl]-acetamide

### 1A. Synthesis of 4-Nitro-1H-pyrazole-3-carboxylic acid ethyl ester

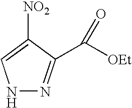

Thionyl chloride (3.8 ml, 52.5 mmol) was added cautiously to a stirred, ice-cold mixture of 4-nitropyrazole-3-carboxylic acid (7.5 g, 47.7 mmol) in EtOH (150 ml), the mixture stirred at ambient temperature for 1 hour then heated at reflux for 3 hours. The reaction mixture was cooled, evaporated in vacuo then azeotroped with toluene to give 4-nitro-1H-pyrazole-3-carboxylic acid ethyl ester (8.8 g).

### 1B. Synthesis of 1-(4-methoxy-benzyl)-4-nitro-1H-prazole-3-carboxylic acid ethyl ester

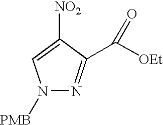

To a solution of 4-nitro-1H-pyrazole-3-carboxylic acid ethyl ester (8.8 g, 47.5 mmol) in MeCN (100 ml) was added K₂CO₃ (7.9 g, 57.0 mmol) followed by 4-methoxybenzyl chloride (7.1 ml, 52.3 mmol) and the mixture stirred at ambient temperature for 20 hours. The mixture was evaporated in vacuo, the residue partitioned between EtOAc and 2M aqueous hydrochloric acid and the organic portion washed with saturated aqueous sodium hydrogen carbonate, dried (MgSO₄) and evaporated in vacuo. The residue was purified by flash column chromatography [SiO₂, EtOAc-hexane (1:4)] to give 1-(4-methoxy-benzyl)-4-nitro-1H-pyrazole-3-carboxylic acid ethyl ester (11 g) as a colourless gum.

### 1C. Synthesis of 4-Amino-1-(4-methoxy-benzal)-1H-pyrazole-3-carboxylic acid ethyl ester

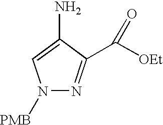

A mixture of 1-(4-methoxy-benzyl)-4-nitro-1H-pyrazole-3-carboxylic acid ethyl ester (1 g) and 10% Pd/C (100 mg) in EtOH (10 ml) was stirred under an atmosphere of hydrogen at ambient temperature and pressure for 3 hours. The catalyst was removed by filtration through Celite and the filtrate evaporated in vacuo to give 4-amino-1-(4-methoxy-benzyl)-1H-pyrazole-3-carboxylic acid ethyl ester (830 mg) as a purple gum.

### 1D. Synthesis of 4-Acetylamino-1-(4-methoxy-benzyl)-1H-pyrazole-3-carboxylic acid ethyl ester

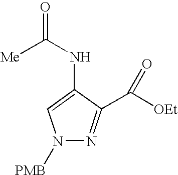

Acetic anhydride (1 ml) was added to a stirred solution of 4-amino-1-(4-methoxy-benzyl)-1H-pyrazole-3-carboxylic acid ethyl ester (1 g) in pyridine (10 ml) and the mixture stirred at ambient temperature for 16 hours. The reaction mixture was evaporated in vacuo, the residue partitioned between EtOAc and 2M hydrochloric acid and the organic portion dried (MgSO₄) and concentrated under reduced pressure to give 4-acetylamino-1-(4-methoxy-benzyl)-1H-pyrazole-3-carboxylic acid ethyl ester (1.2 g) as a pink solid.

### 1E. Synthesis of 4-Acetylamino-1-(4-methoxy-benzyl)-1H-pyrazole-3-carboxylic acid

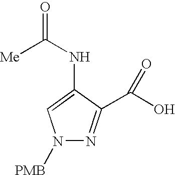

A mixture of 4-acetylamino-1-(4-methoxy-benzyl)-1H-pyrazole-3-carboxylic acid ethyl ester (470 mg, 1.5 mnol) in TBF/water (1:1, 20 ml) was treated with lithium hydroxide monohydrate (70 mg, 1.6 mmol) and stirred at ambient temperature for 16 hours. The volatiles were removed in vacuo and the remaining aqueous solution extracted with Et₂O. The aqueous layer was acidified with 2M hydrochloric acid, then extracted with EtOAc (2×20 ml). The combined EtOAc layers were dried (MgSO₄) and evaporated in vacuo to give 4-acetylamino-1-(4-methoxy-benzyl)-1H-pyrazole-3-carboxylic acid (370 mg) as a white solid.

### 1F. Synthesis of 4-Acetylamino-1-(4-methoxy-benzyl)-1H-pyrazole-3-carboxylic acid 2-oxo-2-phenyl-ethyl ester

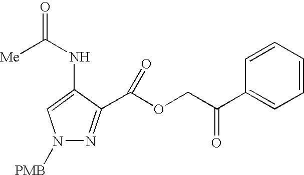

To a stirred solution of 4-acetylamino-1-(4-methoxy-benzyl)-1H-pyrazole-3-carboxylic acid (300 mg, 0.95 mmol) in EtOH/water (1:1, 10 ml) was added caesium carbonate (190 mg, 0.57 mmol) followed by 2-bromoacetophenone (210 mg, 1.04 mmol) and the mixture stirred at 80° C. for 3 hours. The reaction mixture was allowed to cool to ambient temperature and the solid formed collected by filtration, washed with EtOH/water (1:1, 5 ml) then dried under vacuum to give 4-acetylamino-1-(4-methoxy-benzyl)-1H-pyrazole-3-carboxylic acid 2-oxo-2-phenyl-ethyl ester (330 mg) as a white solid.

### 1G. Synthesis of N-[1-(4-Methoxy-benzyl)-3-(4-phenyl-1H-imidazol-2-yl)-1H-pyrazol-4-yl]-acetamide

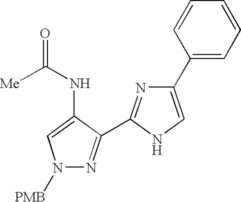

A mixture of 4-acetylamino-1-(4-methoxy-benzyl)-1H-pyrazole-3-carboxylic acid 2-oxo-2-phenyl-ethyl ester (100 mg, 0.24 mmol) and ammonium acetate (380 mg, 4.9 mmol) in p-xylene (5 ml) was heated at 200° C. (100W) for 20 minutes in a CEM Discover™ microwave synthesiser. The reaction mixture was reduced, the residue partitioned between EtOAc and brine and the organic portion dried (MgSO₄) and evaporated in vacuo. The residue was purified by flash column chromatography [SiO₂, EtOAc/hexane (1:2, 1:1)] to give N-[1-(4-methoxy-benzyl)-3-(4-phenyl-1H-imidazol-2-yl)-1H-pyrazol-4-yl]-acetamide (25 mg) as a cream solid. (LC/MS: Rₜ 3.45, [M+H]⁺ 388).

### 1H. Synthesis of N-[3-(4-Phenyl-1H-imidazol-2-yl)-1H-pyrazol-4-yl]-acetamide

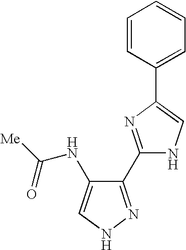

A mixture of N-[1-(4-methoxy-benzyl)-3-(4-phenyl-1H-imidazol-2-yl)-1H-pyrazol-4-yl]-acetamide (20 mg) and anisole (20 μl) in trifluoroacetic acid (1 ml) was heated at 120° C. (50W) for 15 minutes in a CEM Discover™ microwave synthesiser. The reaction mixture was evaporated and then azeotroped with toluene (2×10 ml). The residue was purified by flash column chromatography [SiO₂, EtOAc/hexane (1:1, 1:0)] to give N-[3-(4-phenyl-1H-imidazol-2-yl)-1H-pyrazol-4-yl]-acetamide (10 mg) as an off-white solid. (LC/MS: Rₜ 1.94, [M+H]⁺ 268).

### Example 2

### Synthesis of 2,6-Difluoro-N-[3-(4-phenyl-1H-imidazol-2-yl)-1H-pyrazol-4-yl]-benzamide

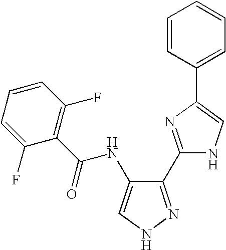

The compound was prepared in a manner analogous to Example 1, but using 2,6-difluorobenzoic acid, EDC and HOBt in place of acetic anhydride and pyridine in step 1D, to give 2,6-difluoro-N-[3-(4-phenyl-1H-imidazol-2-yl)-1H-pyrazol-4-yl]-benzamide (25 mg) as a cream solid. (LC/MS: Rₜ 3.52, [M+H]⁺ 366).

### Example 3

### Synthesis of N-[3-(4-tert-Butyl-1H-imidazol-2-yl)-1H-pyrazol-4-yl]-2,6-difluroro-benzamide

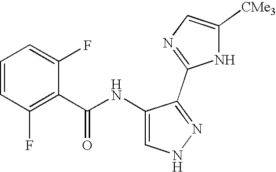

The compound was prepared in a manner analogous to Example 1, but using 2,6-difluorobenzoic acid, EDC and HOBt in place of acetic anhydride and pyridine in step 1D, and 1-bromopinacone in place of 2-bromoacetophenone in step 1F to give the title compound as a glassy solid (10 mg). (LC/MS: Rₜ 2.04, [M+H]⁺ 346.19).

### Example 4

### Synthesis of 2,6-Difluoro-N-{3-[4-(4-fluoro-phenyl)-1H-imidazol-2-yl]-1H-pyrazol-4-yl}-benzamide

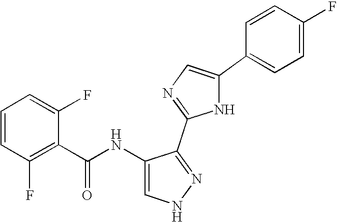

The compound was prepared in a manner analogous to Example 1, but using 2,6-difluorobenzoic acid, EDC and HOBt in place of acetic anhydride and pyridine in step 1D, and 2-bromo-4′-fluoroacetophenone in place of 2-bromoacetophenone in step 1F to give the title compound as a white solid (10 mg). (LC/MS: Rₜ 2.99 [M+H]⁺ 384.13).

### Example 5

### Synthesis of N-{3-[4-(2,4-Difluoro-phenyl)-1H-imidazol-2-yl]-1H-pyrazol-4-yl}-2,6-difluoro-benzamide

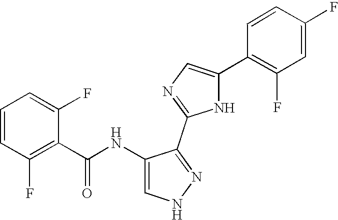

The compound was prepared in a manner analogous to Example 1, but using 2,6-difluorobenzoic acid, EDC and HOBt in place of acetic anhydride and pyridine in step 1D, and 2-chioro-2′,4′-difluoroacetophenone in place of 2-bromoacetophenone in step 1F to give the title compound as a white solid (15 mg). (LC/MS: Rₜ 3.22, [M+H]⁺ 402.13).

### Example 6

### Synthesis of 2,6-Difluoro-N-[3-(4-thioiphen-3-yl-1H-imidazol-2-yl)-1H-pyrazol-4-yl]-benzamide

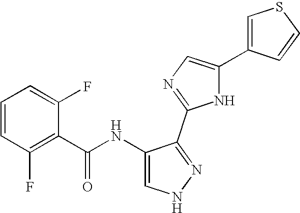

The compound was prepared in a manner analogous to Example 1, but using 2,6-difluorobenzoic acid, EDC and HOBt in place of acetic anhydride and pyridine in step 1D, and 2-bromo-1-(3-thienyl)-1-ethanone in place of 2-bromoacetophenone in step 1F to give the title compound as a beige solid (10 mg). (LC/MS: Rₜ 2.76, [M+H]⁺ 372.10).

### Example 7

### Synthesis of N-{3-[4-(2,3-Dihydro-benzo[1,4]dioxin-6-yl)-1H-imidazol-2-yl]-1H-pyrazol-4-yl}-2,6-difluoro-benzamide

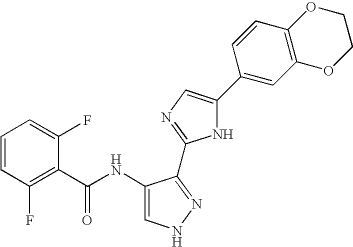

The compound was prepared in a manner analogous to Example 1, but using 2,6-difluorobenzoic acid, EDC and HOBt in place of acetic anhydride and pyridine in step 1D, and 2-bromo-1-(2,3-dihydro-1,4-benzodioxin-6-yl)ethan-1-one in place of 2-bromoacetophenone in step 1F to give the title compound as a beige solid (10 mg). (LC/MS: Rₜ 2.74, [M+H]⁺ 424.19).

### Example 8

### Synthesis of 2,6-Difluoro-N-{3-[4-(4-morpholin-4-yl-phenyl)-1H-imidazol-2-yl]-1H-pyrazol-4-yl}-benzamide

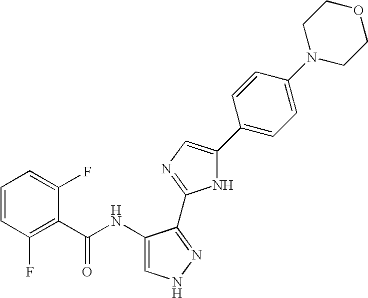

The compound was prepared in a manner analogous to Example 1, but using 2,6-difluorobenzoic acid, EDC and HOBt in place of acetic anhydride and pyridine in step 1D, and 2-bromo-1-(4-morpholinophenyl)-1-ethanone in place of 2-bromoacetophenone in step 1F to give the title compound as a white solid (5 mg). (LC/MS: Rₜ 2.47, [M+H]⁺ 451.20).

### Example 9

### Synthesis of 2,6-Difluoro-N-[3-(4-pyridin-4-yl-1H-imidazol-2-yl)-1H-pyrazol-4-yl]-benzamide

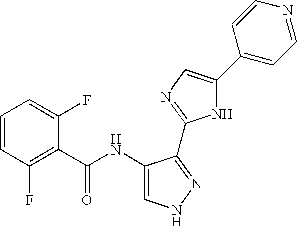

The compound was prepared in a manner analogous to Example 1, but using 2,6-difluorobenzoic acid, EDC and HOBt in place of acetic anhydride and pyridine in step 1D, and 2-bromo-1-(4-pyridinyl)-1-ethanone in place of 2-bromoacetophenone in step 1F to give the title compound as a yellow solid (3 mg). (LC/MS: Rₜ 1.79, [M+H]⁺ 367.11).

### Example 10

### Synthesis of N-(3-{4-[3-(4-Chloro-phenyl)-isoxazol-5-yl]-1H-imidazol-2-yl}-1H-pyrazol-4-yl)-2,6-difluoro-benzamide

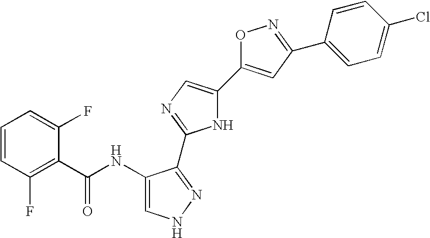

The compound was prepared in a manner analogous to Example 1, but using 2,6-difluorobenzoic acid, EDC and HOBt in place of acetic anhydride and pyridine in step 1D, and 2-bromo-1-[3-(4-chlorophenyl)-5-isoxazolyl)-1-ethanone in place of 2-bromoacetophenone in step 1F to give the title compound as a yellow-brown solid (8 mg). (LC/MS: Rₜ 3.49, [M+H]⁺ 467.10).

### Example 11

### Synthesis of 2,6-Difluoro-N-[3-(5-p-tolyl-1H-imidazol-2-yl)-1H-pyrazol-4-yl]-benzamide

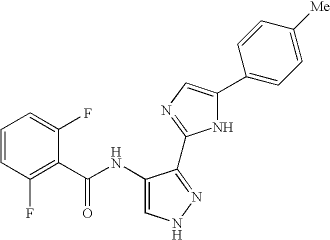

The compound was prepared in a manner analogous to Example 1, but using 2,6-difluorobenzoic acid, EDC and HOBt in place of acetic anhydride and pyridine in step 1D, and 2-bromo-4′-methylacetophenone in place of 2-bromoacetophenone in step 1F to give the title compound as a white solid (8 mg). (LC/MS: Rₜ 2.92, [M+H]⁺ 380).

### Example 12

### Synthesis of 2,6-Difluoro-N-[3-(5-naphthalen-2-yl-1H-imidazol-2-yl)-1H-pyrazol-4-yl]-benzamide

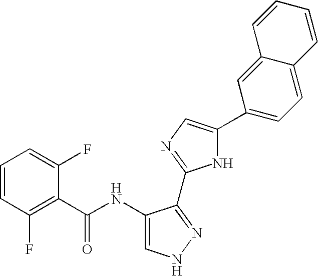

The compound was prepared in a manner analogous to Example 1, but using 2,6-difluorobenzoic acid, EDC and HOBt in place of acetic anhydride and pyridine in step 1D, and α-bromoacetonaphthalene in place of 2-bromoacetophenone in step 1F to give the title compound as a white solid (8 mg). (LC/MS: Rₜ 3.27, [M+H]⁺ 416).

### Example 13

### Synthesis of 2,6-Difluoro-N-{3-[5-(4-pirrolidin-1-yl-phenyl)-1H-imidazol-2-yl]-1H-pyrazol-4-yl}-benzamide

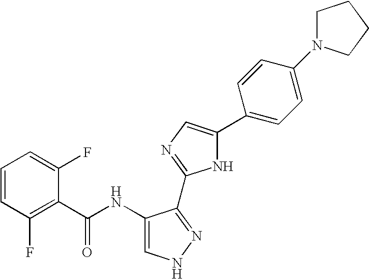

The compound was prepared in a manner analogous to Example 1, but using 2,6-difluorobenzoic acid, EDC and HOBt in place of acetic anhydride and pyridine in step 1D, and α-bromo-4-(1-pyrrolidino)acetophenone in place of 2-bromoacetophenone in step 1F to give the title compound as a white solid (8 mg). (LC/MS: Rₜ 2.77, [M+H]⁺ 435).

### Example 14

### Synthesis of N-[3-(4.5-diphenyl-1H-imidazol-2-yl)-1H-pyrazol-4-yl]-2,6-difluorobenzamide

### 14A. Synthesis of 4-Nitro-1H-pyrazole-3-carboxylic acid methyl ester

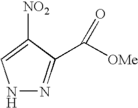

Thionyl chloride (3.8 ml, 52.5 mmol) was added cautiously to a stirred, ice-cold mixture of 4-nitropyrazole-3-carboxylic acid (7.5 g, 47.7 mmol) in MeOH (150 ml), the mixture stirred at ambient temperature for 1 hour then heated at reflux for 3 hours. The reaction mixture was cooled, evaporated in vacuo then azeotroped with toluene to give 4-nitro-1H-pyrazole-3-carboxylic acid ethyl ester (8.8 g).

### 14B. Synthesis of 4-Nitro-1-(tetrahydro-pyran-2-yl)-1H-pyrazole-3-carboxylic acid methyl ester

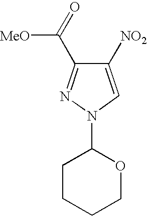

A suspension of 4-nitro-1H-pyrazole-3-carboxylic acid methyl ester (5 g, 29.24 mmol) and p-toluene sulphonic acid (555 mg, 2.92 mmol) in chloroform (100 ml) at 0° C. was treated with 3,4-dihydropyran (4 ml, 43.8 mmol) dropwise. The reaction mixture was allowed to warm to ambient temperature, and then stirred for a further 2 hours. The reaction mixture was diluted with Et₂O, washed sequentially with saturated NaHCO₃ solution and brine. The organic portion was dried (MgSO₄), filtered and evaporated in vacuo. The residue was purified by flash chromatography [silica, EtOAc/Petrol (1:2)] to give 4-nitro-1-(tetrahydro-pyran-2-yl)-1H-pyrazole-3-carboxylic acid methyl ester (7.1 g, 95%) as a colourless oil. (LC/MS: Rₜ 2.86, [M+H]⁺ 256.00).

### 14C. Synthesis of [4-nitro-1-(tetrahydro-pyran-2-yl)-1H-pyrazol-3-yl]-methanol

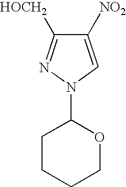

To a stirred solution of 4-nitro-1-(tetrahydro-pyran-2-yl)-1H-pyrazole-3-carboxylic acid methyl ester (10 g, 39.21 mmol) in THF (200 ml) under nitrogen at −78° C. was treated dropwrise with a solution of diisobutylal, im hydride in THP (196 ml, 1M). The reaction mixture was allowed to warm to ambient temperature, and stirred for a further 2 hours. The reaction mixture was diluted with Et₂O, and then quenched with water. The suspension was filtered through celite. The organic portion was dried (MgSO₄), evaporated in vacuo to give [4-nitro-1-(tetrahydro-pyran-2-yl)-1H-pyrazol-3-yl]-methanol as a light brown oil (6 g, 60%).

### 14D. Synthesis of 4-nitro-1-(tetrahydro-pyran-2-yl)-1H-pyrazole-3-carbaldehyde

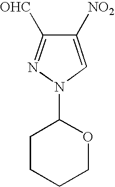

To a stirred solution of [4-nitro-1-(tetrahydro-2-pyran-2-yl)-1H-pyrazol-3-yl]-methanol (6 g, 26.43 mmol) in acetone (60 ml) was added manganese dioxide (22.98 g, 264.3 mmol). The resultant black suspension was heated at 55° C. for 3 hours. The reaction mixture was filtered through celite, and the filtrate evaporated in vacuo to give 4-nitro-1-(tetrahydro-pyran-2-yl)-1H-pyrazole-3-carbaldehyde as a purple/brown viscous oil (4 g, 67%).

### 14E. Synthesis of 3-(4,5-diphenyl-1H-imidazol-2-yl)-4-nitro-1-(tetrahydro-pyran-2-yl)-1H-pyrazole

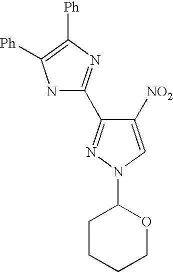

A solution of 4-nitro-1-(tetrahydro-pyran-2-yl)-1H-pyrazole-3-carbaldehyde (0.5 g, 2.22 mmol) and benzil (747 mg, 3.56 mmol) in ethanol (10 ml) and methanolic atnmonia (2N, 10 ml) was stirred at anbient temperature for 2 days. The suspension was filtered, the filtrate was evaporated in vacuo, and purified by flash column chromatography [silica, EtOAc/Petrol (1:2, 1:1)] to give 3-(4,5-diphenyl-1H-imidazol-2-yl)-4-nitro-1-(tetrahydro-pyran-2-yl)-1H-pyrazole as a yellow solid (550 mg, 60%). (LC/MS: Rₜ 3.68, [M+H]⁺ 416.27).

### 14F. Synthesis of 3-(4,5-diphenyl-1H-imidazol-2-yl)-1-(tetrahydro-pyran-2-yl)-1H-pyrazol-4-ylamine

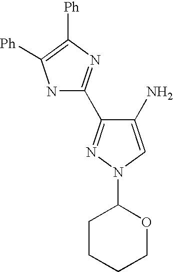

To a stirred solution of 3-(4,5-diphenyl-1H-imidazol-2-yl)-4-nitro-1-(tetrahydro-pyran-2-yl)-1H-pyrazole (450 mg, 1.08 mmol) and ammonium formate (684 mg, 10.84 mmol) in ethanol (10 ml) and water (1 ml) under nitrogen was added palladium on carbon (10%, 25 mg). The reaction mixture was heated at 60° C. for 1 hour. The suspension was filtered through celite, and the filtrate was partitioned between EtOAc and water. The organic portion was dried (NgSO₄), filtered and evaporated in vacuo to give 3-(4,5-diphenyl-1H-imidazol-2-yl)-1-(tetrahydro-pyran-2-yl)-1H-pyrazol-4-ylamine as a pink/purple viscous oil (400 mg, 96%). (LC/MS: Rₜ 2.56, [M+H]⁺ 386.21)

### 14G. Synthesis of N-[3-(4,5-diphenyl-1H-imidazol-2-yl)-1-(tetrahydro-pyran-2-yl)-1H-pyrazol-4-yl]-2,6-difluoro-benzamide

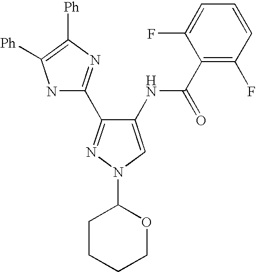

To a solution of 3-(4,5-diphenyl-1H-imidazol-2-yl)-1-(tetrahydro-pyran-2-yl)-1H-pyrazol-4-ylamine (400 mg, 1.04 mmol) in dichloromethane (10 ml) was added EDC (239 mg, 1.25 mmol), HOBt (168 mg, 1.25 mmol) and 2,6-difluorobenzoic acid (164 mg, 1.04 mmol). The reaction mixture was stirred at ambient temperature for 4 hours, and then partitioned between EtOAc and NaOH solution (2N). The organic portion was dried (MgSO₄), filtered and evaporated in vacuo. The residue was purified by flash column chromatography [silica, EtOAc/Petrol (1:4, 1:1)] to give 3-(4,5-diphenyl-1H-imidazol-2-yl)-1-(tetrahydro-pyran-2-yl)-1H-pyrazol-4-ylamine as a white solid (230 mg, 42%). (LC/MS: Rₜ 3.91, [M+H]⁺ 526.18)

### 14H. Synthesis of N-[3-(4,5-diphenyl-1H-imidazol-2-yl)-1H-pyrazol-4-yl]-2,6-difluorobenzamide

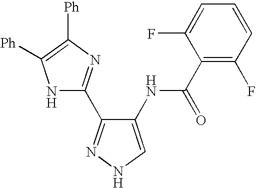

To a suspension of N-[3-(4,5-diphenyl-1H-imidazol-2-yl)-1-(tetrahydro-pyran-2-yl)-1H-pyrazol-4-yl]-2,6-difluoro-benzamide (230 mg, 0.44 mmol) in ethanol (10 ml) was added TsOH (167 mg, 0.88 mmol). The reaction mixture was heated at 60° C. for 1 hour. The reaction mixture was diluted with EtOAc, washed with NaOH solution (2N, ×2) and then brine. The organic portion was dried (MgSO₄), filtered, and evaporated in vacuo. The residue was triturated with Et₂O/Petrol (1:1), filtered and N-[3-(4,5-diphenyl-1H-imidazol-2-yl)-1H-pyrazol-4-yl]-2,6-difluorobenzamide was collected as a white solid (110 mg, 57%). (LC/MS: Rₜ 3.49, [M+H]⁺ 442.09)

### Example 15

### Synthesis of 2,6-difluoro-N-[3-(4-methyl-5-phenyl-1H-imidazol-2-yl)-1H-pyrazol-4-yl]-benzamide

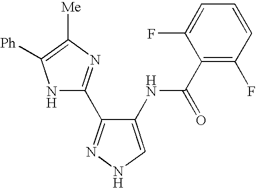

The compound was prepared in an analogous manner to N-[3-(4,5-diphenyl-1H-imidazol-2-yl)-1H-pyrazol-4-yl]-2,6-difluorobenzamide (Example 14) but using phenyl-propane dione in place of benzil in step 14E. (LC/MS: Rₜ 2.56, [M+H]⁺380.06)

### Example 16

### Synthesis of N-[3-(4-Allyl-5-phenpl-1H-imidazol-2-yl)-1H-pyrazol-4-yl]-2,6-difluoro-benzamide

### 16A. Synthesis of 4-(2,6-Difluoro-benzoylamino)-1-(4-methoxy-benzyl)-1H-pyrazole-3-carboxylic acid (2-oxo-2-phenyl-ethyl)-amide

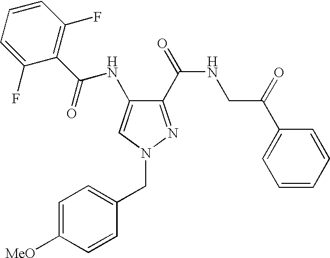

To a stirred solution of 4-(2,6-difluoro-benzoylamino)-1-(4-methoxy-benzyl)-1H-pyrazole-3-carboxylic acid (150 mg; 0.4 mmol), 1-(3-dimethylaminopropyl)-3-ethylcarbodiimide hydrochloride (90 mg; 0.46 mmnol) and 1-hydroxybenzotriazole (65 mg; 0.46 mmol) in 5 ml of N,N-dimethylformamide was added triethylamine (65 μl; 0.46 mmol) followed by 2-aminoacetophenone hydrochloride (80 mg; 0.46 mmol). The reaction mixture was left at room temperature overnight and then evaporated. The residue was partitioned between ethyl acetate (20 ml) and 2M hydrochloric acid (20 ml), the ethyl acetate layer was separated, washed with saturated sodium hydrogen carbonate solution, dried (MgSO₄), filtered and evaporated. The crude product was purified by flash column chromatography, using gradient elution 20% ethyl acetate/hexane to 100% ethyl acetate. Evaporation of product containing fractions gave 105 mg of product as a colourless gum. (LC/MS: Rₜ 3.58, [M+H]⁺ 505).

### 16B. Synthesis of N-[3-(4-Allyl-5-phenyl-1H-imidazol-2-yl)-1-(4-methoxy-benzyl)-1H-pyrazol-4-yl]-2,6-difluoro-benzamide

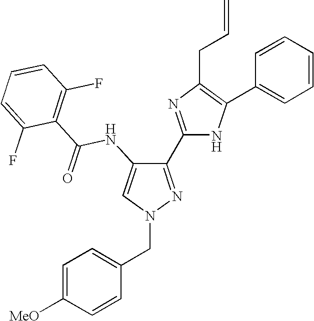

A 60% suspension of sodium hydride in mineral oil (6 mg; 0.12 mmol) was added to a stirred solution of 4-(2,6-difluoro-benzoylamino)-1-(4-methoxy-benzyl)-1H-pyrazole-3-carboxylic acid (2-oxo-2-phenyl-ethyl)-amide (50 mg; 0.1 mmol) in 2 ml of dry DMF then stirred at room temperature for 15 minutes. Allyl bromide (11 μl; 0.11 mmol) was added and the reaction stirred for a further 30 minutes then evaporated. The crude material was dissolved in acetic acid (1 ml), treated with ammonium acetate (100 mg) then heated at 150° C. (100 W) for 20 minutes in a CEM Discover microwave synthesiser. The reaction was evaporated then co-evaporated vith toluene (2×10 ml). The crude product was purified by flash column chromatography, eluting with 1:3 ethyl acetate/hexane. Evaporation of product containing fractions gave 16 mg of product as an off-white solid. (LC/MS: Rₜ 3.76, [M+H]⁺ 526).

### 16C. Synthesis of N-[3-(4-Allyl-5-phenyl-1H-imidazol-2-yl)-1H-pyrazol-4-yl]-2,6-difluoro-benzamide

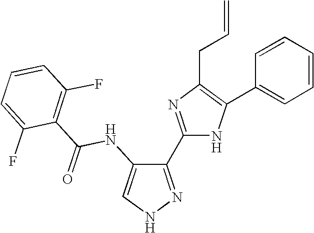

A solution of N-[3-(4-allyl-5-phenyl-1H-imidazol-2-yl)-1-(4-methoxy-benzyl)-1H-pyrazol-4-yl]-2,6-difluoro-benzamide (15 mg) and anisole (20 μl) in trifluoroacetic acid (500 μl) was heated at 150° C. (100 W) for 20 minutes in a CEM Discover microwave synthesiser. The reaction was evaporated then co-evaporated with toluene (2×10 ml). The crude product was purified by flash column chromatography, eluting with 1:1 ethyl acetate/hexane. Evaporation of product containing fractions gave 5 mg of product as a cream solid. (LC/MS: Rₜ 3.02, [M+H]⁺ 406).

### Example 17

### Synthesis of 2,6-Difluoro-N-{3-[4-(3-hydroxy-propyl)-5-phenyl-1H-imidazol-2-yl]-1H-pyrazol-4-yl}-benzamide

### 17A. Synthesis of 4-Nitro-1H-pyrazole-3-carboxylic acid (2-oxo-2-phenyl-ethyl)-amide

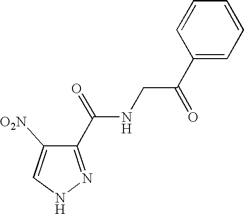

To a stirred solution of 4-nitropyrazole-3-carboxylic acid (1.06 g; 6.75 mmol), 1-(3-dimethylaminopropyl)-3-ethylcarbodiimide hydrochloride (1.55 g; 8.1 mmol) and 1-hydroxybenzotriazole (1.1 g; 8.1 mmol) in 20 ml of N,N-dimethylformamide was added triethylamine (1.9 ml; 0.46 mmol) followed by 2-aminoacetophenone hydrochloride (1.16 g; 6.75 mmol). The reaction mixture was left at room temperature overnight and then evaporated. The residue was partitioned between ethyl acetate (50 ml) and water (50 ml), the ethyl acetate layer was separated, dried (MgSO₄), filtered and evaporated to give the product as a pale yellow solid. (LC/MS: Rₜ 2.98, [M+H]⁺ 275).

### 17B. Synthesis of 4-Nitro-1-(tetrahydro-pyran-2-yl)-1H-pyrazole-3-carboxylic acid (2-oxo-2-phenyl-ethyl)-amide

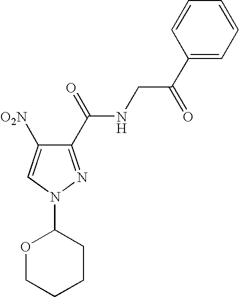

4-Nitro-1H-pyrazole-3-carboxylic acid (2-oxo-2-phenyl-ethyl)-amide (780 mg; 2.8 mmol) was suspended in dichloromethane (20 ml), treated with 2,3-dihydropyran (400 μl; 4.2 mmol) and a catalytic amount of p-toluenesulphonic acid then stirred at room temperature overnight. The reaction was diluted with dichloromethane (20 ml), washed with saturated sodium hydrogen carbonate solution, dried (MgSO₄), filtered and evaporated to give 1.1 g of the product as a pale brown gum. (LC/MS: Rₜ 2.9, [M+H]⁺ 359).

### 17C. Synthesis of 4-Nitro-1-(tetrahydro-pyran-2-yl)-1H-pyrazole-3-carboxylic acid (1-benzoyl-but-3-enyl)-amide

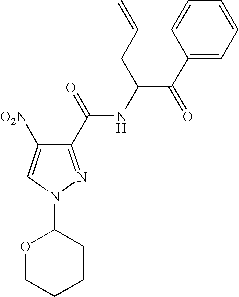

A 60% suspension of sodium hydride in mineral oil (150 mg; 3.7 mmol) was added to a stirred solution of 4-nitro-1-(tetrahydro-pyran-2-yl)-1H-pyrazole-3-carboxylic acid (2-oxo-2-phenyl-ethyl)-amide (1.1 g; 3.07 mmol) in 40 ml of dry DMF at −40° C. then stirred for 30 minutes. Allyl bromide (290 μl; 3.4 mmol) was added, the reaction was stirred for a further 60 minutes then evaporated. The residue was partitioned between ethyl acetate (100 ml) and brine (100 ml), the ethyl acetate layer was separated, dried (MgSO₄), filtered and evaporated. The crude product was purified by flash column chromatography, eluting with 1:2 then 2:3 ethyl acetate/hexane. Evaporation of product containing fractions gave 520 mg of the required compound. (LC/MS: Rₜ 3.24, [M+H]⁺ 399).

### 17D. Synthesis of 3-(4-Allyl-5-phenyl-1H-imidazol-2-yl)-4-nitro-1H-pyrazole

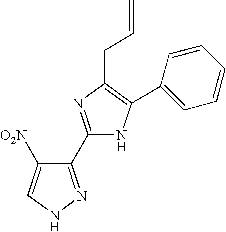

A mixture of 4-nitro-1-(tetrahydro-pyran-2-yl)-1H-pyrazole-3-carboxylic acid (1-benzoyl-but-3-enyl)-amide (1 g) and ammonium acetate (1 g) in acetic acid (6 ml) was heated at 120° C. (100 W) for 20 minutes in a CEM Discover microwave synthesiser. The reaction was evaporated then co-evaporated with toluene (2×20 ml). The residue was treated with brine (50 ml) the pH was adjusted to 6, and the mixture was then extracted with dichloromethane (50 ml). The dichloromethane layer was separated, dried (MgSO₄), filtered and evaporated to give 560 mg of the required product. (LC/MS: Rₜ 2.19, [M+H]⁺ 296).

### 17E. Synthesis of 3-[2-(4-Nitro-1H-pyrazol-3-yl)-5-phengl-1H-imidazol-4-yl]-propan-1-ol

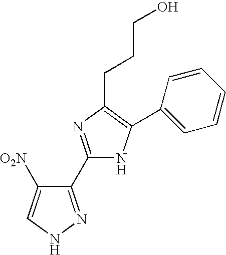

Borane-dimethylsulphide complex (2M solution in anhydrous THF) was added to a stirred solution of 3-(4-allyl-5-phenyl-1H-imidazol-2-yl)-4-nitro-1H-pyrazole (50 mg; 0.17 mmol) in anhydrous THF (5 ml) at 0° C. then allowed to warm to room temperature and stirred for a further 3 hours. The reaction was treated with a mixture of 2M sodium hydroxide (500 μl ), 30% aqueous hydrogen peroxide sol-ution (500 μl) and water (2 ml) and stirring continued for a further 30 minutes. The reaction mixture was diluted with ethyl acetate (20 ml) and 2M ammonium chloride solution (20 ml). The ethyl acetate layer was separated, dried (MgSO₄), filtered and evaporated to give 35 mg of the required product. (LC/MS: Rₜ 1.83, [M+H]⁺ 314).

### 17F. Synthesis of 2,6-Difluoro-N-{3-[4-(3-hydroxy-propyl)-5-phenyl-1H-imidazol-2-yl]-1H-pyrazol-4-yl}-benzamide

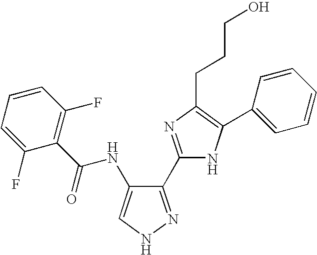

10% Palladium on carbon (20 mg) was added to a solution of 3-[2-(4-nitro-1H-pyrazol-3-yl)-5-phenyl-1H-imidazol-4-yl]-propan-1-ol (140 mg) in ethanol then hydrogenated at room temperature and pressure for 3 hours. The catalyst was removed by filtration and the filtrate evaporated. The crude material was dissolved in DMF (5 ml), treated with 2,6-difluorobenzoic acid (80 mg; 0.5 mmol), 1-(3-dimethylaminopropyl)-3-ethylcarbodiimide hydrochloride (105 mg; 0.54 mmol) and 1-hydroxybenzotriazole (75 mg; 0.54 mmol), left at room temperature overnight, and then evaporated. The residue was partitioned between ethyl acetate (20 ml) and brine (20 ml), the ethyl acetate layer was separated, dried (MgSO₄), filtered and evaporated. The crude product was purified by flash column chromatography, eluting with ethyl acetate. Evaporation of product containing fractions gave 22 mg of the required compound as a brown solid. (LC/MS: Rₜ 2.28, [M+H]⁺ 424).

### Example 18

### Synthesis of 2-[4-(2,6-Difluoro-benzoylamino)-1H-pyrazol-3-yl]-5-isobutyl-1H-imidazole-4-carboxylic acid ethyl ester

### 18A. Synthesis of 4-methyl-3-oxo-2-(trphenyl-lambda*5*-phosphanylidene)-pentanoic acid ethyl ester

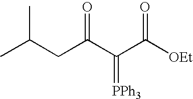

Isovaleryl chloride (385 μl; 3.16 mmol) was added dropwise to a stirred solution of (carbethoxymethylene)triphenylphosphorane (1 g; 2.87 mmol) and diisopropylethylamine (550 μl; 3.16 mmol) in dichloromethane (20 ml) then stirred at room temperature for 2 hours. The reaction was diluted with dichloromethane (20 ml), washed with 1M hydrochloric acid (20 ml) then saturated sodium bicarbonate solution (20 ml). The organic layer was separated, dried (MgSO₄), filtered and evaporated to give 1.26 g of 4-Methyl-3-oxo-2-(triphenyl-lambda*5*-phosphanylidene)-pentanoic acid ethyl ester as a yellow solid. (LC/MS: Rₜ 3.67, [M+H]⁺ 433).

### 18B. Synthesis of 4-methyl-2,3-dioxo-pentanoic acid ethyl ester

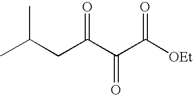

A mixture of 4-methyl-3-oxo-2-(triphenyl-lambda*5*-phosphanylidene)-pentanoic acid ethyl ester (1.2 g; 2.8 mmol) and oxone (2.1 g; 3.36 mmol) in TMF/water (10 ml:10 ml) was stirred at room temperature overnight. The reaction was diluted with ethyl acetate (20 ml), washed with brine (20 ml), dried (MgSO₄), filtered and evaporated. The crude material was purified by flash column chromatography on silica gel eluting with 1:3 ethyl acetate/petroleum ether. Product containing fractions were combined and evaporated to give 425 mg of 4-methyl-2,3-dioxo-pentanoic acid ethyl ester as a mobile yellow oil.

### 18C. Spnthesis of 4-(2,6-difluoro-benzylamino)1-(4-methoxy-benzyl)-1H-pyrazole-3-carboxylic acid methyl ester

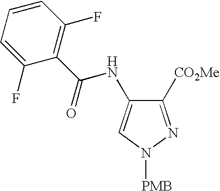

To a stirred solution of 4-amino-1-(4-methoxy-benzyl)-1H-pyrazole-3-carboxylic acid ethyl ester (8.9 g, 34.10 mmoles) in dichloromethane (150 ml) was added EDC (7.8 g, 40.92 mmoles), HOBt (5.5 g, 40.92 mmoles) and 2,6-difluorobenzoic acid (5.4 g, 34.10 mmoles). The reaction mixture was stirred at ambient temperature for 48 hrs, and then partitioned between EtOAc and NaOH solution (2N). The organic portion was washed with brine, dried (MgSO₄), filtered and evaporated in vacuo. The residue was purified by flash column chromatography (Biotage SP4, 2×40M, flow rate 40 ml/min, gradient 1:4 EtOAc/Petrol to EtOAc) to give an off-white solid (6.0 g, 44%). (LC/MS:Rₜ 3.16, [M+H]⁺ 402.00).

### 18D. Synthesis of 2,6-difluoro-N-[3-hydroxymethyl-1-(4-methoxy-benzyl)-1H-pyrazol-4-yl]-benzamide

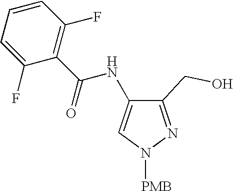

A stirred solution of 4-(2,6-difluoro-benzylamino)1-(4-methoxy-benzyl)-1H-pyrazole-3-carboxylic acid methyl ester (6.0 g, 14.96 mmoles) in TBF (150 ml) under nitrogen at −78° C. was treated dropwise with a solution of diisobutylaluminium hydride in THF (150 ml, 1M). The reaction mixture was stirred at −78° C. for 1 hour and then warmed to 0° C. in an ice-water bath. A saturated aqueous solution of sodium sulphate (200 ml) was added to the reaction mixture. The suspension was filtered through celite. The filtrate was partitioned between EtOAc and brine. hie organic portion was dried (MgSO₄), filtered and evaporated in vacuo to give 2,6-difluoro-N-[3-hydroxymethyl-1-(4-methoxy-benzyl)-1H-pyrazol-4-yl]-benzamide as a yellow solid (4.6 g, 82%). (LC/MS: Rₜ 2.48, [M+H]⁺ 374.04).

### 18E. Synthesis of 2,6-difluoro-N-[3-formyl-1-(4-methoxy-benzyl)-1H-pyrazol-4-yl]-benzamide

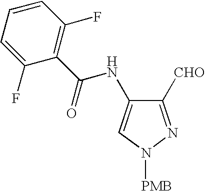

To a stirred solution of 2,6-difluoro-N-[3-hydroxymethyl-1-(4-methoxy-benzyl)-1H-pyrazol-4-yl]-benzamide (4.6 g, 12.33 mmoles) in acetone (100 ml) was added MnO₂ (6.9 g, 79.37 mmoles). The resultant black suspension was stirred at ambient temperature for 24 hours. The reaction mixture was filtered through celite, and the fitrate evaporated in vacuo. The residue was purified by flash column chromatography (Biotage SP4, 40S, flow rate 30 ml/min, gradient 1:4 EtOAc/Petrol to 4:1 EtOAc/Petrol) to give 2,6-difluoro-N-[3-formyl-1-(4-methoxy-benzyl)-1H-pyrazol-4-yl]-benzamide as a yellow-brown liquid (2.2 g, 48%). (LC/MS: Rₜ 3.18, [M+H]⁺ 372.04).

### 18F. Synthesis of 2-[4-(2,6-difluorobenzoylamino)-1-(4-methoxy-benzyl)-1H-pyrazol-3-yl]-5-isobutyl-1H-imidazole-4-carboxylic acid ethyl ester

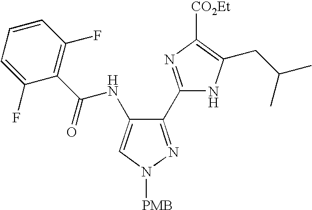

A mixture of 4-methyl-2,3-dioxo-pentanoic acid ethyl ester (100 mg), 2,6-difluoro-N-[3-formyl-1-(4-methoxy-benzyl)-1H-pyrazol-4-yl]-benzamide (25 mg) and ammonium acetate (100 mg) was heated at 100° C. (100W) for 10 minutes in a CEM discover microwave synthesiser. The reaction mixture was evaporated and re-evaporated with toluene. The residue was partitioned between ethyl acetate (15 ml) and brine (15 ml), the ethyl acetate layer was separated, dried (MgSO₄), filtered and evaporated. The crude material was purified by flash column chromatography on silica gel eluting with 1:3 then 1:2 ethyl acetate/petroleum ether. Product containing fractions were combined and evaporated to give 33 mg of 2-[4-(2,6-difluorobenzoylamino)-1-(4-methoxy-benzyl)-1H-pyrazol-3-yl]-5-isobutyl-1H-imidazole-4-carboxylic acid ethyl ester. (LC/MS: Rₜ 3.80, [M+H]⁺ 538).

### 18G. Synthesis of 2-[4-(2,6-difluoro-benzoylamino)-1H-pnrazol-3-yl]-5-isobutyl-1H-imidazole-4-carboxylic acid ethyl ester

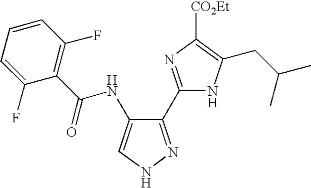

A solution of 2-[4-(2,6-difluorobenzoylamino)-1-(4-methoxy-benzyl)-1H-pyrazol-3-yl]-5-isobutyl-1H-imidazole-4-carboxylic acid ethyl ester (30 mg) and anisole (30 μl) in trifluoroacetic anhydride (500 μl) was heated at 150° C. (100 W) for 10 minutes in a CEM discover microwave synthesiser. The reaction mixture was evaporated and re-evaporated with toluene. The crude material was purified by flash column chromatography on silica gel eluting with 1:2 then 1:1 ethyl acetate/petroleum ether. Product containing fractions were combined and evaporated to give 20 mg of 2-[4-(2,6-difluoro-benzoylamino)-1H-pyrazol-3-yl]-5-isobutyl-1H-imidazole-4-carboxylic acid ethyl ester. (LC/MS: Rₜ 3.24, [M+H]⁺ 418).

| | | Prepared using | Differences to | |
|---|---|---|---|---|
| Example | | method | General | |
| Number | Structure | analogous to: | Procedure | LCMS |
| 19 | 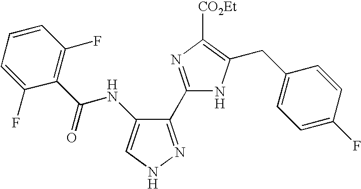 | Example 18 | Starting from 4- fluorophenylacetic acid | [M + H]⁺ = 470 Rₜ = 3.25 |

### Example 20

### Synthesis of 2,6-difluoro-N-{3-[5-isobutyl-4-(morpholine-4-carbonyl)-1H-imidazol-2-yl]-1H-pyrazol-4-yl}-benzamide

### 20A. Synthesis of 2-[4-(2,6-difluorobenzoylamino)-1-(4-methoxy-benzyl)-1H-pyrazol-3-yl]-5-isobutyl-1H-imidazole-4-carboxylic acid

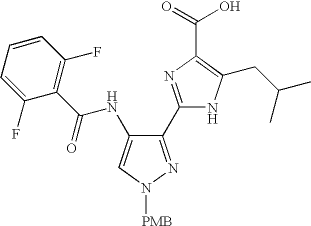

A solution of 2-[4-(2,6-difluorobenzoylamino)-1-(4-methoxy-benzyl)-1H-pyrazol-3-yl]-5-isobutyl-1H-imidazole-4-carboxylic acid ethyl ester (250 mg) in methanol (5 ml) was treated with 2M sodium hydroxide solution (5 ml) then heated at 65° C. over the weekend. The methanol was evaporated and the pH adjusted to 5 with 2M hydrochloric acid. The aqueous was extracted with ethyl acetate and the ethyl acetate layer was dried (MgSO₄), filtered and evaporated to give 160 mg of 2-[4-(2,6-difluorobenzoylamino)-1-(4-methoxy-benzyl)-1H-pyrazol-3-yl]-5-isobutyl-1H-imidazole-4-carboxylic acid as a cream foam. (LC/MS: Rₜ 3.27, [M+H]⁺ 510).

### 20B. Synthesis of 2,6-difluoro-N-[3-[5-isobutyl-4-(morpholine-4-carbonyl)-1H-imidazol-2-yl]-1-(4-methoxy-benzyl)-1H-pyrazol-4-yl]-benzamide

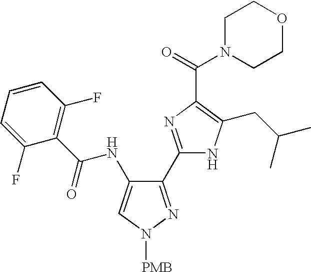

A solution of 2-[4-(2,6-difluorobenzoylamino)-1-(4-methoxy-benzyl)-1H-pyrazol-3-yl]-5-isobutyl-1H-imidazole-4-carboxylic acid (50 mg; 0.12 mmol), 1-(3-dimethylaminopropyl)-3-ethylcarbodiimide hydrochloride (30 mg; 0.14 mmol) and 1-hydroxybenzotriazole (65 mg; 0.14 mmol) and morpholine (15 μl) in 5 ml of N,N-dimethylformamide was left at room temperature overnight and then evaporated. The residue was partitioned between ethyl acetate (20 ml) and 2M hydrochloric acid (20 ml), the ethyl acetate layer was separated, washed with saturated sodium hydrogen carbonate solution (20 ml), dried (MgSO₄), filtered and evaporated. The crude product was purified by flash column chromatography, using 1:1 then 2:1 ethyl acetate/hexane. Evaporation of product containing fractions gave 25 mg of product as a colourless gum.

### 20C. Synthesis of 2,6-difluoro-N-{3-[5-isobutyl-4-(morpholine-4-carbonyl)-1H-imidazol-2-yl]-1H-pyrazol-4-yl}-benzamide

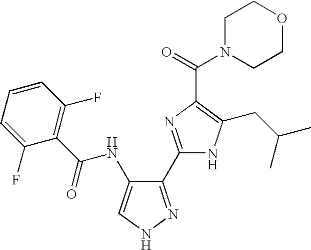

A solution of 2,6-Difluoro-N-[3-[5-isobutyl-4-(morpholine-4-carbonyl)-1H-imidazol-2-yl]-1-(4-methoxy-benzyl)-1H-pyrazol-4-yl]-benzamide (25 mg) and anisole (30 μl) in trifluoroacetic acid (500 μl) was heated at 120° C. (100 W) for 10 minutes in a CEM discover microwave synthesiser. The reaction mixture was evaporated and re-evaporated with toluene. The crude material was triturated with 1:1 diethyl ether/hexane and the product collected by filtration as a cream solid (11.5 mg). (LC/MS: Rₜ 2,61, [M+H]⁺ 459).

| | | Prepared using | | |
|---|---|---|---|---|
| | | method | | |
| | | analogous to | Differences to | |
| Example | | General | General | |
| Number. | Structure | Procedure XX | Procedure | LCMS |
| 21 | 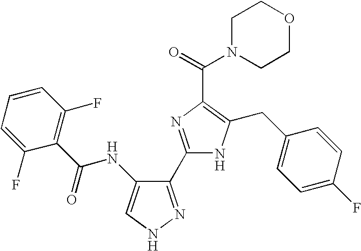 | 18 and 20 | Starting from 4- fluorophenylacetic acid | [M + H]⁺ = 511 Rₜ = 2.79 |
| 22 | 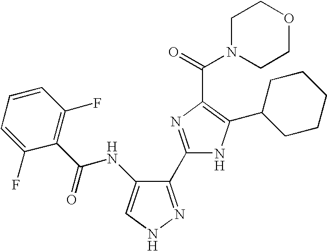 | 18 and 20 | Starting from 4- cyclohexanecarboxylic acid | [M + H]⁺ = 485 Rₜ = 2.82 |
| 23 | 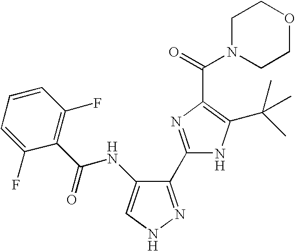 | 18 and 20 | Starting from pivaloyl chloride | [M + H]⁺ = 459 Rₜ = 2.54 |
| 24 | 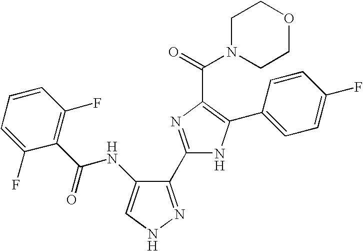 | 18 and 20 | Starting from 4- fluorobenzoic acid | [M + H]⁺ = 497 Rₜ = 2.76 |

### Example 25

### Synthesis of N-[3-(5-benzyl-4-morpholin-4-ylmethyl-1H-imidazol-2-yl)-1H-pyrazol-4-yl]-2,6-difluoro-benzamide

### 25A. Synthesis of (4,4-diethoxy-but-2-ynyl)-benzene

To a solution of 7.9 ml ethyl magnesium bromide (3M solution in THF) in dibutyl ether (20 ml) was added 3-phenyl-1-propyne (2.7 ml; 21.5 mmol), heated at 50° C. for 1 hour, treated with triethylorthoformate (7.2 ml; 47.4 mmol) then allowed to cool to room temperature over 1 hour. The reaction mixture was quenched with saturated ammonium chloride solution (20 ml) then extracted with ethyl acetate (2×20 ml). The combined extracts were dried (MgSO₄), filtered and evaporated to give 2.2 g of (4,4-diethoxy-but-2-ynyl)-benzene as a mobile yellow liquid.

### 25B. Spnthesis of 1,1-diethoxy-4-phenyl-butane-2,3-dione

A solution of (4,4-diethoxy-but-2-ynyl)-benzene (2.15 g; 9. mmol) in acetonitrile/carbon tetrachloride/water (10 ml:10 ml:14 ml) was treated with sodium periodate (8.45 g; 40 mmol) followed by ruthenium chloride (60 mg; 0.3 mmol) then stirred at room temperature overnight. The reaction was diluted with water (50 ml) and ethyl acetate (50 ml) then filtered. The ethyl acetate layer was separated, washed with brine (20 ml), then dried (MgSO₄), filtered and evaporated. The crude product was purified by flash column chromatography, using 1:6 ethyl acetate/hexane. Evaporation of product containing fractions gave 1.28 g of 1,1-diethoxy-4-phenyl-butane-2,3-dione.

### 25C. Synthesis of 4-amino-1-(tetrahydro-pyran-2-yl)-1H-pyrazole-3-carboxylic acid methyl ester

To a stirred solution of 4-nitro-1-(tetrahydro-pyran-2-yl)-1H-pyrazole-3-carboxylic acid methyl ester (16.0 g, 62.75 mmoles, example 14B) and ammonium formate (39.6 g, 627.45 mmoles) and in ethanol (200 ml) and water (20 ml) under nitrogen was added palladium on carbon (10%, 0.8 g). The reaction mixture was heated at 50° C. for 2 hours. The suspension was filtered through celite, and the filtrate was partitioned between EtOAc and water. The organic portion was dried (MgSO₄) to give 4-amino-1-(tetrahydro-pyran-2-yl)-1H-pyrazole-3-carboxylic acid methyl ester as a yellow oil (12.5 g, 89%). (LC/MS: Rₜ 1.84, [M+H]⁺ 226.06).

### 25D. Synthesis of 4-(2,6-difluoro-benzoylamino)1-(tetrahydro-pyran-2-yl-1H-pyrazole-3-carboxylic acid methyl ester

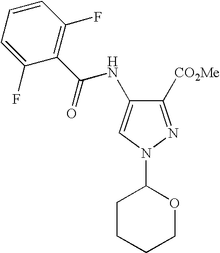

A solution of 4-amino-1-(tetrahydro-pyran-2-yl)-1H-pyrazole-3-carboxylic acid methyl ester (12.5 g, 55.56 mmoles), EDC (18.78 g, 97.96 mmoles), HOBt (13.00 g, 96.30 mmoles) and 2,6-difluorobenzoic acid (12.8 g, 81.01 mmoles) in dichloromethane was stirred at ambient temperature for 24 hours, and then partitioned between EtOAc and NaOH solution (2N). The organic portion was dried (MgSO₄), filtered and evaporated in vacuo. The residue was purified [Biotage SP4, 3×40M, flow rate 40 ml/min, gradient 3:7 EtOAc/Petrol to 2:1 EtOAc/Petrol] to give 4-(2,6-difluoro-benzoylamino)1-(tetrahydro-pyran-2-yl-1H-pyrazole-3-carboxylic acid methyl ester as a white solid (11.3 g, 56%). (LC/MS: Rₜ 3.10, [M+H]⁺ 366.19).

### 25E. Synthesis of 2,6-difluoro-N-[3-hydroxymethyl-1-(tetrahydro-pyran-2-yl)-1H-pyrazol-4-yl]-benzamide

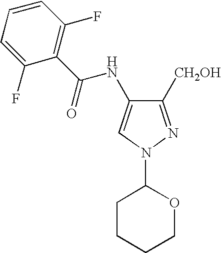

A stirred solution of 4-(2,6-difluoro-benzoylamino)1-(tetrahydro-pyran-2-yl-1H-pyrazole-3-carboxylic acid methyl ester (11.3 g, 30.96 mmoles) in THF (500 ml) under nitrogen at −78° C. was treated dropwise with a solution of diisobutylaluminium hydride in THF (310 ml, 1M). The reaction mixture was stirred at −78° C. for 1 hour and then warmed to 0° C. in an ice-water bath. A saturated aqueous solution of sodium sulphate (300 ml) was added o the reaction mixture. The suspension was filtered through celite. The filtrate was partitioned between EtOAc and brine. The organic portion was dried (MgSO₄), filtered and evaporated in vacuo to give 2,6-difluoro-N-[3-hydroxymethyl-1-(tetrahydro-pyran-2-yl)-1H-pyrazol-4-yl]-benzamide as a white solid (10.14 g, 97%). (LC/MS: Rₜ2.34, [M+H]⁺338.03).

### 25F. Synthesis of 2,6-difluoro-N-[3-formyl-1-(tetrahydro-pnran-2-yl)-1H-pyrazol-4-yl]-benzamide

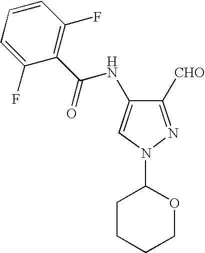

To a stirred solution of 2,6-difluoro-N-[3-hydroxymethyl-1-(tetrahydro-pyran-2-yl)-1H-pyrazol-4-yl]-benzamide (10.14 g, 30.10 mmoles) in acetone (200 ml) was added MnO₂ (52.33 g, 602 mmoles). The resultant black suspension was stirred at ambient temperature for 48 hours. The reaction mixture was filtered through celite, and the fitrate evaporated in vacuo. The residue was purified by flash column chromatography (Biotage SP4, 40M, flow rate 30 ml/min, gradient 1:3 EtOAc/Petrol to 3:2 EtOAc/Petrol) to give 2,6-difluoro-N-[3-formyl-1-(tetrahydro-pyran-2-yl)-1H-pyrazol-4-yl]-benzamide as a creamy solid (7.8 g, 77%). (LC/MS: Rₜ 3.03, [M+H]⁺ 336.03).

### 25G. Synthesis of N-[3-(5-benzyl-4-formyl-1H-imidazol-2-yl)-1-(tetrahydro-pyran-2-yl)-1H-pyrazol-4-yl]-2,6-difluoro-benzamide

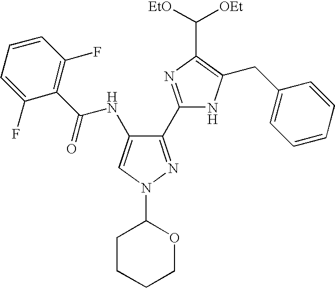

A mixture of 2,6-difluoro-N-[3-formyl-1-(tetrahydro-pyran-2-yl)-1H-pyrazol-4-yl]-benzamide (500 mg) and 1,1-diethoxy-4-phenyl-butane-2,3-dione (500 mg) in 2M ammonia in methanol (20 ml) was stirred at room temperature for 4 hours then evaporated. The crude product was purified by flash column chromatography, using 1:3 then 1:2 ethyl acetate/hexane. Evaporation of product containing fractions gave 205 mg of N-[3-(5-benzyl-4-formyl-1H-imidazol-2-yl)-1-(tetrahydro-pyran-2-yl)-1H-pyrazol-4-yl]-2,6-difluoro-benzamide. (LC/MS: Rₜ 3.38, [M+H]⁺ 492).

### 25H. Synthesis of N-[3-(5-benzyl-4-morpholin-4-ylmethyl-1H-imidazol-2-yl)-1H-pyrazol-4-yl]-2,6-difluoro-benzamide

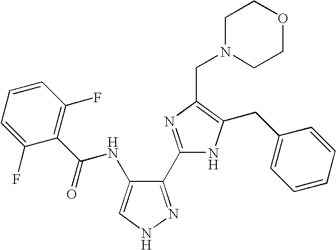

A solution of N-[3-(5-benzyl-4-formyl-1H-imidazol-2-yl)-1-(tetrahydro-pyran-2-yl)-1H-pyrazol-4-yl]-2,6-difluoro-benzamide (50 mg; 0.1 mmol) in TMF (10 ml) was treated with 3 Å molecular sieves (1 g), morpholine (20 μl; 0.2 mmol) and sodium triacetoxyborohydride (65 mg; 0.3 mmol), stirred at room temperature then filtered and evaporated. The crude material was dissolved in ethanol (5 ml), treated with 50 mg of p-toluenesulphonic acid, and was heated at 130° C. (100 W) for 20 minutes in a CEM discover microwave synthesiser. The reaction mixture was evaporated and the residue was partitioned between ethyl acetate (20 ml) and saturated sodium hydrogen carbonate (20 ml). The ethyl acetate layer was separated then dried (MgSO₄), filtered and evaporated. The crude product was purified by flash column chromatography, using 2% then 5% methanol in DCM. Evaporation of fractions 22 to 28 gave 9 mg of N-[3-(5-benzyl-4-morpholin-4-ylmethyl-1H-imidazol-2-yl)-1H-pyrazol-4-yl]-2,6-difluoro-benzamide as a white solid. (LC/MS: Rₜ 2.12, [M+H]⁺479).

### Example 26

### Synthesis of N-[3-(5-benzyl-4-ethoxymethyl-1H-imidazol-2-yl)-1H-pyrazol-4-yl]-2,6-difluoro-benzamide

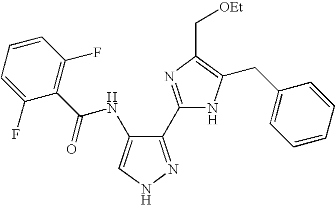

From 25H, evaporation of fractions 7-14 gave 10 mg of N-[3-(5-benzyl-4-ethoxymethyl-1H-imidazol-2-yl)-1H-pyrazol-4-yl]-2,6-difluoro-benzamide as a cream solid. (LC/MS: Rₜ 2,63, [M+H]⁺ 438).

### Example 27

### Synthesis of N-[3-(5-benzyl-4-hydroxymethyl-1H-irnidazol-2-yl)-1H-pyrazol-4-yl]-2,6-difluoro-benzamide

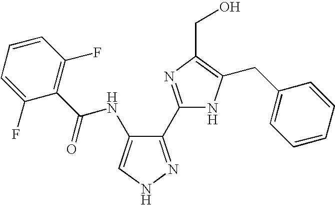

A solution of N-[3-(5-benzyl-4-formyl-1H-imidazol-2-yl)-1-(tetrahydro-pyran-2-yl)-1H-pyrazol-4-yl]-2,6-difluoro-benzamide (50 mg; 0.1 mmol) in THF (10 ml) was treated with sodium borohydride (10 mg), then stirred at room temperature for 2 hours and then evaporated. The residue was partitioned between ethyl acetate (20 ml) and saturated brine (20 ml). The ethyl acetate layer was separated, dried (MgSO₄), filtered and evaporated. The crude material was dissolved in a mixture of concentrated hydrochloric acid/water/dioxan (1 ml:2 ml:2 ml) and heated at 70° C. overnight. The dioxan was removed by evaporation and the residue basified with saturated sodium hydrogen carbonate. The resulting solid was collected by filtration, washed with water and sucked dry to give 25 mg of N-[3-(5-benzyl-4-hydroxymethyl-1H-imidazol-2-yl)-1H-pyrazol-4-yl]-2,6-difluoro-benzamide. (LC/MS: Rₜ 2.15, [M+H]⁺ 410).

### Example 28

### Synthesis of 2,6-difluoro-N-{3-[4-(2-hydroxy-ethyl)-5-isobutyl-1H-imidazol-2-yl]-1H-pyrazol-4-yl}-benzamide

### 28A. Synthesis of 6-methyl-hept-3-yn-1-ol

To a solution of 4.8 ml ethyl magnesium bromide (3M solution in TBE) in THF (20 ml) was added 4-methyl-1-pentyne (2.7 ml; 21.5 mmol). The reaction mixture was stirred at room temperature for 1 hour, then ethylene oxide was bubbled through for 20 minutes. The reaction mixture was stirred at room temperature for a flther hour, then quenched with saturated ammonium chloride solution (20 ml) and extracted with diethyl ether (2×20 ml). The combined extracts were dried (MgSO₄), filtered and evaporated to give 5.3 g of 6-methyl-hept-3-yn-1-ol as a mobile colourless liquid.

### 28B. Synthesis of tert-butyl-dimethyl-(6-methyl-hept-3-ynyloxy)-silane

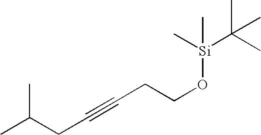

A solution of 6-methyl-hept-3-yn-1-ol (5.1 g; 41 mmol), imidazole (5.6 g; 82 mmol) and tert-butyldimethylsilyl chloride (7.4 g; 49 mmol) in DMF was stirred at room temperature for 2 hours then evaporated. The residue was partitioned between diethyl ether (100 ml) and 1M hydrochloric acid (100 ml). The ether layer was separated, washed with saturated sodium hydrogen carbonate (100 ml), dried (MgSO₄), filtered and evaporated to give 3.9 g of tert-butyl-dimethyl-(6-methyl-hept-3-ynyloxy)-silane as a colourless liquid.

### 28C. Synthesis of 1-(tert-butyl-dimethyl-silanyloxy)-6-methyl-heptane-3,4-dione

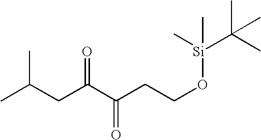

A solution of tert-butyl-dimethyl-(6-methyl-hept-3-ynyloxy)-silane (2 g; 8.3 mmol) in acetonitrile/carbon tetrachloride/water (20 ml:20 ml:28 ml) was treated with sodium periodate (7 g; 33.2 mmol) followed by ruthenium chloride (52 mg) then stirred at room temperature overnight. The reaction mixture was diluted with water (50 ml) and ethyl acetate (50 ml). The ethyl acetate layer was separated, washed with brine (20 ml), then dried (MgSO₄), filtered and evaporated to give 2.06 g of 1-(tert-butyl-dimethyl-silanyloxy)-6-methyl-heptane-3,4-dione as a dark green liquid.

### 28D. Synthesis of N-[3-{4-[2-(tert-butyl-dimethyl-silanyloxv)-ethyl]-5-isobutyl-1H-imidazol-2-yl}-1-(tetrahydro-pyran-2-yl)-1H-pyrazol-4-yl]-2,6-difluoro-benzamide

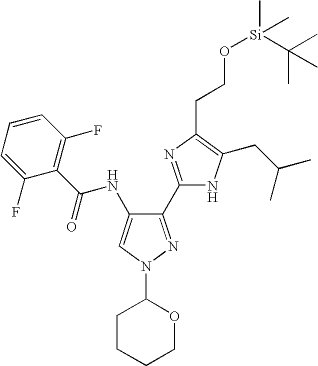

A mixture of 2,6-difluoro-N-[3-formyl-1-(tetrahydro-pyran-2-yl)-1H-pyrazol-4-yl]-benzamide (210 mg) and tert-butyl-dimethyl-(6-methyl-hept-3-ynyloxy)-silane (420 mg) in 2M ammonia in methanol (10 ml) was stirred at room temperature for 48 hours then evaporated. The crude product was purified by flash column chromatography, using 1:4 then 1:3 ethyl acetate/hexane. Evaporation of product containing fractions gave 240 mg of N-[3-{4-[2-(tert-butyl-dimethyl-silanyloxy)-ethyl]-5-isobutyl-1H-imidazol-2-yl}-1-(tetrahydro-pyran-2-yl)-1H-pyrazol-4-yl]-2,6-difluoro-benzamide. (LC/MS: Rₜ 3.76, [M+H]⁺ 588).

### 28E. Synthesis of 2,6-difluoro-N-{3-[4-(2-hydroxy-ethyl)-5-isobutyl-1H-imidazol-2-yl]-1H-pyrazol-4-yl}-benzamide

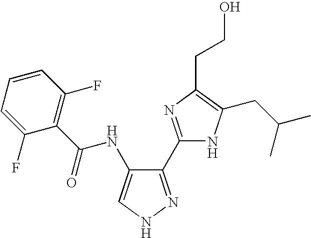

A solution of N-[3-{4-[2-(tert-butyl-dimethyl-silanyloxy)-ethyl]-5-isobutyl-1H-imidazol-2-yl}-1-(tetrahydro-pyran-2-yl)-1H-pyrazol-4-yl]-2,6-difluoro-benzamide (235 mg; 0.4 mmol) and 0.48 ml of tetrabutylammonium fluoride (1M solution in THF) dissolved in THF (5 ml) was stirred at room temperature overnight then diluted with ethyl acetate (20 ml) and washed with 2M hydrochloric acid (20 ml). The ethyl acetate layer was separated, washed with saturated sodium hydrogen carbonate (20 ml), dried (MgSO₄), filtered and evaporated. The crude material was dissolved in a mixture of concentrated hydrochloric acid/water/dioxan (1 ml:2 ml:2 ml) and heated at 120° C. (50W) for 10 mlnutes in a CEM discover microwave synthesiser. The reaction mixture was evaporated and the residue was partitioned between ethyl acetate (20 ml) and saturated sodium hydrogen carbonate (20 ml). The ethyl acetate layer was separated then dried (MgSO₄), filtered and evaporated. The crude product was purified by flash column chromatography, eluting with 2:1 then 4:1 ethyl acetate/hexane then ethyl acetate. Evaporation of product containing fractions gave 18 mg of 2,6-difluoro-N-{3-[4-(2-hydroxy-ethyl)-5-isobutyl-1H-imidazol-2-yl]-1H-pyrazol-4-yl}-benzamide as an off white solid. (LC/MS: Rₜ 1.93, [M+H]⁺ 390).

### Example 29

### Synthesis of 2,6-difluoro-N-{3-[5-isobutyl-4-(2-methanesulfonylamino-ethyl)-1H-imidazol-2-yl]-1H-pyrazol-4-yl}-benzamide.

### 29A. Synthesis of N-[3-{4-[2-(tert-butoxycarbonyl-methanesulfonyl-amino)-ethyl-5-isobutyl-1H-imidazol-2-yl}-1-(tetrahydro-pyran-2-yl)-1H-pyrazol-4-yl]-2,6-difluoro-benzamide

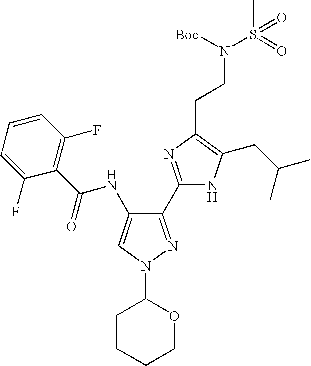

A solution of 2,6-difluoro-N-[3-[4-(2-hydroxy-ethyl)-5-isobutyl-1H-imidazol-2-yl]-1-(tetrahydro-pyran-2-yl)-1H-pyrazol-4-yl]-benzamide (100 mg; 0.2 mmol), N-Boc-methanesulphonamide (85 mg; 0.4 mmol), triphenylphosphine (110 mg; 0.4 mmol) and diethylazodicarboxylate (70 μl; 0.4 mmol) in THF (10 ml) was stirred at room temperature overnight then evaporated. The crude product was purified by flash column chromatography, eluting with 1:2 then 1:1 ethyl acetate. Evaporation of product containing fractions gave 60 mg of N-[3-{4-[2-(tert-butoxycarbonyl-metha nesulfonyl-amino)-ethyl]-5-isobutyl-1H-imidazol-2-yl}-1-(tetrahydro-pyran-2-yl)-1H-pyrazol-4-yl]-2,6-difluoro-benzamide.

### 29B. Synthesis of 2,6-difluoro-N-{3-[5-isobutyl-4-(2-methanesulfonplamino-ethyl)-1H-imidazol-2-yl]-1H-pyrazol-4-yl}-benzamide

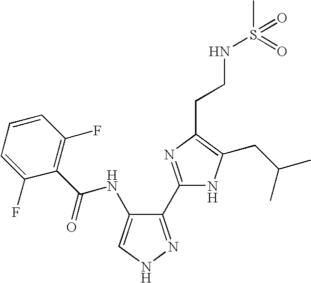

N-[3-{4-[2-(tert-butoxycarbonyl-methanesulfonyl-amino)-ethyl]-5-isobutyl-1H-imidazol-2-yl}-1-(tetrahydro-pyran-2-yl)-1H-pyrazol-4-yl]-2,6-difluoro-benzamide was dissolved in a mixture of concentrated hydrochloric acid/water/dioxan (1 ml:2 ml:2 ml) and heated at 120° C. (50W) for 10 mlnutes in a CEM discover microwave synthesiser then evaporated. The crude product was purified by preparative LC/MS, giving 10 mg of the title compound as grey solid. (LC/MS: Rₜ 1.94, [M+H]⁺ 467).

### Example 30

### Synthesis of 2,6-difluoro-N-{3-[5-(4-fluoro-phenyl)-4-morpholino-4-ylmeth-1H-imidazol-2-yl]-1H-pyrazol-4-yl}-benzamide

### 30A. Synthesis of 1-(3,3-diethoxy-prop-1-ynyl)-4-fluoro-benzene

To a solution of propargylaldehyde diethyl acetal (1 g, 7.81 mmoles), copper (I) iodide (6 mg, 0.03 mmoles), diethylamine (6.47 ml, 62.48 mmoles) and tetrakis (triphenylphosphine) palladium (0) (90 mg, 0.08 mmoles) in THF (20 ml) under nitrogen was added a dropwise solution of 4-fluoro-iodobenzene in THF (20 ml). The reaction mixture was left at ambient temperature for 24 hours. The reaction mixture was partitioned between EtOAc and HCl (2N). The organic portion was dried (MgSO₄), filtered, evaporated in vacuo, and then purified by flash column chromatography [Biotage SP4, 40S, flow rate 25 ml/min, gradient 100% Petrol to 1:2 EtOAc/Petrol] to give 1-(3,3-diethoxy-prop-1-ynyl)-4-fluoro-benzene as a colourless oil (900 mg, 52%).

### 30B. Synthesis of 3,3-diethoxy-1-(4-fluoro-phenyl)-propane-1,2-dione

To a solution of 1-(3,3-diethoxy-prop-1-ynyl)-4-fluoro-benzene (900 mg, 4.05 mmoles) in acetonitrile (25 ml) and CCl₄ (25 ml) was added a solution of ruthenium trichloride (25 mg, 0.12 mmoles) and sodium periodate (3.47 g, 16.20 mmoles) in water (36 ml). The resultant suspension was stirred vigorously for 1 hour. The reaction mixture was partitioned between EtOAc and water. The organic portion was dried (MgSO₄), filtered and the solvent removed in vacuo to give 3,3-diethoxy-1-(4-fluoro-phenyl)-propane-1,2-dione as a green oil (800 mg, 78%).

### 30C. Synthesis of N-[3-[4-diethoxymethyl-5-(4-fluoro-phenyl)-1H-imidazol-2-yl]-1-(4-methoxy-benzyl)-1H-pyrazol-4-yl]-2,6-difluoro-benzamide

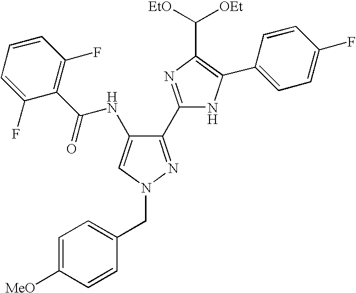

A sample of 3,3-diethoxy-1-(4-fluoro-phenyl)-propane-1,2-dione (110 mg, 0.431 mmoles) and 2,6-difluoro-N-[3-formyl-1-(4-methoxy-benzyl)-1H-pyrazol-4-yl]-benzamide (100 mg, 0.270 mmoles) was dissolved in methanolic ammonia (2N, 10 ml) and stirred at ambient temperature for 1 hour. The solvent was evaporated in vacuo. The residue was purified by flash column chromatography [Biotage SP4, 25S, flow rate 15 ml/min, gradient 3:7 EtOAc/Petrol to 7:3 EtOAc/Petrol] to give N-[3-[4-diethoxymethyl-5-(4-fluoro-phenyl)-1H-imidazol-2-yl]-1-(4-methoxy-benzyl)-1H-pyrazol-4-yl]-2,6-difluoro-benzamide as a colourless oil (105 mg, 64%). (LC/MS: Rₜ 4.04, [M+H]⁺ 606.10).

### 30D. Synthesis of 2,6-difluoro-N-[3-[5-(4-fluoro-phenyl)-4-formyl-1H-imidazol-2-yl]-1-(4-methoxy-benzyl)-1H-pyrazol-4-yl]-benzamide.

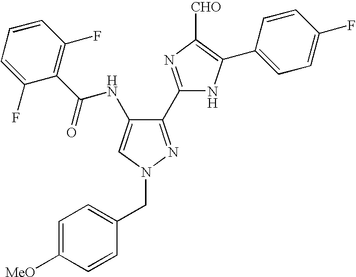

To a solution of N-[3-[4-diethoxymethyl-5-(4-fluoro-phenyl)-1H-imidazol-2-yl]-1-(4-methoxy-benzyl)-1H-pyrazol-4-yl]-2,6-difluoro-benzamide (105 mg, 0.174 mmoles) in acetone (10 ml) was added toluene sulphonic acid (165 mg, 0.868 mmoles). The solution was stirred at ambient temperature for 1 hour. The reaction mixture was partitioned between EtOAc and NaOH solution (2N). The organic portion was dried (MgSO₄), filtered, and evaporated in vacuo to give 2,6-difluoro-N-[3-[5-(4-fluoro-phenyl)-4-formyl-1H-imidazol-2-yl]-1-(4-methoxy-benzyl)-1H-pyrazol-4-yl]-benzamide as a white solid (85 mg, 92%). (LC/MS: Rₜ 3.55, [M+H]⁺ 532.06).

### 30E. Synthesis of 2,6-difluoro-N-[3-{5-(4-fluoro-phenyl-4-mormholin-4-ylmethyl-1H-imidazol-2-yl]-1-(4-methoxy-benzyl)-1H-pyrazol-4-yl]-benzamide.

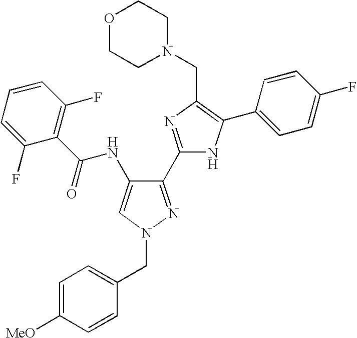

A solution of 2,6-difluoro-N-[3-[5-(4-fluoro-phenyl)-4-formyl-1H-imidazol-2-yl]-1-(4-methoxy-benzyl)-1H-pyrazol-4-yl]-benzamide (85 mg, 0.160 mmoles), sodium triacetoxy borohydride (41 mg, 0.192 mmoles), acetic acid (11 μl, 0.192 mmoles) and morpholine (17 μl, 0.192 mmoles) in dichloromethane (10 ml) was stirred at ambient temperature for 1 hour. Further sodium triacetoxy borohydride (80 mg, 0.377 mmoles), morpholine (35 μl, 0.395 mmoles) and acetic acid (22 μl, 0.384 mmoles) was added to the reaction mixture and then stirred at ambient temperature for a further 24 hours. The reaction mixture was partitioned between EtOAc and a saturated aqueous NaHCO₃ solution. The organic portion was dried (MgSO₄), filtered, evaporated in vacuo, and purified by flash column chromatography [Biotage SP4, 25S, flow rate 15 ml/min, gradient 2:3 EtOAc/Petrol to EtOAc] to give 2,6-difluoro-N-[3-[5-(4-fluoro-phenyl)-4-morpholin-4-ylmethyl-1H-imidazol-2-yl]-1-(4-methoxy-benzyl)-1H-pyrazol-4-yl]-benzamide as a colourless oil (45 mg, 47%). (LC/MS: Rₜ 2.43, [M+H]⁺ 603.10).

### 30F. Synthesis of 2,6-difluoro-N-{3-[5-(4-fluoro-iphenyl)-4-morpholino-4-ylmethyl-1H-imidazol-2-yl]-1H-pyrazol-4-yl}-benzamide

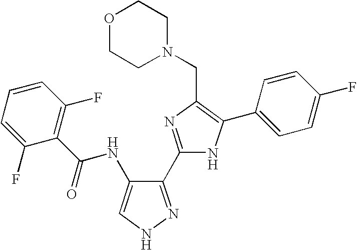

A solution of 2,6-difluoro-N-[3-[5-(4-fluoro-phenyl)-4-morpholin-4-ylmethyl-1H-imidazol-2-yl]-1-(4-methoxy-benzyl)-1H-pyrazol-4-yl]-benzamide (40 mg, 0.066 mmoles) and anisole (15 μl, 0.133 moles) in trifluoroacetic acid (2 ml) was heated at 130° C. (100W) in a CEM discover microwave synthesizer for ten minutes. Toluene (10 ml) was added and the reaction was evaporated in vacuo. The residue was purified using Biotage SP4 [12M, flow rate 5 ml/min, gradient 2:3 EtOAc/Petrol to EtOAc] and then by trituration with ether to give 2,6-difluoro-N-{3-[5-(4-fluoro-phenyl)-4-morpholino-4-ylmethyl-1H-imidazol-2-yl]-1H-pyrazol-4-yl}-benzamide as a pale yellow solid (7 mg, 22%). (LC/MS: Rₜ 2.04, [M+H]⁺ 483.07).

### Example 31

### Synthesis of 2,6-difluoro-N-(3-{5-(4-fluoro-phenyl)-4-[2-methoxm-ethylamino)-methyl]-1H-imidazol-2-yl}-1H-pyrazol-4-yl)-benzamide.

### 31A. Synthesis of N-[3-[4-diethoxymethyl-5-(4-fluoro-phenyl)-1H-imidazol-2-yl]-1-(tetrahydro-pyran-2-yl)-1H-pyrazol-4-yl]-2,6-difluoro-benzamide.

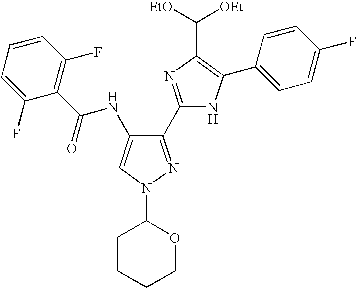

A solution of 2,6-difluoro-N-[3-formyl-1-(tetrahydro-pyran-2-yl)-1H-pyrazol-4-yl]-benzamide (528 mg, 1.57 mmoles) and 3,3-diethoxy-1-(4-fluoro-phenyl)-propane-1,2-dione (600 mg, 2.36 mmoles) in methanolic ammonia (2N, 20 ml) was stirred at ambient temperature for 24 hours. The solvent was removed in vacuo. The residue was purified [Biotage SP4 25S, flow rate 15 ml/min, gradient 1:3 EtOAc/Petrol to 3:2 EtOAc/Petrol] to give N-[3-[4-diethoxymethyl-5-(4-fluoro-phenyl)-1H-imidazol-2-yl]-1-(tetrahydro-pyran-2-yl)-1H-pyrazol-4-yl]-2,6-difluoro-benzamide as a colourless oil (700 mg, 78%). (LC/MS: Rₜ 3.84, [M+H]⁺ 570.08).

### 31B. Synthesis of 2,6-difluoro-N-[3-[5-(4-fluoro-phenyl)-4-formyl-1H-imidazol-2-yl]-1-(tetrahydro-pyran-2-yl)-1H-prazol-4-yl]-benzamide.

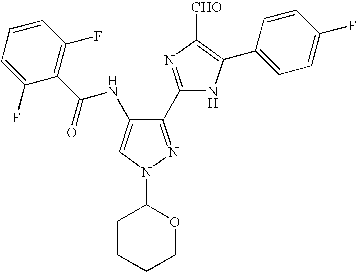

To a solution of N-[3-[4-diethoxymethyl-5-(4-fluoro-phenyl)-1H-imidazol-2-yl]-1-(tetrahydro-pyran-2-yl)-1H-pyrazol-4-yl]-2,6-difluoro-benzamide (700 mg, 1.23 mmoles) in acetone (10 ml) was added p-toluene sulphonic acid (23 mg, 0.12 mmoles). The reaction mixture was stirred at ambient temperature for 1 hour and then partitioned between EtOAc and NaOH solution (2N). The organic portion was dried (MgSO₄), filtered and evaporated in vacuo to give 2,6-difluoro-N-[3-[5-(4-fluoro-phenyl)-4-formyl-1H-imidazol-2-yl]-1-(tetrahydro-pyran-2-yl)-1H-pyrazol-4-yl]-benzamide as a pale yellow foam (600 mg, 99%). (LC/MS: Rₜ 3.40, [M+H]⁺ 496.06).

### 31 C. Synthesis of 2,6-difluoro-N-[3-{5-(4-fluoro-phenyl)-4-[(2-methoxy-ethylamino)-methyl]-1H-imidazol-2-yl}-1-(tetrahydro-pyran-2-yl)-1H-pyrazol-4-yl]-benzamide.

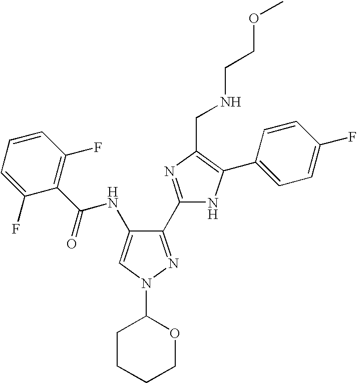

A solution of 2,6-difluoro-N-[3-[5-(4-fluoro-phenyl)-4-formyl-1H-imidazol-2-yl]-1-(tetrahydro-pyran-2-yl)-1H-pyrazol-4-yl]-benzamide (90 mg, 0.182 mmoles), sodium triacetoxy borohydride (46 mg, 0.218 mmoles), acetic acid (13 μl, 0.218 mmoles) and 2-methoxyethylamine (19 μl, 0.218 mmoles) in dichloromethane (5 ml) was stirred at ambient temperature for 24 hours. The reaction was partitioned between EtOAc and a saturated solution of NaHCO₃. The organic portion was dried (MgSO₄) and evaporated in vacuo to give 2,6-difluoro-N-[3-{5-(4-fluoro-phenyl)-4-[(2-methoxy-ethylamino)-methyl]-1H-imidazol-2-yl}-1-(tetrahydro-pyran-2-yl)-1H-pyrazol-4-yl]-benzamide as a colourless oil (80 mg, 79%). (LC/MS: Rₜ 2.37, [M+H]⁺ 555.07).

### 31D. Synthesis of 2,6-difluoro-N-(3-{5-(4-fluoro-phenyl)-4-[2-methoxy-ethylamino)-methyl]-1H-imidazol-2-yl}-1H-prazol-4-yl)-benzamide

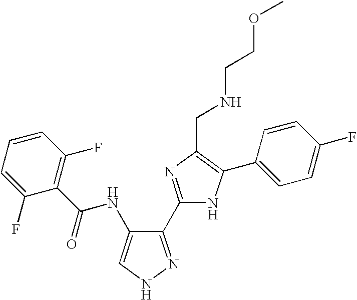

A solution of 2,6-difluoro-N-[3-{5-(4-fluoro-phenyl)-4-[(2-methoxy-ethylamino)-methyl]-1H-imidazol-2-yl}-1-(tetrahydro-pyran-2-yl)-1H-pyrazol-4-yl]-benzamide (80 mg, 0.144 mmoles) and anisole (31 μl, 0.289 mmoles) in trifluoroacetic acid (2 ml) was heated at 120° C. (100W) in a CEM discover microwave synthesiser for 10 minutes. Toluene (10 ml) was added, and the solvent evaporated in vacuo. The residue was triturated with ether, filtered and 2,6-difluoro-N-(3-{5-(4-fluoro-phenyl)-4-[2-methoxy-ethylamino)-methyl]-1H-imidazol-2-yl}-1H-pyrazol-4-yl)-benzamide was collected as a light brown solid (25nmg, 37%). (LC/MS: Rₜ 2.12, [M+H]⁺ 471.05).

| | | | Differences to | |
|---|---|---|---|---|
| Example | | | General | |
| Number. | Structure | Method used | Procedure? | LCMS |
| 32 | 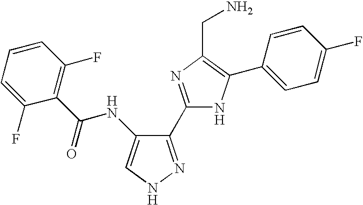 | Example 31 | 4-methoxybenzyl- amine in step 31C and 3 eqs of anisole in step 31D | [M + H]⁺ = 413 Rₜ = 2.08 |
| 33 | 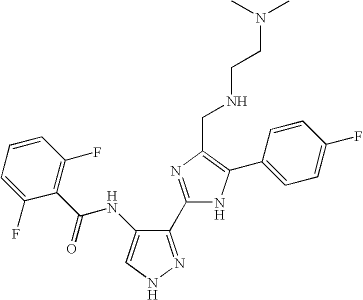 | Example 31 | N,N- dimethylethylene diamine in step 31C | [M + H]⁺ = 484 Rₜ = 1.86 |
| 34 | 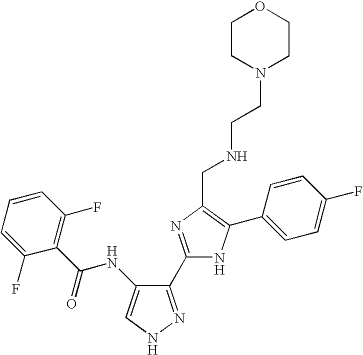 | Example 31 | 4-(2- aminoethyl)morpholine in step 31C | [M + H]⁺ = 526 Rₜ = 1.97 |

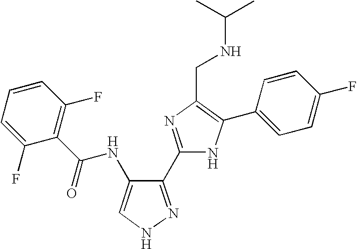

A solution of N-{3-[-4-aminomethyl-5-(4-fluoro-phenyl)-1H-imidazol]-1H-pyrazol-4-yl}-2,6-difluoro-benzamide (15 mg, 0.036 mmoles), acetone (3 μl, 0.040 mmoles), acetic acid (2 μl, 0.040 mmoles) and sodium triacetoxyborohydride (8 mg, 0.040 mmoles) in dichloromethane (10 ml) was stirred at ambient temperature for 2 hours. Further sodium triacetoxyborohydride (20 mg, 0.094 mmoles), acetic acid (6 μl, 0.095 mmoles) and acetone (101 μl, 0.136 mmoles) was added and then the reaction mixture stirred at ambient temperature for a further 24 hours. The reaction mixture was partitioned between EtOAc and water. The organic portion was dried (MgSO₄), filtered and evaporated in vacuo. The residue was triturated with ether and filtered to give a white solid. The solid was purified by preparative HPLC to give 2,6-difluoro-N-{3-[5-(4-fluoro-phenyl)-4-(isopropylamino-methyl)-1H-imidazol-2-yl}-1H-pyrazol-4-yl}-benzamide as a white solid (5 mg, 31%). (LC/MS: Rₜ 2.17, [M+H]⁺ 455.04).

### Example 36

### Synthesis of N-{3-(5-tert-butyl-4-methyl-1H-imidazol-2-yl)-1H-pyrazol-4-yl]-2,6-difluoro-benzamide.

### 36A. Synthesis of 4,4-dimethl-pentane-2,3-dione

To a solution of 4,4-dimethyl-pent-2-yne (2.5 g, 26 mmoles) in CCl₄ (25 ml) and acetonitrile (25 ml) in an ice-water bath was added a solution of ruthenium trichloride (162 mg) and sodium periodate (22.2 g, 104 mmoles). The suspension was allowed to warm to ambient temperature and stirred for 24 hours. The reaction mixture was partitioned between EtOAc and water. The organic portion was dried (MgSO₄), filtered and evaporated in vacuo to give 4,4-dimethyl-pentane-2,3-dione (2.1 g, 63%).

### 36B. Synthesis of N-[3-(5-tert-butyl-4-methyl-1H-imidazol-2-yl)-1-(tetrahydropyran-2-yl)-1H-pyrazol-4-yl]-2,6-difluoro-benzamide

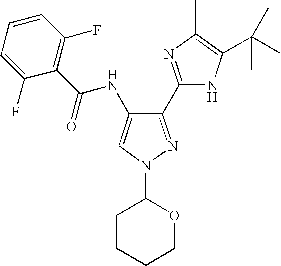

A solution of 4,4-dimethyl-pentane-2,3-dione (77 mg, 0.597 mmoles) and 2,6-difluoro-N-[3-formyl-1-(tetrahydro-pyran-2-yl)-1H-pyrazol-4-yl]-benzamnide (100 mg, 0.299 mmoles) in methanolic ammonia (2N, 10 ml) was stirred at ambient temperature for 1 hour. 4,4-Dimethyl-pentane-2,3-dione (70 mg, 0.55 mmoles) was added to the reaction mixture and stirred for a further hour. The solvent removed in vacuo. The residue was purified [Biotage SP4, 25S, flow rate 15 ml/min, gradient 1:4 EtOAc/Petrol to 3:2 EtOAc/Petrol] to give N-[3-(5-tert-butyl-4-methyl-1H-imidazol-2-yl)-1-(tetrahydropyran-2-yl)-1H-pyrazol-4-yl]-2,6-difluoro-benzamide as a colourless oil (110 mg, 83%). (LC/MS: Rₜ 2.34, [M+H]⁺ 444.13).

### 36C. Synthesis of N-[3-(5-tert- butyl-4-methyl-1H-imidazol-2-yl)-1H-pyrazol-4-yl]-2,6-difluoro-benzamide.

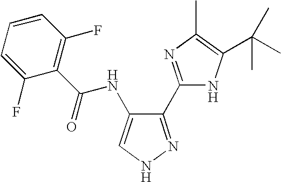

To a solution of N-[3-(5-tert-butyl-4-methyl-1H-imidazol-2-yl)-1-(tetrahydropyran-2-yl)-1H-pyrazol-4-yl]-2,6-difluoro-benzamide (110 mg, 0.248 mmoles) in ethanol (10 ml) was added p-toluene sulphonic acid (95 mg, 0.496 mmoles). The solution was heated at 70° C. for 1 hour, partitioned between EtOAc and NaOH solution (2N). The organic portion was dried (MgSO₄), filtered and evaporated in vacuo to give N-[3-(5-tert-butyl-4-methyl-1H-imidazol-2-yl)-1H-pyrazol-4-yl]-2,6-difluoro-benzamide as a white solid (80 mg, 90%). (LC/MS: Rₜ 2.08, [M+H]⁺ 360.08).

### Example 37

### Synthesis of N-[3-(4,5-dimethyl-1H-imidazol-2-yl)-1H-pyrazol-4-yl]-2,6-difluoro-benzamide.

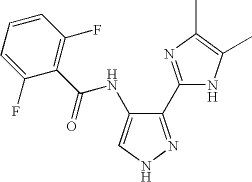

The compound was made in a manner analogous to Example 36, but using 2,3-butadione in step B in place of 4,4-dimethyl-pentane-2,3-dione to give N-[3-(4,5-dimethyl-1H-imidazol-2-yl)-1H-pyrazol-4-yl]-2,6-difluoro-benzamide as a white solid (35 mg). (LC/MS: Rₜ 1.62, [M+H]⁺ 318.02).

### Example 38

### Synthesis of 2,6-difluoro-N-{3-[4-(morpholine-4-carbonyl)-5-trifluoromethyl-1H-imidazol-2-yl]-1H-pyrazol-4-yl}-benzamide.

### 38A. Synthesis of 4,4,4-trifluoro-2-hydroxyimino-3-oxo-butyric acid ethyl ester

To a solution of ethyl-4,4,4-trifluoroacetoacetate (4.6 g, 25 mmoles) in acetic acid (12 ml) in an ice-water bath was added dropwise a solution of sodium nitrite (4 g, 57.97 mmoles) in water (14 ml) over 1 hour. The reaction mixture was stirred for a fher 2 hours at 15° C. Toluene (20 ml) was added and the reaction mixture evaporated in vacuo. The residue was partitioned between EtOAc and a saturated solution of NaHCO₃. The organic portion was washed with a saturated solution of NaHCO₃, brine, dried (MgSO₄), filtered and evaporated in vacuo to give 4,4,4-trifluoro-2-hydroxyimino-3-oxo-butyric acid ethyl ester as a white solid (3.8 g, 71%).

### 38B. Synthesis of 2-[4-(2,6-difluoro-benzoy1amino)-1-(tetrahydro-pyran-2-y1)-1H-pyrazol-3-yl]-1-hydroxy-5-trifluoromethyl-1H-imidazole-4-carboxylic acid ethyl ester.

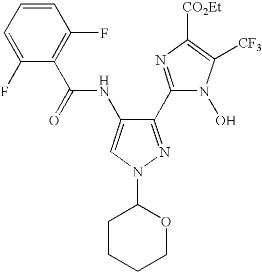

A solution of 4,4,4-trifluoro-2-hydroxyimino-3-oxo-butyric acid ethyl ester (1.3 g, 5.97 mmoles) and 2,6-difluoro-N-[3-formyl-1-(tetrahydro-pyran-2-yl)-1H-pyrazol-4-yl]-benzamide (1 g, 2.99 mmoles) in methanolic ammonia (2N, 20 ml) was stirred at ambient temperature for 24 hours. The solvent was removed in vacuo. The residue was purified [Biotage SP4, 40S, flow rate 35 ml/min, gradient 1:4 EtOAc/Petrol to 4:1 EtOAc/Petrol] to give 2-[4-(2,6-difluoro-benzoylamino)-1-(tetrahydro-pyran-2-yl)-1H-pyrazol-3-yl]-1-hydroxy-5-trifluoromethyl-1H-imidazole-4-carboxylic acid ethyl ester as a yellow solid (0.45 g, 29%). (LC/MS: Rₜ 3.42, [M+H]⁺ 530.07).

### 38B. Synthesis of 2,6-difluoro-N-[3-[1-hydroxy-4-morpholine-4-carbonyl-5-trifluoromethyl-1H-imidazol-2-yl]-1-(tetrahydro-pyran-2-yl)-1H-]2razol-4-yl]-benzamide

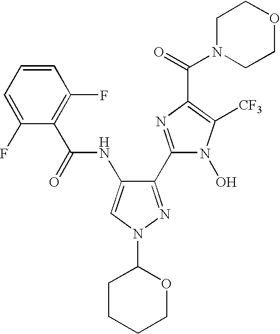

To a solution of 2-[4-(2,6-difluoro-benzoylamino)-1-(tetrahydro-pyran-2-yl)-1H-pyrazol-3-yl]-1-hydroxy-5-trifluoromethyl-1H-imidazole-4-carboxylic acid ethyl ester (450 mg, 0.874 mmoles) in methanol (10 ml) was added a solution of NaOH (2N, 10 ml). The reaction mixture was stirred at ambient temperature for 3 hours, diluted with EtOAc, then washed with a 5% citric acid solution, dried (MgSO₄), filtered and the solvent removed in vacuo to give a yellow solid. To a solution of the solid in dichloromethane (10 ml) was added EDC (115 mg, 0.60 mmoles), HOBt (81 mg, 0.60 mmoles) and morpholine (52 μl, 0.60 mmoles). The reaction mixture was stirred at ambient temperature for 24 hours. The reaction mixture was partitioned between EtOAc and a saturated solution of NaHCO₃. The organic portion was dried (MgSO₄), filtered and evaporated in vacuo. The residue was purified [Biotage SP4, 25M, flow rate 30 ml/min, gradient EtOAc to 1:4 MeOH/EtOAc] to give 2,6-difluoro-N-[3-[1-hydroxy-4-morpholine-4-carbonyl)-5-trifluoromethyl-1H-imidazol-2-yl]-1-(tetrahydro-pyran-2-yl)-1H-pyrazol-4-yl]-benzamide as a white solid (200 mg, 70%). (LC/MS: Rₜ 3.03, [M+H]⁺ 571.07).

### 38D. Synthesis of 2,6-difluoro-N-{3-[4-(morpholine-4-carbonyl)-5-trifluoromethyl-1H-imidazol-2-yl]-1H-pyrazol-4-yl}-benzamide

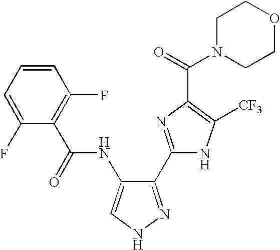

A solution of 2,6-difluoro-N-[3-[1-hydroxy-4-morpholine-4-carbonyl)-5-trifluoromethyl-1H-imidazol-2-yl]-1-(tetrahydro-pyran-2-yl)-1H-pyrazol-4-yl]-benzamide (200 mg, 0.351 mmoles) and anisole (76 μl, 0.702 mmoles) in trifluoroacetic acid (2 ml) was heated at 100° C. (80W) in a CEM discover microwave synthesizer. Toluene (10 ml) was added to the reaction mixture, which was then evaporated in vacuo. The residue was triturated with ether and the white solid was collected by filtration. A solid precipitated out of the filtrate, which was filtered and combined with the other white solid. The solid (120 mg, 0.25 mmoles) was dissolved in ethanol (10 ml). To this solution was added p-toluene sulphonic acid (95 mg, 0.50 mmoles) and palladium on carbon (10%, 6 mg) under nitrogen. The solution was hydrogenated at STP for 48 hours. The suspension was filtered through celite, and the filtrate partitioned between EtOAc and a saturated solution of NaHCO₃. The organic portion was dried (MgSO₄), filtered and evaporated in vacuo to give 2,6-difluoro-N-{3-[4-(morpholine-4-carbonyl)-5-trifluoromethyl-1H-imidazol-2-yl]-1H-pyrazol-4-yl}-benzamide as a white solid (30 mg, 20%). (LC/MS: Rₜ 2.68, [M+H]⁺ 471.03).

### Example 39

### Synthesis of 2,6-difluoro-N-{3-[4-hydroxymethyl-5-(4-morpholin-4-yl-phenvyl)-1H-imidazol-2-yl]-1H-pyrazol-4-yl}-benzamide.

### 39A. Synthesis of 2,6-difluoro-N-{3-[4-formyl-5-(4-morpholin-4-yl-phenyl)-1H-imidazol-2-yl]-1H-prazol-4-yl}-benzamide

To a solution of 3,3-diethoxy-1-(4-fluoro-phenyl)-propane-1,2-dione (4.5 g, 17.72 mmoles, example 30B) in DMSO (30 ml) was added morpholine (1.9 ml, 21.26 mmoles) and potassiumn carbonate (4.9 g, 35.44 mmoles). The suspension was heated at 90° C. for 2 hours. The reaction mixture was partitioned between EtOAc and water. The organic portion was dried (MgSO₄), filtered and evaporated in vacuo to give 3,3-diethoxy-1-(4-morpholin-4-yl-phenyl)-propane-1,2-dione as a bright yellow oil (400 mg, 7%).

### 39B. Synthesis of N-[3-[4-diethoxymethyl-5-(4-morpholin-4-yl-phenyl)-1H-imidazol-2-yl]-1-(tetrahydro-pyran-2-yl-1H-pyrazol-4-yl]-2,6-difluoro-benzamide

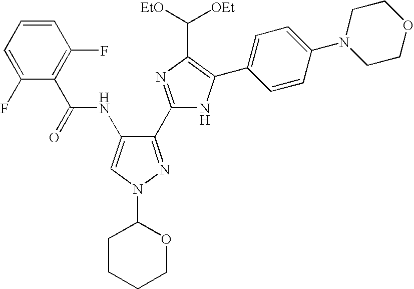

A solution of 3,3-diethoxy-1-(4-morpholin-4-yl-phenyl)-propane-1,2-dione (400 mg, 1.25 mmoles) and 2,6-difluoro-N-[3-formyl-1-(tetrahydro-pyran-2-yl)-1H-pyrazol-4-yl]-benzamide (348 mg, 1.04 mmoles) in methanolic ammonia (2N, 20 ml) was stirred at ambient temperature for 3 hours. The solvent was removed in vacuo. The residue was purified [Biotage SP4, 25M, flow rate 25 ml/min, gradient 1:4 EtOAc/Petrol to EtOAc] to give N-[3-[4-diethoxymethyl-5-(4-morpholin-4-yl-phenyl)-1H-imidazol-2-yl]-1-(tetrahydro-pyran-2-yl)-1H-pyrazol-4-yl]-2,6-difiuoro-benzamidc as a viscous yellow oil (170 mg, 26%).

### 39C. Synthesis of 2,6-difluoro-N-{3-[4-formyl-5-(4-morpholin-4-yl-phenly)-1H-imidazol-2-yl]-1H-pyrazol-4-yl}-benzamide

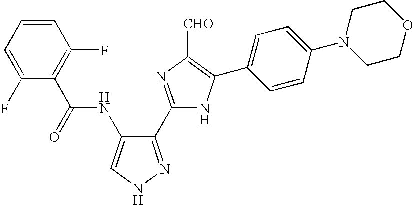

To a solution of N-[3-[4-diethoxymethyl-5-(4-morpholin-4-yl-phenyl)-1H-imidazol-2-yl]-1-(tetrahydro-pyran-2-yl)-1H-pyrazol-4-yl]-2,6-difluoro-benzamide (170 mg, 0.27 mmoles) in ethanol (10 ml) was added p-toluene sulphonic acid (103 mg, 0.54 mmoles) and the reaction mixture was heated at 70° C. for 1 hour. The reaction mixture was partitioned between EtOAc and a saturated solution of NaHCO₃. The organic portion was dried (MgSO₄), filtered and evaporated in vacuo. The residue was triturated with ether and filtered to give 2,6-difluoro-N-{3-[4-formyl-5-(4-morpholin-4-yl-phenyl)-1H-imidazol-2-yl]-1H-pyrazol-4-yl}-benzamide as a light brown solid (50 mg, 33%). (LC/MS: Rₜ 2.81, [M+H]⁺ 479.02).

### 39D. Synthesis of 2,6-difluoro-N-{3-[4-hydroxymethyl-5-(4-morpholin-4-yl-phenyl)-1H-imidazol-2-yl]-1H-pyrazol-4-yl}-benzamide

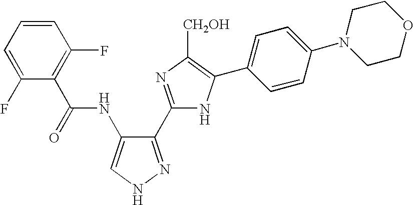

To a solution of 2,6-difluoro-N-{3-[4-formyl-5-(4-morpholin-4-yl-phenyl)-1H-imidazol-2-yl]-1H-pyrazol-4-yl}-benzamide (50 mg, 0.10 mmoles) in ethanol (10 ml) was added sodium borohydride (8 mg, 0.20 mmoles) and the reaction mixture stirred at ambient temperature for 1 hour. The reaction mixture was partitioned between EtOAc and a saturated solution of NaHCO₃. The organic portion was dried (MgSO₄) filtered and evaporated in vacuo. The residue was triturated with ether and filtered to give 2,6-difluoro-N-{3-[4-hydroxymethyl-5-(4-morpholin-4-yl-phenyl)-1H-imidazol-2-yl]-1H-pyrazol-4-yl}-benzamide as a white solid (15 mg, 31%). (LC/MS: Rₜ 2.17, [M+H]⁺ 481.06).

### Example 40

### Synthesis of 2,6-difluoro-N-[3-(4-methyl-5-trifluoromethyl-1H-imidazol-2-yl)-1H-pyrazol-4-yl]-benzamide.

### 40A Synthesis of 1,1,1,-trifluoro-butane-2,3-dione-3-oxime

To a solution of 1,1,1-trifluoro-2-butanone (5 g, 39.68 mmoles) in acetic acid (9.5 ml) cooled in an ice-water bath was added dropwise a solution of sodium nitrite (6.3 g, 91.27 mmoles) in water (11 ml) over 1 hour. The reaction mixture was stirred at ambient temperature for a further hour. Toluene (20 ml) was added and the solvent was removed in vacuo. The residue was partitioned between EtOAc and a saturated solution of NaHCO₃. The organic portion was dried (MgSO₄), filtered and evaporated in vacuo to give 1,1,1,-trifluoro-butane-2,3-dione-3-oxime as a colourless oil (1.0 g, 16%).

### 40B Synthesis of 2,6-difluoro-N-[3-(1-hdroxy-4-methyl-5-trifluoromethyl-1H-imidazol-2-yl)-1-(tetrahydro-pyran-2-yl)-1H-pyrazol-4-yl]-benzamide

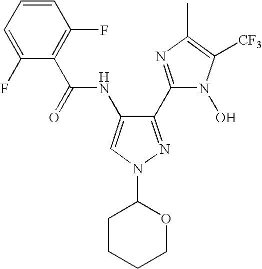

A solution of 1,1,1,-trifluoro-butane-2,3-dione-3-oxime (1.0 g, 6.45 mmoles) and 2,6-difluoro-N-[3-formyl-1-(tetrahydro-pyran-2-yl)-1H-pyrazol-4-yl]-benzamide (300 mg, 0.90 mmoles) in methanolic ammonia (2N, 10 ml) was stirred at ambient temperature for 24 hours. The solvent was removed in vacuo, the residue triturated with ether and filtered to give 2,6-difluoro-N-[3-(1-hydroxy-4-methyl-5-trifluoromethyl-1H-imidazol-2-yl)-1-(tetrahydro-pyran-2-yl)-1H-pyrazol-4-yl]-benzamide as a white solid (290 mg, 68%). (LC/MS: Rₜ 3.33, [M+H]⁺ 472.09).

### 40C. Synthesis of 2,6-difluoro-N-[3-(4-methyl-5-trifluoromethyl-1H-imidazol-2-yl)-1H-pyrazol-4-yl]-benzamide

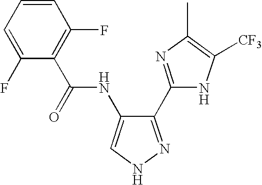

To a solution of 2,6-difluoro-N-[3-(1-hydroxy-4-methyl-5-trifluoromethyl-1H-imidazol-2-yl)-1-(tetrahydro-pyran-2-yl)-1H-pyrazol-4-yl]-benzamide (290 mg, 0.62 mmoles) in methanol (10 ml) in an ice-water bath was added dropwise a solution of TiCl₃ (3 ml, 15% solution in 20-30% HCl). The reaction mixture was stirred at ambient temperature for 2 hours and then partitioned between EtOAc and water. The organic portion was dried (MgSO₄), filtered and evaporated in vacuo. The residue was purified by using Biotage SP4 [25S, flow rate 25 ml/min, gradient 3:2 EtOAc/Petrol to EtOAc] and then triturated with ether:petrol (1:1) and filtered to give 2,6-difluoro-N-[3-(4-methyl-5-trifluoromethyl-1H-imidazol-2-yl)-1H-pyrazol-4-yl]-benzamide as a white solid (50 mg, 22%). (LC/MS: Rₜ 2.98, [M+H]⁺371.97).

### Example 41

### Synthesis of N-[3-(5-cyano-4-methyl-1H-imidazol-2-yl)-1H-pyirazol-4-yl]-2,6-difluoro-benzamide

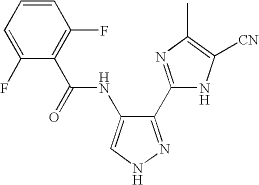

A solution of 2,6-difluoro-N-[3-(4-methyl-5-trifluoromethyl-1H-imidazol-2-yl)-1H-pyrazol-4-yl]-benzamide (10 mg, 0.03 mmoles), concentrated ammonium hydroxide (5 ml) and methanol (2 ml) was heated at 60° C. for 48 hours. The reaction mixture was partitioned between EtOAc and water. The aqueous layer was washed with EtOAc. The organic portions were combined, dried (MgSO₄), filtered and evaporated in vacuo to give N-[3-(5-cyano-4-methyl-1H-imidazol-2-yl)-1H-pyrazol-4-yl]-2,6-difluoro-benzamide as a white solid (9 mg, 91%). (LC/MS: Rₜ 2.59, [M+H]⁺ 329.01).

### Example 42

### Synthesis of 2-fluoro-6-methoxy-N-[3-(5-methyl-4-trifluoromethyl-1H-imidazol-2-yl)-1H-pyrazol-4-yl]-benzamide.

### 42A. Synthesis of 3,3-dibromo-1,1,1-trifluoro-butan-2-one

A suspension of 1,1,1-trifluoro-butan-2-one (10 g, 79.4 mmoles), N-bromosuccinimide (36.7 g, 206.3 mmoles) and benzoyl peroxide (25 mg, 0.1 mmoles) in carbon tetrachloride (100 ml) was irradiated at 70° C. with a tungsten lamp for 36 hours. The suspension was filtered and the filtrate was evaporated in vacuo. The residue was triturated with petrol, filtered and the filtrate was evaporated in vacuo to give 3,3-dibromo-1,1,1-trifluoro-butan-2-one as a pale yellow liquid (7.1 g, 32%).

### 42B. Synthesis of 4-benzyloxycarbonylamino-1-(tetrahydro-pyran-2-yl)-1H-pyrazole-3-carboxylic acid

To a solution of 4-amino-1-(tetrahydro-pyran-2-yl)-1H-pyrazole-3-carboxylic acid methyl ester (5.2 g, 23.11 mmoles) in dioxane (100 ml) was added a solution of sodium hydroxide (2N, 100 ml) and benzyl chloroformate (3.6 ml, 25.42 mmoles). The reaction mixture was stirred at ambient temperature for 24 hours. Further benzyl chloroformate (3.6 ml, 25.42 mmoles) was added and the reaction mixture stirred for a further 24 hours. The reaction mixture was partitioned between ether and water. The aqueous portion was acidified to pH 2 with a solution of HCl (2N) and partitioned against EtOAc. The organic portion was dried (MgSO₄), filtered and evaporated in vacuo to give 4-benzyloxycarbonylamino-1-(tetrahydro-pyran-2-yl)-1H-pyrazole-3-carboxylic acid as a white solid (7.8 g, 98%). (LC/MS: Rₜ 2.85, [M+H]⁺ 346.05).

### 42C. Synthesis of 4-benzyloxycarbonlamino-1-(tetrahydro-pyran-2-yl)-1H-pyrazole-3-carboxylic acid methl ester

To a stirred solution of 4-benzyloxycarbonylamino-1-(tetrahydro-pyran-2-yl)-1H-pyrazole-3-carboxylic acid (1.3 g, 3.77 mmoles) in methanol (10 ml) was added EDC (867 mg, 4.52 mmoles) and DMAP (46 mg, 0.38 mmoles). The reaction mixture was stirred at ambient temperature for 24 hours. The reaction mixture was diluted with EtOAc, washed with water, a solution of HCl (2N) and then a solution of sodium hydroxide (2N). The organic portion was dried (MgSO₄), filtered and evaporated in vacuo. The residue was purified by using Biotage SP4 (40S, flow rate 40 ml/min, gradient 3:7 EtOAc/Petrol to 3:2 EtOAc/Petrol) to give 4-benzyloxycarbonylamino-1-(tetrahydro-pyran-2-yl)-1H-pyrazole-3-carboxylic acid methyl ester as a colourless oil (1.35 g, 100%). (LC/MS: Rₜ 3.23, [M+H]⁺ 360.09).

### 42D. Synthesis of [3-hydroxymethyl-1-(tetrahydro-pyran-2-yl)-1H-pyrazol-4-yl]-carbamic acid benzyl ester

To a solution of 4-benzyloxycarbonylamino-1-(tetrahydro-pyran-2-yl)-1H-pyrazole-3-carboxylic acid methyl ester (1.35 g, 3.76 mmoles) in THF (50 ml) under nitrogen at −78° C. was added dropwise a solution of diisobutylaluminium hydride in THF (1M, 38 ml). The reaction mixture was warmed to 0° C. and a solution of saturated Na₂SO₄ added (50 ml).

The suspension formed was filtered and the filtrate partitioned between EtOAc and brine. The organic portion was dried (MgSO₄), filtered and evaporated in vacuo to give [3-hydroxymethyl-1-(tetrahydro-pyran-2-yl)-1H-pyrazol-4-yl]-carbamic acid benzyl ester as a colourless oil (1.2 g, 96%). (LC/MS: Rₜ 2.57, [M+H]⁺ 332.08).

### 42E. Synthesis of [3-formyl-1-(tetrahydro-pyran-2-yl)-1H-pyrazol-4-yl]-carbamic acid benzyl ester

To a solution of [3-hydroxymethyl-1-(tetrahydro-pyran-2-yl)-1H-pyrazol-4-yl]-carbamic acid benzyl ester (1.2 g, 3.6 mmoles) in acetone (20 ml) was added MnO₂ (3.2 g, 36.0 mmoles) and the resisitant suspension stirred at ambient temperature for 48 hours. Further MnO₂ (3.2 g, 36.0 mmoles) was added and the suspension stirred for a further 24 hours. The suspension was filtered through celite, and the filtrate was evaporated in vacuo. The residue was purified using Biotage SP4 (40M, flow rate 40 ml/min, gradient 1:9 EtOAc/Petrol to 3:2 EtOAc/Petrol) to give [3-formyl-1-(tetrahydro-pyran-2-yl)-1H-pyrazol-4-yl]-carbamic acid benzyl ester as a pale yellow solid (700 mg, 59%). (LC/MS: Rₜ 3.27, [M+H]⁺ 330.07).

### 42F. Synthesis of [3-(5-methyl-4-trifluoromethyl-1H-imidazol-2-yl)-1-(tetrahydro-pyran-2-yl)-1H-pyrazol-4-yl]carbamic acid benzyl ester

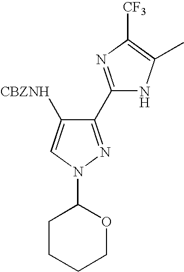

A solution of [3-formyl-1-(tetrahydro-pyran-2-yl)-1H-pyrazol-4-yl]-carbamic acid benzyl ester (700 mg, 2.13 mmoles) and a sample of 3,3-dibromo-1,1,1-trifluoro-butan-2-one (868 mg, 4.26 mmoles) in methanolic ammonia (2N, 20 ml) was stirred at ambient temperature for 48 hours. The solvent was evaporated in vacuo. The residue was purified by using Biotage SP4 (25M, flow rate 25 ml/min, gradient 1:9 EtOAc/Petrol to 3:2 EtOAc/Petrol) to give [3-(5-methyl-4-trifluoromethyl-1H-imidazol-2-yl)-1-(tetrahydro-pyran-2-yl)-1H-pyrazol-4-yl]carbamic acid benzyl ester as a pale yellow oil (450 mg, 47%). (LC/MS: Rₜ 3.62, [M+H]⁺ 450.20).

### 42G. Synthesis of 3-(5-methyl-4-trifluoromethyl-1H-imidazol-2-yl)-1-(tetrahydro-pyran-2-yl)-1H-pyrazol-4-ylamine

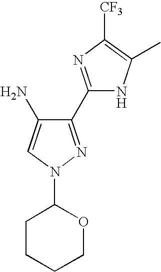

To a solution of give [3-(5-methyl-4-trifluoromethyl-1H-imidazol-2-yl)-1-(tetrahydro-pyran-2-yl)-1H-pyrazol-4-yl]carbamic acid benzyl ester (450 mg, 1.0 mmoles) in ethanol (10 ml) under nitrogen was added palladium on carbon (10% 45 mg). The suspension was hydrogenated at STP for 3 hours. The suspension was filtered through celite, and the filtrate evaporated in vacuo to give 3-(5-methyl-4-trifluoromethyl-1H-imidazol-2-yl)-1-(tetrahydro-pyran-2-yl)-1H-pyrazol-4-ylamine as a pale yellow solid (290 mg, 92%). (LC/MS: Rₜ 2.67, [M+H]⁺ 316.09).

### 42H. Synthesis of 2-fluoro-6-methoxy-N-methyl-N-[3-(5-methyl-4-trifluoromethyl-1H-imidazol-2-yl-1-(tetrahydro-pyran-2-yl)-1H-pyrazol-4-yl]benzamide

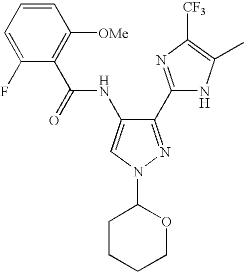

To a solution of 3-(5-methyl-4-trifluoromethyl-1H-imidazol-2-yl)-1-(tetrahydro-pyran-2-yl)-1H-pyrazol-4-ylamine (50 mg, 0.16 mmoles) in dichloromethane (5 ml) was added EDC (37 mg, 0.19 mmoles), HOBt (26 mg, 0.19 mmoles) and 2-fluoro-6-methoxybenzoic acid (27 mg, 0.16 mmoles). The reaction mixture was stirred at ambient temperature for 3 hours. Further EDC (20 mg, 0.10 mmoles), HOBt (15 mg, 0.11 moles) and 2-fluoro-6-methoxybenzoic acid (15 mg, 0.09 mmoles) were added, and the reaction mixture stirred at ambient temperature for a further 24 hours. The reaction mixture was purified by using Biotage SP4 (25S, flow rate 25 ml/min, gradient 1:9 EtOAc/Petrol to 1:1 EtOAc/Petrol) to give 2-fluoro-6-methoxy-N-methyl-N-[3-(5-methyl-4-trifluoromethyl-1H-imidazol-2-yl)-1-(tetrahydro-pyran-2-yl)-1H-pyrazol-4-yl]benzamide as a pale yellow oil (50 mg, 67%). (LC/MS: Rₜ 3.34, [M+H]⁺ 468.16).

### 42I. Sythesis of 2-fluoro-6-methoxy-N-[3-(5-methyl-4-trifluoromethyl-1H-imidazol-2-yl)-1H-pyazol-4-yl]-benzamide

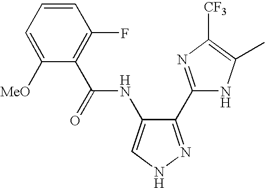

To a solution of 2-fluoro-6-methoxy-N-methyl-N-[3-(5-methyl-4-trifiuoromethyl-1H-imidazol-2-yl)-1-(tetrahydro-pyran-2-yl)-1H-pyrazol-4-yl]benzamide (50 mg, 0.11 moles) in ethanol (10 ml) was added toluene sulphonic acid (63 mg, 0.33 mmoles). The reaction mixture was heated at 70° C. for 24 hours. The reaction mixture was partitioned between EtOAc and a solution of sodium hydroxide (2N). The organic portion was dried (MgSO₄), filtered and evaporated in vacuo. The residue was triturated with ether: petrol (1:1) and filtered to give 2-fluoro-6-methoxy-N-[3-(5-methyl-4-trifluoromethyl-1H-imidazol-2-yl)-1H-pyrazol-4-yl]-benzamide as a white solid (10 mg, 24%). (LC/MS: Rₜ 2.96, [M+H]⁺ 384.07).

### Examples 43-44

By following the procedures set out in Example 42, modified as necessary, the compounds of Examples 43 and 44 were prepared.

| | | | Differences to | |
|---|---|---|---|---|
| Example | | Method | general | |
| number | Structure | Used | procedure | LC/MS |
| 43 | 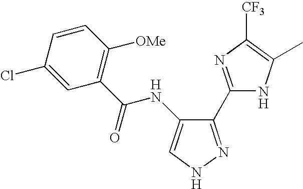 | Ex. 42 | 2-methoxy-5- chlorobenzoyl chloride and triethylamine instead of 2- fluoro-6 methoxybenoic acid, EDC and HOBt in step 42H | [M + H]⁺400.09 Rₜ 3.29 |
| 44 | 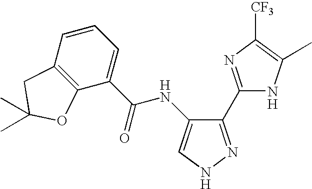 | Example 42 | 2,2-dimethyl- 2,3-dihydro- 1-benzofuran- 7-carboxylic acid used in step 42H instead of 2-fluoro-6- methoxybenzoic acid | [M + H]⁺406.10 Rₜ 3.23 |

### Example 45

### 3-Fluoro-5-(4-methyl-piperazin-1-yl)-N-[3-(4-methyl-5-trifluoromethyl-1H-imidazol-2-yl)-1H-pyrazol-4-yl]-benzamide

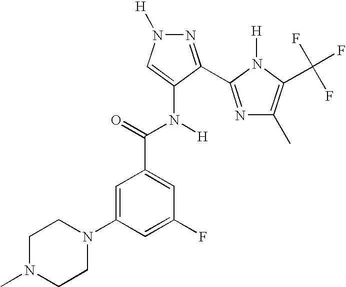

The title compound was prepared a method as described in the preceding examples.

### Biological Activity

### Example 46

Measurement of CDK2 Kinase Inhibitorv Activity (IC₅₀)

Compounds of the invention were tested for kinase inhibitory activity using either Protocol A or Protocol B.

Protocol A

1.7 μl of active CDK2/CyclinA (Upstate Biotechnology, 10 U/μl) is diluted in assay buffer (250 μl of 10 × strength assay buffer (200 mM MOPS pH 7.2, 250 mM β-glycerophosphate, 50 mM EDTA, 150 mM MgCl₂), 11.27 μl 10 mM ATP, 2.5 μl 1M DTT, 25 μl 100 mM sodium orthovanadate, 708.53 μl H₂O), and 10 μl mixed with 10 μl of histone substrate mix (60 μl bovine histone H1 (Upstate Biotechnology, 5 mg/ml), 940 μl H₂O, 35 μCi γ³³P-ATP) and added to 96 well plates along with 5 μl of various dilutions of the test compound in DMSO (up to 2.5%). The reaction is allowed to proceed for 5 hours before being stopped with an excess of ortho-phosphoric acid (30 μl at 2%).

γ³³P-ATP which remains unincorporated into the histone H1 is separated from phosphorylated histone H1 on a Millipore MAPH filter plate. The wells of the MAPH plate are wetted with 0.5% orthophosphoric acid, and then the results of the reaction are filtered with a Millipore vacuum filtration unit through the wells. Following filtration, the residue is washed twice with 200 μl of 0.5% orthophosphoric acid. Once the filters have dried, 25 μl of Microscint 20 scintillant is added, and then counted on a Packard Topeount for 30 seconds.

The % inhibition of the CDK2 activity is calculated and plotted in order to determine the concentration of test compound required to inhibit 50% of the CDK2 activity (IC₅₀).

The compounds of Examples 1 to 17 each have IC₅₀ values of less than 10 μM or provide at least 50% inhibition of the CDK2 activity at a concentration of 10 μM. Preferred compounds have IC₅₀ values of less than 1 μM.

Protocol B

Activated CDK2/CyclinA (Brown et al, Nat. Cell Biol., 1, pp438-443, 1999; Lowe, E. D., et al Biochemistry, 41, pp15625-15634, 2002) is diluted to 125 pM in 2.5× strength assay buffer (50 mM MOPS pH 7.2, 62.5 mM β-glycerophosphate, 12.5 mM EDTA, 37.5 mM MgC₂, 112.5 mM ATP, 2.5 mM DTT, 2.5 mM sodium orthovanadate, 0.25 mg/ml bovine serum albumin), and 10 μl mixed with 10 μl of histone substrate mix (60 μl bovine histone H1 (Upstate Biotechnology, 5 mg/ml), 940 μl H₂O, 35 μCi γ³³P-ATP) and added to 96 well plates along with 5 μl of various dilutions of the test compound in DMSO (up to 2.5%). The reaction is allowed to proceed for 2 to 4 hours before being stopped with an excess of ortho-phosphoric acid (5 μl at 2%).

γ³³P-ATP which remains unincorporated into the histone H1 is separated from phosphorylated histone H1 on a Millipore MAPH filter plate. The wells of the MAPH plate are wetted with 0.5% orthophosphoric acid, and then the results of the reaction are filtered with a Millipore vacuum filtration unit through the wells. Following filtration, the residue is washed twice with 200 μl of 0.5% orthophosphoric acid. Once the filters have dried, 20 μl of Microscint 20 scintillant is added, and then counted on a Packard Topcount for 30 seconds.

The % inhibition of the CDK2 activity is calculated and plotted in order to determine the concentration of test compound required to inhibit 50% of the CDK2 activity (IC₅₀).

CDK1/CyclinB Assay.

CDK1/CyclinB assay is identical to the CDK2/CyclinA above except that CDK1/CyclinB (Upstate Discovery) is used and the enzyme is diluted to 6.25 nM.

### Example 47

GSK3-B/Aurora Kinase Inhibitory Activity Assay

AuroraA (Upstate Discovery) or GSK3-β (Upstate Discovery) are diluted to 10 nM and 7.5 nM respectively in 25 mM MOPS, pH 7.00, 25 mg/ml BSA, 0.0025% Brij-35, 1.25% glycerol, 0.5 mM EDTA, 25 mM MgCl₂, 0.025% β-mercaptoethanol, 37.5 mM ATP and and 10 μl mixed with 10 μl of substrate mix. The substrate mix for Aurora is 500 μM Kemptide peptide (LRRASLG, Upstate Discovery) in 1 ml of water with 35 μCi γ³³P-ATP. The substrate mix for GSK3-β is 12.5 μM phospho-glycogen synthase peptide-2 (Upstate Discovery) in 1 ml of water with 35 μCi γ³³P-ATP. Enzyme and substrate are added to 96 well plates along with 5 μl of various dilutions of the test compound in DMSO (up to 2.5%). The reaction is allowed to proceed for 30 minutes (Aurora) or 3 hours (GSK3-β) before being stopped with an excess of ortho-phosphoric acid (5 μl at 2%). The filtration procedure is as for Activated CDK2/CyclinA assay above.

### Example 48

CDK Selectivity Assays

Compounds of the invention were tested for kinase inhibitory activity against a number of different kinases using the general protocol described in Example 47, but modified as set out below.

Kinases are diluted to a 10× working stock in 20 mM MOPS pH 7.0, 1 mM EDTA, 0.1% γ-mercaptoethanol, 0.01% Brij-35, 5% glycerol, 1 mg/ml BSA. One unit equals the incorporation of inmol of phosphate per minute into 0.1 mg/ml histone H1, or CDK7 substrate peptide at 30° C. with a final ATP concentration of 100 uM.

The substrate for all the CDK assays (except CDK7) is histone H1, diluted to 10× working stock in 20 mM MOPS pH 7.4 prior to use. The substrate for CDK7 is a specific peptide diluted to 10× working stock in deionised water.

Assay Procedure for CDK1/cyclinB CDK2/cyclinA, CDK2/cyclinE, CDK3/cyclinE, CDK5/p35, CDK6/cyclinD3:

In a final reaction volume of 251 μl, the enzyme (5-10 mU) is incubated with 8 mM MOPS pH 7.0, 0.2 mM EDTA, 0.1 mg/ml histone H1, 10 mM MgAcetate and [γ-³³P-ATP] (specific activity approx 500 cpm/pmol, concentration as required). The reaction is initiated by the addition of Mg²+[γ-³³P-ATP]. After incubation for 40 minutes at room temperature the reaction is stopped by the addition of 5 μl of a 3% phosphoric acid solution. 10 ml ofthe reaction is spotted onto a P30 filter mat and washed 3 times for 5 mlnutes in 75 mM phosphoric acid and once in methanol prior to drying and counting.

Assay Procedure for CDK7/cyclinH/MAT1

In a final reaction volume of 25 μl, the enzyme (5-10 mU) is incubated with 8 mM MOPS pH 7.0, 0.2 mM EDTA, 500 μM peptide, 10 mM MgAcetate and [γ-³³P-ATP] (specific activity approx 500 cpm/pmol, concentration as required). The reaction is initiated by the addition of Mg²+[γ-³³P-ATP]. After incubation for 40 minutes at room temperature the reaction is stopped by the addition of 5 μl of a 3% phosphoric acid solution. 10 ml of the reaction is spotted onto a P30 filtermat and washed 3 times for 5 mlnutes in 75 mM phosphoric acid and once in methanol prior to drying and counting.

### Example 49

Anti-Proliferative Activity

The anti-proliferative activities of compounds of the invention were determined by measuring the ability of the compounds to inhibition of cell growth in a number of cell lines, inhibition of cell growth was measured using the Alamar Blue assay (Nociari, M. M, Shalev, A., Benias, P., Russo, C. Journal of Immunological Methods 1998, 213, 157-167). The method is based on the ability of viable cells to reduce resazurin to its fluorescent product resorufin. For each proliferation assay cells were plated onto 96 well plates and allowed to recover for 16 hours prior to the addition of inhibitor compounds for a frther 72 hours. At the end of the incubation period 10% (v/v) Alamar Blue was added and incubated for a further 6 hours prior to determination of fluorescent product at 535 nM ex/590 nM em. In the case of the non-proliferating cell assay cells were maintained at confluence for 96 hour prior to the addition of inhibitor compounds for a further 72 hours. The number of viable cells was determined by Alamar Blue assay as before. All cell lines were obtained from ECACC (European Collection of cell Cultures).

By following the protocol set out above, compounds of the invention were found to inhibit cell growth in a number of cell lines.

### Example 50

Measurement of Inhibitory Activity Against Glycogen Synthase Kinase-3 (GSK-3)

GSK3β (human) is diluted to a 10× working stock in 50 mM Tris pH 7.5, 0.1 mM EGTA, 0.1 mM sodium vanadate, 0.1% β-mercaptoethanol, 1 mg/ml BSA. One unit equals the incorporation of 1nmol of phosphate per minute phospho-glycogen synthase peptide 2 per minute.

In a final reaction volume of 25 μl , GSK3β (5-10 mU) is incubated with 8 mM MOPS 7.0, 0.2 mM EDTA, 20 μM YRRAAVPPSPSLSRHSSPHQS(p)EDEEE (phospho GS2 peptide), 10 mM MgAcetate and [γ-³³P-ATP] (specific activity approx 500 cpm/pmol, concentration as required). The reaction is initiated by the addition of Mg²+[γ-³³P-ATP]. After incubation for 40 minutes at room temperature the reaction is stopped by the addition of 5 μl of a 3% phosphoric acid solution. 10 μl of the reaction is spotted onto a P30 filter mat and washed 3 times for 5 minutes in 50 mM phosphoric acid and once in methanol prior to drying and counting.

### Example 51

Determination of Antifungal Activity

The antifungal activity of the compounds of the formula (I) is determined using the following protocol.

The compounds are tested against a panel of fungi including Candida parpsilosis, Candida tropicalis, Candida albicans-ATCC 36082 and Cryptococcus neoformans. The test organisms are maintained on Sabourahd Dextrose Agar slants at 4° C. Singlet suspensions of each organism are prepared by growing the yeast overnight at 27° C. on a rotating drum in yeast-nitrogen base broth (YNB) with amino acids (Difco, Detroit, Mich.), pH 7.0 with 0.05 M morpholine propanesulphonic acid (MOPS). The suspension is then centrifuged and washed twice with 0.85% NaCl before sonicating the washed cell suspension for 4 seconds (Branson Sonifier, model 350, Danbury, Conn.). The singlet blastospores are counted in a haemocytometer and adjusted to the desired concentration in 0.85% NaCl.

The activity of the test compounds is determined using a modification of a broth microdilution technique. Test compounds are diluted in DMSO to a 1.0 mg/ml ratio then diluted to 64 μg/ml in YNB broth, pH 7.0 with MOPS (Fluconazole is used as the control) to provide a working solution of each compound. Using a 96-well plate, wells 1 and 3 through 12 are prepared with YNB broth, ten fold dilutions of the compound solution are made in wells 2 to 11 (concentration ranges are 64 to 0.125 μg/ml). Well 1 serves as a sterility control and blank for the spectrophotometric assays. Well 12 serves as a growth control. The microtitre plates are inoculated with 10 μl in each of well 2 to 11 (final inoculum size is 10⁴ organisms/ml). Inoculated plates are incubated for 48 hours at 35° C. The IC50 values are determined spectrophotometrically by measuring the absorbance at 420 nm (Automatic Microplate Reader, DuPont Instruments, Wilmington, Del.) after agitation of the plates for 2 minutes with a vortex-mixer (Vorte-Genie 2 Mixer, Scientific Industries; Inc., Bolemia, N.Y.). The IC50 endpoint is defined as the lowest drug concentration exhibiting approximately 50% (or more) reduction of the growth compared with the control well. With the turbidity assay this is defined as the lowest drug concentration at which turbidity in the well is <50% of the control (IC50). Minimal Cytolytic Concentrations (MCC) are determined by sub-culturing all wells from the 96-well plate onto a Sabourahd Dextrose Agar (SDA) plate, incubating for 1 to 2 days at 35° C. and then checking viability.

### Example 52

Protocol for the Biological Evaluation of Control of in vivo Whole Plant Fungal Infection

Compounds of the formula (I) are dissolved in acetone, with subsequent serial dilutions in acetone to obtain a range of desired concentrations. Final treatment volumes are obtained by adding 9 volumes of 0.05% aqueous Tween-20™ or 0.01% Triton X-100™, depending upon the pathogen.

The compositions are then used to test the activity of the compounds of the invention against tomato blight (Phytophthora infestans) using the following protocol. Tomatoes (cultivar Rutgers) are grown from seed in a soil-less peat-based potting mixture until the seedlings are 10-20 cm tall. The plants are then sprayed to run-off with the test compound at a rate of 100 ppm. After 24 hours the test plants are inoculated by spraying with an aqueous sporangia suspension of Phytophthora infestans, and kept in a dew chamber overnight. The plants are then transferred to the greenhouse until disease develops on the untreated control plants. Similar protocols are also used to test the activity of the compounds of the invention in combatting Brown Rust of Wheat (Puccinia), Powdery Mildew of Wheat (Ervsiphe vraminis), Wheat (cultivar Monon), Leaf Blotch of Wheat (Septoria tritici), and Glume Blotch of Wheat (Leptosphaeria nodorum).

### Pharmacueutical Formulations

### Example 53

(i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

(ii) Capsule Formulation

A capsule formulation is prepared by mixing 100 mg of a compound of the formula (I) with 100 mg lactose and filling the resulting mixture into standard opaque hard gelatin capsules.

Equivalents

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-63. (canceled)

64. A compound of the formula (I): 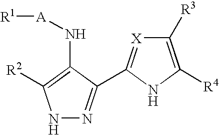
or a salt, N-oxide or solvate thereof;
wherein
X is CR⁵ or N;
A is a bond or —(CH₂)ₘ—(B)ₙ—;
B is C═O, NR^{g}(C═O) or O(C═O) wherein R^{g} is hydrogen or C₁₋₄ hydrocarbyl optionally substituted by hydroxy or C₁₋₄ alkoxy;
m is 0, 1 or 2;
n is O or 1;
R¹ is hydrogen, a carbocyclic or heterocyclic group having from 3 to 12 ring members, or an optionally substituted C₁₋₈ hydrocarbyl group;
R² is hydrogen, halogen, methoxy, or a C₁₋₄ hydrocarbyl group optionally substituted by halogen, hydroxyl or methoxy;
R³ and R⁴ are the same or different and each is selected from hydrogen, CN, C(O)R⁸, optionally substituted C₁₋₈ hydrocarbyl and carbocyclic or heterocyclic groups having from 3 to 12 ring members; and
R⁵ is hydrogen, a group R² or a group R¹⁰ wherein R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl;
X¹ is O, S or NR^{c} and X² is ═O, ═S or ═NR^{c}; and
R⁸ is selected from OR¹¹, SR¹¹ and NR¹²R¹³;
R¹¹ is selected from optionally substituted C₁₋₈ hydrocarbyl and carbocyclic or heterocyclic groups having from 3 to 12 ring members; and
one of R¹² and R¹³ is a group R¹¹ and the other of R¹² and R¹³ is hydrogen or C₁₋₄ alkyl; or R¹² and R¹³ and the nitrogen atom to which they are attached together form a saturated heterocyclic group having from 4 to 7 ring members and containing 1, 2 or 3 heteroatom ring members selected from N, O and S.

65. A compound according to claim 64 wherein X is N.

66. A compound according to claim 64 wherein m is 0 or 1, n is 1 and B is C═O.

67. A compound according to claim 64 wherein R² is hydrogen, fluorine or methyl, preferably hydrogen.

68. A compound according to claim 64 wherein R¹ is a monocyclic or bicyclic aryl or heteroaryl group of 3 to 12 ring members which is unsubstituted or substituted by one or more substituent groups R¹⁰ as defined in claim 64.

69. A compound according to claim 68 wherein the aryl or heteroaryl group R¹ is selected from phenyl, pyrazolo[1,5-a]pyridinyl, furanyl, indolyl, oxazolyl, thiazolyl, isoxazolyl, pyrrolyl, pyridyl, quinolinyl, 2,3-dihydro-benzo[1,4]dioxine, benzo[1,3]dioxole, 2,3-dihydrobenzofuranyl, imidazolyl and thienyl; each optionally substituted by one or more substituent groups R¹⁰ as defined in claim 64.

70. A compound according to claim 69 wherein the aryl or heteroaryl group R¹ is unsubstituted or is substituted by one or more substituent groups selected from the group R^{10a} consisting of halogen, hydroxy, trifluoromethyl, cyano, nitro, carboxy, heterocyclic groups having 5 or 6 ring members and up to 2 heteroatoms selected from O, N and S, a group R^{a}—R^{b} wherein R^{a} is a bond, O, CO, X³C(X⁴), C(X⁴)X³, X³C(X⁴)X³, S, SO, or SO₂, and R^{b} is selected from hydrogen, heterocyclic groups having 5 or 6 ring members and up to 2 heteroatoms selected from O, N and S, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having 5 or 6 ring members and up to 2 heteroatoms selected from O, N and S; wherein one or more carbon atoms of the C₁₋₈ hydrocarbyl group may optionally be replaced by O, S, SO, SO₂, X³C(X⁴), C(X⁴)X³ or X³C(X⁴)X³; X³ is O or S; and X⁴ is ═O or ═S.

71. A compound according to claim 64 wherein R¹ is a group as set out in Table 1:
TABLE 1

| | |
|---|---|
| | A1 |
| 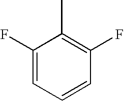 | |
| 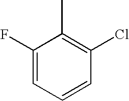 | A2 |
| 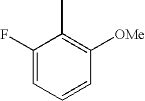 | A3 |
| 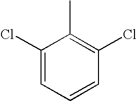 | A4 |
| 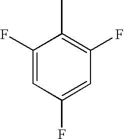 | A5 |
| 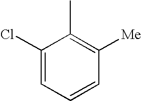 | A6 |
| 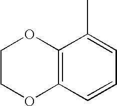 | A7 |
| 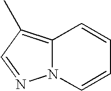 | A8 |
| 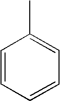 | A9 |
| | |
|---|---|
| | A10 |
| 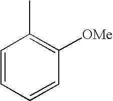 | |
| 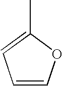 | A11 |
| 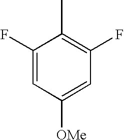 | A12 |
| 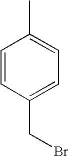 | A13 |
| 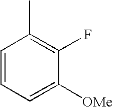 | A14 |
| 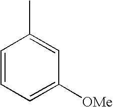 | A15 |
| 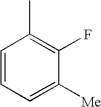 | A16 |
| 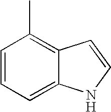 | A17 |
| 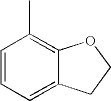 | A18 |
| 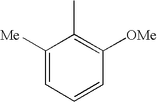 | A19 |
| 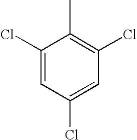 | A20 |
| 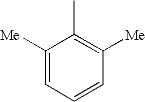 | A21 |
| 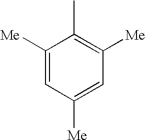 | A22 |
| 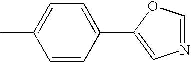 | A23 |
| 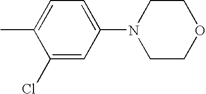 | A24 |
| 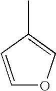 | A25 |
| 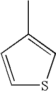 | A26 |
| 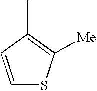 | A27 |
| 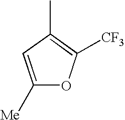 | A28 |
| 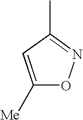 | A29 |
| 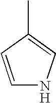 | A30 |
| 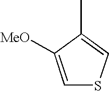 | A31 |
| 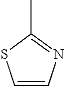 | A32 |
| 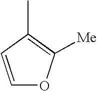 | A33 |
| 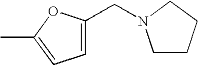 | A34 |
| 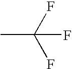 | A35 |
| 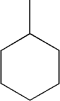 | A36 |
| 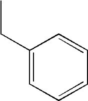 | A37 |
| 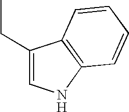 | A38 |
| 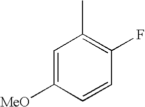 | A39 |
| 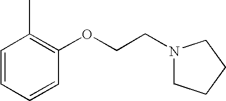 | A40 |
| 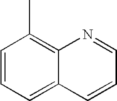 | A41 |
| 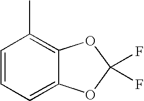 | A42 |
| 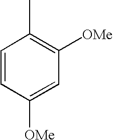 | A43 |
| 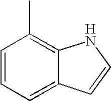 | A44 |
| 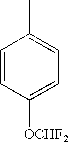 | A45 |
| 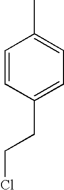 | A46 |
| 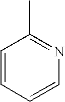 | A47 |
| 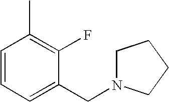 | A48 |
| 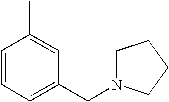 | A49 |
| 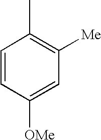 | A50 |
| 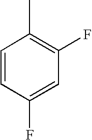 | A51 |
| 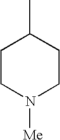 | A52 |
| 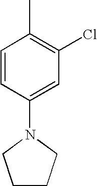 | A53 |
| 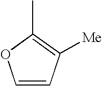 | A54 |
| 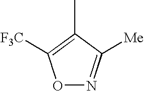 | A55 |
| 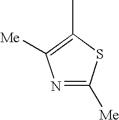 | A56 |
| 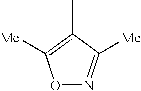 | A57 |
| 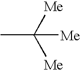 | A58 |
| 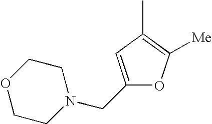 | A59 |
| 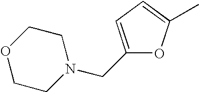 | A60 |
| 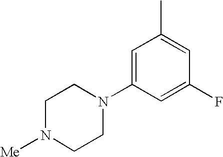 | A61 |
| 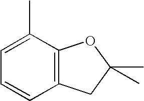 | A62 |
| 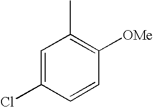 | A63 |

72. A compound according to claim 71 wherein R¹ is selected from groups A1, A3, A61, A62 and A63 in Table 1 of claim 71 (and more preferably A1).

73. A compound according to claim 64 wherein:
(A) one or both of R³ and R⁴ is or are other than hydrogen and is or are selected from optionally substituted C₁₋₈ hydrocarbyl and an optionally substituted carbocyclic or heterocyclic group selected from phenyl, naphthyl, thienyl, isoxazolyl, pyridyl, 2,3-dihydro-benzo[1,4]dioxine; or
(B) one of R³ and R⁴ is an optionally substituted group selected from phenyl, naphthyl, thienyl, isoxazolyl, pyridyl, 2,3-dihydro-benzo[1,4]dioxine, and the other one of R³ and R⁴ is an optionally substituted C₁₋₈ hydrocarbyl group;
wherein the optional substituents in (A) and (B) for the carbocyclic or heterocyclic groups are selected from the groups R¹⁰ and R^{10a} as defined in claim 64 or claim 70; and
wherein the optionally substituted C₁₋₈ hydrocarbyl group (A) and (B) is selected from:
(i) C₁₋₄ alkyl, hydroxy-C₁₋₄ alkyl and C₂₋₄ alkenyl; and
(ii) a C₁₋₈ hydrocarbyl group optionally substituted by a substituent selected from optionally substituted monocyclic carbocyclic and heterocyclic groups, NR¹²R¹³, C₁₋₄ alkoxy, halogen, hydroxy, C₁₋₄ alkylsulphonylamino, amino, mono- and di-C₁₋₄ alkylamino, wherein the alkyl residues of the C₁₋₄ alkoxy, mono- and di-C₁₋₄ alkylamino groups may themselves be further substituted by a substituent selected from NR¹²R¹³, C₁₋₄ alkoxy, hydroxy, C₁₋₄ alkylsulphonylamino, amino, and mono- and di-C₁₋₄ alkylamino, wherein R¹² and R¹³ are as defined in claim 64, and wherein the optional substituents for the carbocyclic and heterocyclic groups are selected from the group R¹⁰ as defined in any one of the preceding claims; or
(C) one of R³ and R⁴ is a group C(O)NR¹²R¹³ wherein R¹² and R¹³ and the nitrogen atom to which they are attached together form a saturated heterocyclic group having from 4 to 7 ring members and containing 1, 2 or 3 heteroatom ring members selected from N, O and S; or
(D) R³ and R⁴ are the same or different and are selected from C₁₋₄alkyl groups optionally substituted by halogen, hydroxy or methoxy.

74. A compound according to claim 64 wherein the imidazole group 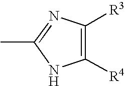
is selected from the groups B1 to B40 set out in Table 2:
TABLE 2
Examples of the Imidazole Group

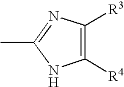

| | |
|---|---|
| | B1 |
| 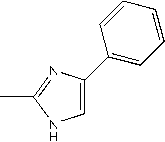 | |
| 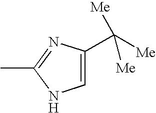 | B2 |
| 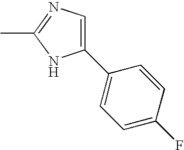 | B3 |
| 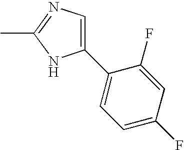 | B4 |
| 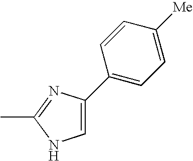 | B5 |
| 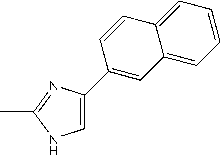 | B6 |
| 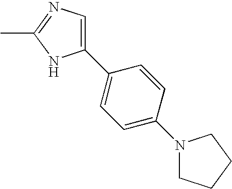 | B7 |
| 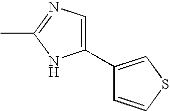 | B8 |
| 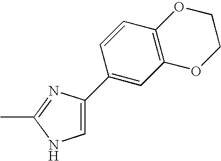 | B9 |
| | |
|---|---|
| | B10 |
| 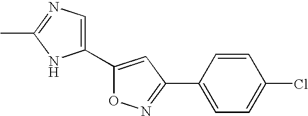 | |
| 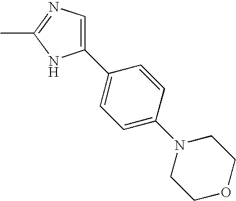 | B11 |
| 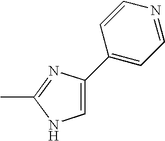 | B12 |
| 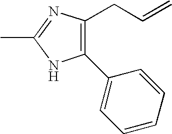 | B13 |
| 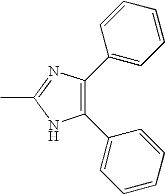 | B14 |
| 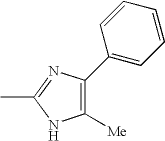 | B15 |
| 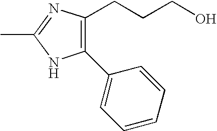 | B16 |
| 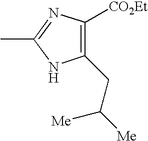 | B17 |
| 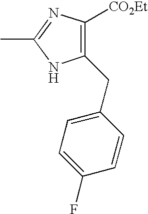 | B18 |
| 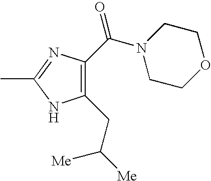 | B19 |
| 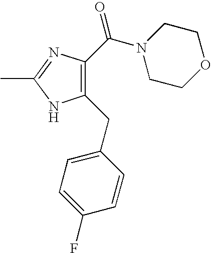 | B20 |
| 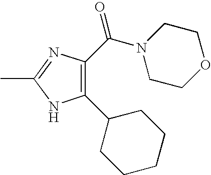 | B21 |
| 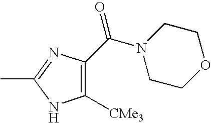 | B22 |
| 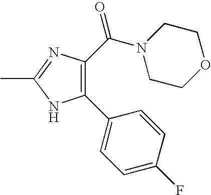 | B23 |
| 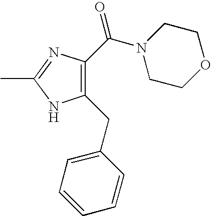 | B24 |
| 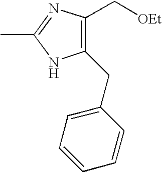 | B25 |
| 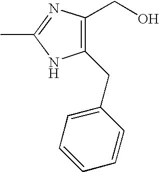 | B26 |
| 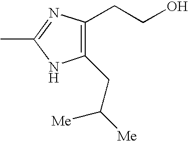 | B27 |
| 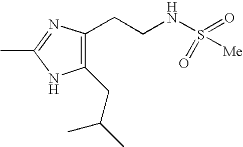 | B28 |
| 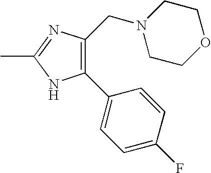 | B29 |
| 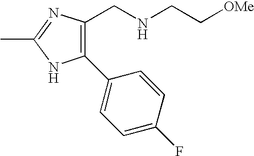 | B30 |
| 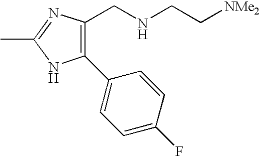 | B31 |
| 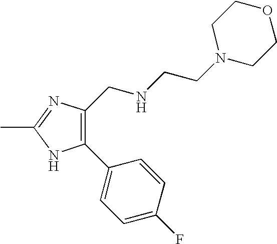 | B32 |
| 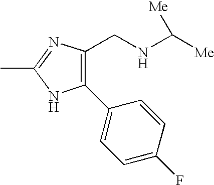 | B33 |
| 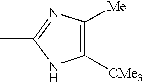 | B34 |
| 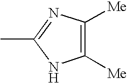 | B35 |
| 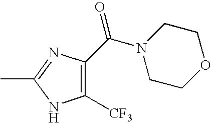 | B36 |
| 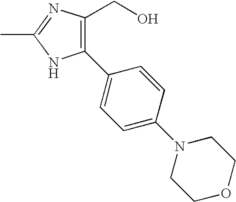 | B37 |
| 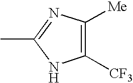 | B38 |
| 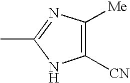 | B39 |
| 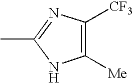 | B40 |
| 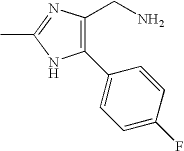 | B41 |

75. A compound according to claim 74 wherein the imidazole group is (i) selected from the groups B1 to B6, B8, B9 and B11 to B16 of Table 2, or is (ii) selected from B18, B19, B20, B22, B24, B25, B26, B27, B28, B29, B31, B34, B35, B37 and B38; or is (iii) selected from the groups B1 to B6, B8, B9, B11 to B13, B15 and B16; or is (iv) selected from the groups B2, B4, B12, B15 and B16.

76. A compound according to claim 64 in the form of a salt or solvate.

77. A pharmaceutical composition comprising a compound of the formula (I) as defined in claim 64 and a pharmaceutically acceptable carrier.

78. A method for the prophylaxis or treatment of a disease state or condition mediated by a cyclin dependent kinase, which method comprises administering to a subject in need thereof a compound of the formula (I) as defined in claim 64.

79. A method according to claim 78 wherein the disease state or condition is selected from proliferative disorders, viral infections, autoimmune diseases and neurodegenerative diseases.

80. A method according to claim 79 wherein the proliferative disorder is a cancer selected from breast cancer, ovarian cancer, colon cancer, prostate cancer, oesophageal cancer, squamous cancer, and non-small cell lung carcinomas.

81. A method of modulating a cellular process by inhibiting the activity of a cyclin dependent kinase using a compound of the formula (I) as defined in claim 64.

82. A method for treating a disease or condition comprising or arising from abnormal cell growth in a mammal, which method comprises administering to the mammal a compound of formula (I) as defined in claim 64 in an amount effective to inhibit abnormal cell growth.

83. A method for the prophylaxis or treatment of a disease state or condition mediated by glycogen synthase kinase-3, which method comprises administering to a subject in need thereof a compound of the formula (I) as defined in claim 64.


SEQUENCE LISTING (2 sequences)

SEQ ID NO: 1 - protein, 7 aa, Artificial Sequence
  Description of Artificial Sequence Synthetic Peptide
LRRASLG

SEQ ID NO: 2 - protein, 26 aa, Artificial Sequence
  Description of Artificial Sequence Synthetic Peptide
  MOD_RES (21): PHOSPHORYLATION
YRRAAVPPSPSLSRHSSPHQSEDEEE